(19) United States
(12) Patent Application Publication
(10) Pub. No.: US 2023/0292687 A1
(43) Pub. Date: Sep. 21, 2023

(54) Methods and Compositions for Improving Cold-Tolerance of Plants

(71) Applicant: Donald Danforth Plant Science Center, St. Louis, MO (US)

(72) Inventors: Todd C. Mockler, St. Louis, MO (US); Nadia Shakoor, St. Louis, MO (US); Scott Lee, St. Louis, MO (US); Erica Agnew, St. Louis, MO (US); Greg Ziegler, St. Louis, MO (US); Jennifer Arp, St. Louis, MO (US)

(21) Appl. No.: 17/917,746

(22) PCT Filed: Apr. 8, 2021
(86) PCT No.: PCT/US2021/026351

§ 371 Date: Oct. 7, 2022

Related U.S. Application Data
(60) Provisional application No. 63/006,935, filed on Apr. 8, 2020.

(51) Int. Cl. A01H 1/00; C07K 14/415; A01H 6/46; A01H 5/10

(52) U.S. Cl. CPC A01H 1/1225; C07K 14/415; A01H 6/4666; A01H 5/10

(57) ABSTRACT

Provided herein are a sorghum genomic region, alleles, and genotypes that are shown to be associated with a cold-stress tolerance phenotype. Also provided are genetically edited plastocyanin genes that result in a cold-stress tolerance phenotype. Also provided are cold-tolerant plants comprising an introgressed genomic region associated with cold-stress tolerance.

### CROSS REFERENCE TO RELATED APPLICATIONS

This application claims the benefit of U.S. Provisional Application 63/006,935, filed Apr. 8, 2020, which is incorporated by reference herein in its entirety.

### STATEMENT REGARDING FEDERALLY-SPONSORED RESEARCH AND DEVELOPMENT

This invention was made with government support under Cooperative Agreement Number DE-AR0000594 awarded by Advanced Research Projects Agency-Energy (ARPA-E), U.S. Department of Energy. The government has certain rights in the invention.

### REFERENCE TO SEQUENCE LISTING SUBMITTED ELECTRONICALLY

The content of the electronically submitted Sequence Listing in ASCII text file (Name: 47004_207976_SEQ LIST_ST25.txt; Size: 13,391 bytes; and Dated of Creation: Apr. 7, 2021) filed with the application is incorporated herein by reference in its entirety.

### BACKGROUND

Sorghum bicolor (L.) Moench, a C4 crop native to Africa and known for its drought and heat tolerance, is a promising bioenergy crop due to its ability to grow in marginal environments, and high potential for biomass yield (Brenton, Z. W., et al. (2016) Genetics, 204(1): 21-33). Early season planting, which extends the growing season of bioenergy sorghum and potentially increases exposure to rainfall is a strategy to maximize biomass yield (Yu, J., Tuinstra, M. R. (2001) Crop Science, 41(5): 1438-1443; Upadhyaya, H. D., et al. (2015) Genome, 59(2): 137-145). Moreover, cultivation in northern regions and at higher elevations could increase the available land for sorghum bioenergy production, without using land areas needed for food or feed crop production.

However, early planting and cultivation in temperate areas will not lead to higher biomass yields if plant development stalls. Due to its cold-sensitivity, planting sorghum in temperatures below 12-15° C. will diminish the yield (Burow. G., et al. (2011) Molecular Breeding, 28(3): 391-402; Salas Fernandez, M. G., Schoenbaum. G. R., Goggi, A. S. (2014) Crop Science, 54(6): 2631-2638; Chopra. R., et al. (2015) BMC Genomics, 16(1): 1040). Therefore, identification and development of sorghum accessions with enhanced tolerance to cold is vital to lengthen the growing season, expand growing regions, and to achieve higher total biomass yields.

Increased cold tolerance is only one of several traits necessary for improved biomass yield. Identification of ‘ideotype-positive’ and ‘ideotype-negative’ accessions can be used to select accessions with multiple positive characteristics that contribute to increased yield as well as other valuable traits for crop production. For example, accessions with high biomass, reduced height, and high water use efficiency (WUE) may be more desirable for breeding bioenergy sorghum. Alternatively, accessions that are tall but have low biomass and low WUE may be the least desirable for multiple reasons including a higher propensity for lodging (Esechie, H. A., Maranville, J. W., Ross, W. M. (1977) Crop Science, 17(4): 609-612). Crosses of these different ideotypes can, for example, also be used to create a nested association mapping (NAM) population to identify variation in traits and genes associated with multiple beneficial characteristics that contribute to the desired ideotype.

To identify accessions that can tolerate the lower temperatures of early spring and colder climates, the genetic basis of sorghum's response to cold stress and potential for cold adaptability must be better understood. Cold tolerance is a complex quantitative trait, and there is phenotypic variability for the degree of cold tolerance among sorghum accessions (Chopra, R., et al. (2017) BMC Plant Biology, 17(1): 12; Yu, J., et al. (2004) Field Crops Research, 85(1): 21-30). Natural genetic variation in sorghum's response to cold stress has also been identified (Ortiz, D., Hu, J., Salas Fernandez, M. G. (2017) Journal of Experimental Botany, 68(16): 4545-4557). Genome-wide association mapping of cold sensitivity traits in diverse germplasm is a promising approach to identify allelic variation that may be harnessed to improve the cold tolerance of sorghum.

### SUMMARY

Provided for herein is a method for obtaining a sorghum plant comprising in its genome at least one cold-stress tolerance genetic locus, the method comprising the steps of: a. genotyping a plurality of sorghum plants with respect to at least one genetic locus comprising a plastocyanin gene Sobic.007G033300 (SEQ ID NO: 1); and b. selecting a sorghum plant comprising in its genome at least one genetic locus comprising a genotype associated with cold-stress tolerance, based on said genotyping.

Also provided for herein is a method for producing a sorghum plant comprising in its genome at least one introgressed cold-stress tolerance genetic locus, the method comprising the steps of: a. crossing a first sorghum plant with a genotype associated with cold-stress tolerance in a genetic locus comprising a plastocyanin gene Sobic.007G033300 with a second sorghum plant comprising a genotype not associated with cold-stress tolerance in a genetic locus comprising a plastocyanin gene Sobic.007G033300 and at least one second polymorphic locus that is linked to the genetic locus comprising Sobic.007G033300 and that is not present in said first sorghum plant to obtain a population segregating for the cold-stress tolerance loci and said linked polymorphic locus; b. genotyping for the presence of at least two polymorphic nucleic acids in at least one sorghum plant from said population, wherein a first polymorphic nucleic acid is located in said genetic locus comprising Sobic.007G033300 and wherein a second polymorphic amino acid is a linked polymorphic locus not present in said first sorghum plant; and c. selecting a sorghum plant comprising a genotype associated with cold-stress tolerance and at least one linked marker found in said second sorghum plant comprising a non-cold-stress tolerance locus but not found in said first sorghum plant, thereby obtaining a sorghum plant comprising in its genome an introgressed cold-stress tolerance locus.

Also provided for herein is a method of identifying a sorghum plant that comprises a genotype associated with cold-stress tolerance, comprising: genotyping a sorghum plant for the presence of an allele in at least one genetic locus associated with cold-stress tolerance, wherein the genetic locus comprises a plastocyanin gene Sobic.007G033300, and denoting based on the genotyping that said sorghum plant comprises a genotype associated with cold-stress tolerance.

Also provided for herein is a sorghum plant made by the methods disclosed herein, wherein said sorghum plant comprises an introgressed cold-stress tolerance locus. Also provided for herein is a sorghum plant comprising an introgressed cold-stress tolerance genetic locus comprising a genotype associated with cold-stress tolerance in a genomic region comprising a plastocyanin gene Sobic.007G033300, wherein at least one marker linked to the introgressed cold-stress tolerance genetic locus found in said sorghum plant is characteristic of germplasm comprising a non-cold-stress tolerance genetic locus but is not associated with germplasm comprising the cold-stress tolerance genetic locus.

Also provided for herein is a method of producing a commercial crop seed lot of sorghum seeds comprising in their genomes at least one introgressed cold-stress tolerance genetic locus, the method comprising the steps of: a. producing a population of sorghum plants from the sorghum plant selected in step (c) of any one of claims 21-33 or the sorghum plant of any one of claims 46 to 56 comprising a genotype associated with cold-stress tolerance and at least one linked marker found in said second sorghum plant comprising a non-cold-stress tolerance locus but not found in said first sorghum plant; and b. harvesting a commercial seed lot, wherein the harvested crop seed lot comprises a plurality of seeds that comprise in their genomes at least one introgressed cold-stress tolerance locus.

Also provided for herein is a commercial crop seed lot of sorghum seeds made the methods disclosed herein. Also provided for herein is a commercial crop seed lot of sorghum seeds comprising a plurality of seeds that comprise in their genomes at least one introgressed cold-stress tolerance genetic locus comprising a genotype associated with cold-stress tolerance.

Also provided for herein is a method of growing a sorghum plant comprising in its genome at least one cold-stress tolerance genetic locus of any one of the preceding claims, the method comprising growing the plant under cold conditions sufficient to cause a deleterious effect in a non-cold-stress tolerant variety.

Provided for herein is an edited plastocyanin gene. In certain embodiments, the edited gene comprises (i) a variant polynucleotide encoding a plastocyanin protein variant or fragment thereof, wherein the variant polynucleotide comprises at least one nucleotide insertion, deletion, and/or substitution in comparison to the corresponding unedited wild-type polynucleotide sequence, and wherein the variant polynucleotide does not encode a wild-type plastocyanin protein. In certain embodiments, the variant polynucleotide is operably linked to a polynucleotide comprising a promoter. In certain embodiments, the edited gene comprises (ii) a variant polypeptide comprising a plastocyanin gene 3′ UTR, wherein the variant polynucleotide comprises at least one nucleotide insertion, deletion, and/or substitution in the 3′ UTR in comparison to the corresponding unedited wild-type polynucleotide sequence. In certain embodiments, the variant polynucleotide is operably linked to a polynucleotide comprising a promoter and/or a plastocyanin protein coding region. In certain embodiments, the edited gene comprises (iii) a variant polypeptide comprising a plastocyanin gene 5′ UTR, wherein the variant polynucleotide comprises at least one nucleotide insertion, deletion, and/or substitution in the 5′ UTR in comparison to the corresponding unedited wild-type polynucleotide sequence. In certain embodiments, the variant polynucleotide is operably linked to a polynucleotide comprising a promoter and/or a plastocyanin protein coding region. In certain embodiments, the edited gene comprises (iv) a variant polypeptide comprising a plastocyanin gene promoter, wherein the variant polynucleotide comprises at least one nucleotide insertion, deletion, and/or substitution in the promoter in comparison to the corresponding unedited wild-type polynucleotide sequence. In certain embodiments, the variant polynucleotide is operably linked to a polynucleotide comprising a plastocyanin protein coding region. In certain embodiments, the edited gene comprises (v) a variant polypeptide comprising a plastocyanin gene intron, wherein the variant polynucleotide comprises at least one nucleotide insertion, deletion, and/or substitution in the intron in comparison to the corresponding unedited wild-type polynucleotide sequence. In certain embodiments, the variant polynucleotide is operably linked to a polynucleotide comprising at least one plastocyanin gene exon. In certain embodiments, the edited gene comprises (vi) a variant polypeptide encoding a plastocyanin (a) transit peptide, vacuolar targeting peptide, and/or endoplasmic reticulum targeting peptide; (b) plastid targeting peptide; and/or (c) polyadenylation or transcriptional termination signal, wherein the variant polynucleotide comprises at least one nucleotide insertion, deletion, and/or substitution in (a), (b), and/or (c) in comparison to the corresponding unedited wild-type polynucleotide sequence. In certain embodiments, the polynucleotides of (a), (b), and/or (c) are operably linked to a polypeptide encoding a plastocyanin protein.

Certain embodiments provide for a plant nuclear or plastid genome comprising the edited plastocyanin gene of the disclosure, for example, as described above. Certain embodiments provide for a cell comprising the edited gene or nuclear or plastid genome. And, certain embodiments provide for a plant comprising the edited gene or nuclear or plastid genome and a plant part of the plant, wherein the plant part comprises the edited gene or nuclear or plastid genome.

Provided for herein is a method for obtaining a plant comprising the edited gene or plant nuclear or plastid genome of this disclosure, wherein the plant is cold-stress tolerant. In certain embodiments, the method comprises the steps of: (i) introducing the edited gene, the variant polynucleotide encoding the plastocyanin protein, the polynucleotide comprising the promoter, a fragment of said polynucleotides, or a combination of said polynucleotides into a plant cell, tissue, plant part, or whole plant; (ii) obtaining a plant cell, tissue, part, or whole plant wherein the edited gene, the variant polynucleotide encoding the plastocyanin protein, the polynucleotide comprising the promoter, a fragment of said polynucleotides, or a combination of said polynucleotides is integrated into the plant nuclear or plastid genome; and (iii) selecting a plant obtained from the plant cell, tissue, part or whole plant of step (ii) for expression of a variant plastocyanin protein, thereby obtaining a plant that is cold-stress tolerant.

Provide for herein a method for obtaining a plant comprising the edited gene or plant nuclear or plastid genome of this disclosure, wherein the plant is cold-stress tolerant. In certain embodiments, the method comprises introducing into a plant cell one or more gene editing molecules that target an endogenous plastocyanin gene to introduce at least one nucleotide insertion, deletion, and/or substitution into the endogenous plastocyanin gene. In certain embodiments, the method further comprising selecting a plant comprising a gene edited plastocyanin gene that expresses a variant plastocyanin protein; optionally, further comprising selecting a plant that is cold-stress tolerant.

Provided for herein is a method for obtaining a plant comprising the edited gene or nuclear or plastid genome of this disclosure, wherein the plant is cold-stress tolerant. In certain embodiments, the method comprises the steps of: (i) providing to a plant cell, tissue, part, or whole plant an endonuclease or an endonuclease and at least one guide RNA, wherein the endonuclease or guide RNA and endonuclease can form a complex that can introduce a double strand break at a target site in a nuclear or plastid genome of the plant cell, tissue, part, or whole plant; (ii) obtaining a plant cell, tissue, part, or whole plant wherein at least one nucleotide insertion, deletion, and/or substitution has been introduced into the corresponding wild-type polynucleotide sequence; and (iii) selecting a plant obtained from the plant cell, tissue, part or whole plant of step (ii) comprising the edited gene for expression of a plant plastocyanin protein variant or fragment, thereby obtaining a plant that is cold-stress tolerant.

Certain embodiments provide for a method of producing/breeding a cold-stress tolerant plant, the method comprising crossing a cold-stress tolerant plant of claim this disclosure with one or more other plants to produce a population of progeny plants and in certain embodiments further screening the population of progeny plants to identify cold-stress tolerant plants.

Also provided for herein a plant or a seed of said plant for use in a method of plant breeding, crop production, or for making a processed plant product.

### BRIEF DESCRIPTION OF THE DRAWINGS

FIG. 1A,B. FIG. 1A,B illustrates that traits of interest show distinct trends in heritability over time and temperature changes. (A) Heritability of traits over time at each temperature (15° C., 24° C., and 32° C.). (B) Density histograms of each trait analyzed show the day at which each corresponding trait is most heritable (height at 48 DAP, WUE at 42 DAP, hull area at 50 DAP, biomass at 50 DAP, and RGR at 24 DAP). Each trait was transformed to an approximately normal distribution using the Box-Cox method.

FIG. 2. FIG. 2 shows correlations among traits at 51 DAP. Correlations among biomass, height, hull area, RGR, and WUE at 51 DAP. Dark blue represents the strongest correlation, and dark orange represents the weakest correlation. The r² for each correlation is in the corresponding box below the colored circles.

FIG. 3A,B. FIG. 3A,B shows overlaps of high and low performing accessions at 51 DAP. Venn diagrams depict numbers of accessions represented in the lowest-performing accessions (A) or highest-performing accessions (B) that overlap for biomass, WUE, hull area, or height at 51 DAP.

FIG. 4A-D. FIG. 4A-D shows ideotype-positive and ideotype-negative accessions for WUE and biomass-associated traits. (A) Early and late ideotype-positive radar plots show accessions that are within both the top 10% of WUE and lowest 5% of height:biomass ratios. Each dashed square represents a percentile range: 0% is the center-most square, and 100% is the outermost square. Accessions whose phenotypes fall within a given percentile range for a given trait (height, height:biomass ratio, or WUE) reside on the inside of the corresponding square. (B) Early and late ideotype-negative radar plots show accessions that are within both the lowest 10% of WUE and the highest 5% of height:biomass ratios. (C-D) Heatmaps show the change in rank of early and late ideotype-positive and negative accessions over time and at each temperature stage for WUE (C) and height:biomass ratios (D). Dark blue indicates the lowest WUE (an ideotype-negative trait) and lowest height:biomass ratio (an ideotype-positive trait). Dark orange indicates the highest WUE (an ideotype-positive trait) and highest height:biomass ratio (an ideotype-negative trait).

FIG. 5A-C. FIG. 5A-C shows temporal profiles of WUE for the highest and lowest WUE accessions at each temperature phase. Each panel depicts the five highest and five lowest WUE accessions at each temperature phase. The colored lines show the changes in WUE for each of those accessions throughout the experiment as the plant developed and temperature changed. (A) WUE profiles for five highest and five lowest WUE accessions at 15° C. (B) WUE profiles for five highest and five lowest WUE accessions at 24° C. (C) WUE profiles for five highest and five lowest WUE accessions at 32° C.

FIG. 6A-C. FIG. 6A-C shows that BAP accessions separate into seven distinct temporal WUE clusters. (A) The temporal WUE profiles of BAP accessions from 12 DAP to 56 DAP separate into seven clusters. The growth curves represent the average WUE profile over time for each cluster. (B) Hierarchical dendrogram tree showing the segregation of the BAP accessions into 7 clusters, each represented with a different color. (C) Clustergram of the PCA-weighted mean of each cluster showing the divergence of the BAP accessions into the seven clusters, and the distances between the clusters. The cluster numbers and colors correspond to the dendrogram (B) and temporal WUE profiles (A).

FIG. 7. FIG. 7 shows a Manhattan plot of GWAS on biomass, growth, and WUE traits. The Manhattan plot depicts significant SNPs for each trait of interest and the corresponding −log₁₀(p) of the GWAS mixed linear model with no other SNPs included in the model.

FIG. 8. FIG. 8 shows that a temporal Manhattan plot of GWAS on biomass, growth, and WUE traits reveals transient QTL. The plot depicts highly significant SNPs corresponding to the traits of interest at specific DAP. The y-axis denotes the DAP, and the x-axis represents the sorghum chromosome. Black boxes highlight transient QTL that “turn on/off” with developmental stage and as the temperature increases.

FIG. 9. FIG. 9 shows a transient QTL associated with the hull area trait. The boxed region represents a transient hull area QTL at SNP 7:2,934,702 that “turns on/off” with developmental stage and as temperature increases. There are five genes located within 15 kb of the transient QTL. SNP 7:2,934,702 is directly on candidate gene Sobic.007G033300.

FIG. 10. FIG. 10 shows phenotypic differences in hull area associated with a SNP in a plastocyanin-like gene. The allele boxplot demonstrates the correlation of a “G” at SNP 7:2,934,702 with a significantly larger hull area at 27 DAP. Additional boxplots demonstrating this correlation for 28-30 DAP and 34-35 DAP can be found in S7.

FIG. 11. FIG. 11 is a map of the continental United States of America with, for purpose of this disclosure, the “northern portion” encircled.

FIG. 12A-D. FIG. 12A-D shows plasmid maps for four representative CRISPR-Cas9 gene editing constructs.

FIG. 13. FIG. 13 is an illustration of plastocyanin (Sevir.6G069300) Exon 1. The Mu1 variant of this disclosure is a 56 bp deletion between the two gRNA target sites; it results in a frameshift mutation. The Mu2 variant of this disclosure is a single base pair deletion of an “A”, which is within one of the gRNA target sites; it results in a nonsense mutation, generating a premature stop codon.

FIG. 14. FIG. 14 shows the sequence underlying the deletions in the Mu1 variant and the Mu2 variant.

FIG. 15. FIG. 15 shows the sequence variation for the 3′ UTR Mu1 variant of this disclosure in the 3′ UTR region of Sevir.6G069300, a single base insertion of “T”.

FIG. 16. FIG. 16 shows a heatmap of a kinship matrix showing correlation analysis among the 369 BAP accessions. The color histogram shows the distribution of coefficients of coancestry values in the whole kinship matrix. The color scale indicates the degree of correlation (white-yellow, low correlation; orange-red, strong correlation). C1: 25/29 accessions from Ethiopia, 26/29 photoperiod-sensitive. C2: 47/48 accessions from Ethiopia. C3: All photoperiod-insensitive accessions. C4: All cellulosic accessions; 29/30 photoperiod-sensitive.

FIG. 17A-C. FIG. 17A-C show Tables of Germination Data. (A) Accessions with an average germination time within 10 DAP. (B) Plants that did not germinate at any time. (C) Average germination times of accessions that germinated after 10 DAP, calculated from the three replicates for each BAP accession.

FIG. 18. FIG. 18 shows a table of heritability of traits.

FIG. 19. FIG. 19 shows a table ranking accessions for each trait at 51 DAP.

FIG. 20A-C. FIG. 20A-C show tables ranking accessions for each trait at different temperatures: (A) 15° C. Rankings; (B) 24° C. Rankings; and (C) 32° C. Rankings.

FIG. 21. FIG. 21 shows allele boxplots showing the correlation of a “G” at SNP 7:2,934,702 with a significantly larger hull area for 27-30 and 34-35 DAP.

FIG. 22A. FIG. 22A is a table showing all variants resulting in non-synonymous amino acid substitutions within the coding sequence of Sobic.007G033300 within BAP accessions and summarizes the potential impacts of variants based on SNPeff predictions. Variants with minor allele frequency <5% were removed. i Denotes the percentage of accessions within the BAP that carry the particular variant.

FIG. 22B. FIG. 22B is a boxplot showing hull area (27 DAP) for BAP accessions based on the presence of 1 to 8 moderate effect SNPs in gene Sobic.007G033300 resulting in non-synonymous amino acid substitutions (Alternate) versus accessions with no variants in comparison to reference (Reference). The analysis excluded accessions containing heterozygous alleles. Alternate accessions have significantly larger hull area (p=0.019) in comparison to reference accessions according to Welch's t-test.

FIG. 23A. FIG. 23A is a boxplot showing the relative growth rate for individuals within the BAP based on the presence of a missense allele resulting in a non-synonymous amino acid substitution (Chr 6:40312463, Asn184Lys). Accessions heterozygous at the SNP were not significantly different according to Welch's t-test.

FIG. 23B. FIG. 23B is a table showing all variants within the coding sequence of Sobic.006G057866 (PRR37/Ma1) in the BAP accessions and summarizes the potential impacts of variants based on SNPeff predictions. Variants with minor allele frequency <5% were removed. iDenotes the percentage of accessions within the BAP that carry the particular variant.

FIG. 24. FIG. 24 shows a scatterplot and regression demonstrating the correlation of area at 51 DAP to endpoint fresh weight.

FIG. 25A, B. FIG. 25A is an annotated illustration of the full length S. viridis plastocyanin gene (Sevir.6G069300) showing the target locations of the Exon 1 and 3′ UTR guide RNAs of the Exon 1 Mu1 56 bp deletion, the Exon 1 Mu2 “A” deletion, the Exon 1 putative Mu3 56 bp deletion, and the 3′ UTR Mu1 ‘T’ insertion. FIG. 25B is an annotated illustration of the full length Sorghum plastocyanin gene (Sobic.007G033300) showing the target locations of the 5′ UTR/Exon 1 and 3′ UTR guide RNAs.

FIG. 26. FIG. 26 shows sequences producing significant alignment to the sorghum plastocyanin gene Sobic.007G033300.1.

### DETAILED DESCRIPTION

### Overview

Sorghum bicolor is a promising cellulosic feedstock crop for bioenergy because of its potential for high biomass yields. However, in its early growth phases, sorghum is sensitive to cold stress, preventing early planting in temperate environments. Cold temperature adaptability is vital for the successful cultivation of both bioenergy and grain sorghum at higher latitudes and elevations, and for early season planting or to extend the growing season. Identification of genes and alleles that enhance biomass accumulation of sorghum grown under early cold stress would enable development of improved bioenergy sorghum through breeding or genetic engineering.

A genome-wide association study of bioenergy sorghum accessions phenotyped under early season cold stress revealed transient QTLs for highly heritable biomass and growth-related traits that appeared as the temperature increased and plants developed. Sorghum accessions clustered into multiple groups for each heritable trait with distinct growth profiles. GWAS identified candidate genes associated with growth traits and cold stress responses. The top-performing accessions with the highest growth-related trait values over time and temperature shifts may be useful for further genetic analysis and breeding or engineering efforts directed at biomass yield enhancements.

A high throughput imaging-based system was used to collect daily phenotypic measurements from a set of 369 diverse accessions from the sorghum Bioenergy Association Panel (BAP) genotyped with 232,303 high-quality single nucleotide polymorphism (SNP) markers (S1). Daily phenotypic measurements are more beneficial than single endpoint measurements because the response to cold stress can change over time and as plants develop. Therefore, daily imaging and phenotyping allowed for the comparison of the cold stress response in the BAP at different developmental stages.

High throughput image-based phenotyping was performed to track growth over time and under early cold stress in this sorghum panel. GWAS on the daily phenotypic data revealed genetic variation underlying the response to early-season cold stress and identified transient QTLs related to biomass, height, hull area, water use efficiency (WUE) and relative growth rate (RGR). Candidate genes associated with these phenotypes were identified for crop improvement through breeding or targeted genetic modifications.

The analysis identified a priori and novel candidate genes associated with growth-related traits and the temporal response to cold stress.

### Definitions

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “comprising” as used herein is to be construed as at least having the features to which it refers while not excluding any additional unspecified features. However, in embodiments provided herein where the term “comprising” is used, other embodiments where the phrases “consisting of” and/or “consisting essentially of” are substituted for the term “comprising” are also provided.

As used herein, the terms “include,” “includes,” and “including” are to be construed as at least having the features to which they refer while not excluding any additional unspecified features.

Where a term is provided in the singular, other embodiments described by the plural of that term are also provided. For example, the term “a” or “an” entity refers to one or more of that entity; “an allele,” is understood to represent “one or more alleles.” As such, the terms “a” (or “an”), “one or more,” and “at least one” can be used interchangeably herein.

Unless defined otherwise, technical and scientific terms used herein have the same meaning as commonly understood by one of ordinary skill in the art to which this disclosure is related.

Numeric ranges are inclusive of the numbers defining the range. Even when not explicitly identified by “and any range in between,” or the like, where a list of values is recited, e.g., 1, 2, 3, or 4, the disclosure specifically includes any range in between the values, e.g., 1 to 3, 1 to 4, 2 to 4, etc.

The headings provided herein are solely for ease of reference and are not limitations of the various aspects or aspects of this disclosure, which can be had by reference to the specification as a whole.

As used herein, an “allele” refers to one of two or more alternative forms of a genomic sequence at a given locus on a chromosome. When all the alleles present at a given locus on a chromosome are the same, that plant is homozygous at that locus. If the alleles present at a given locus on a chromosome differ, that plant is heterozygous at that locus.

As used herein, the term “denoting” when used in reference to a plant genotype refers to any method whereby a plant is indicated to have a certain genotype. Such indications of a certain genotype include, but are not limited to, any method where a plant is physically marked or tagged. Physical markings or tags that can be used include, but not limited to, a barcode, a radio-frequency identification (RFID) tag, a label, or the like. Indications of a certain genotype also include, but are not limited to, any entry into any type of written or electronic database whereby the plant's genotype is provided.

A “genetic locus” or “locus” is a position on a genomic sequence that is usually found by a point of reference; e.g., a short DNA sequence that is a gene, or part of a gene or intergenic region. A locus may refer to a nucleotide position at a reference point on a chromosome, such as a position from the end of the chromosome. While the genetic locus may be identified by a particular reference sequence, e.g., the Sorghum plastocyanin gene Sobic.007G033300 (SEQ ID NO: 1), it is understood that the locus can comprise various allelic forms or variants and still be considered the same locus or marker.

As used herein, “linkage” refers to relative frequency at which types of gametes are produced in a cross. For example, if locus A has genes “A” or “a” and locus B has genes “B” or “b” and a cross between parent I with AABB and parent B with aabb will produce four possible gametes where the genes are segregated into AB, Ab, aB and ab. The null expectation is that there will be independent equal segregation into each of the four possible genotypes, i.e. with no linkage ¼ of the gametes will of each genotype. Segregation of gametes into a genotypes differing from ¼ are attributed to linkage.

As used herein, the termed “linked”, when used in the context of markers and/or genomic regions, means that the markers and/or genomic regions are located on the same linkage group or chromosome.

As used herein, “polymorphism” means the presence of one or more variations of a nucleic acid sequence at one or more loci in a population of at least two members. The variation can comprise but is not limited to one or more nucleotide base substitutions, the insertion of one or more nucleotides, a nucleotide sequence inversion, and/or the deletion of one or more nucleotides.

As used herein, the term “single nucleotide polymorphism,” also referred to by the abbreviation “SNP,” means a polymorphism at a single site wherein the polymorphism constitutes any or all of a single base pair change, an insertion of one or more base pairs, and/or a deletion of one or more base pairs.

As used herein, “marker” means a detectable characteristic that can be used to discriminate between organisms. Examples of such characteristics include, but are not limited to, genetic markers, biochemical markers, morphological characteristics, and agronomic characteristics.

As used herein, “marker assay” means a method for detecting a polymorphism at a particular locus using a particular method. Marker assays thus include, but are not limited to, measurement of at least one phenotype (such as seed color, flower color, or other visually detectable trait as well as any biochemical trait), or genotyping such as by restriction fragment length polymorphism (RFLP), single base extension, electrophoresis, sequence alignment, allelic specific oligonucleotide hybridization (ASO), random amplified polymorphic DNA (RAPD), microarray-based polymorphism detection technologies, and the like.

As used herein, “genotype” means the genetic component of the phenotype and it can be indirectly characterized using markers or directly characterized by nucleic acid sequencing.

As used herein, “phenotype” means the detectable characteristics of a cell or organism which can be influenced by gene expression.

As used herein, the term “introgressed”, when used in reference to a genetic locus, refers to a genetic locus that has been introduced into a new genetic background. Introgression of a genetic locus can thus be achieved through both plant breeding methods or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion. In certain embodiments, introgression could thus be achieved by substitution of a cold-stress sensitive locus with a corresponding cold-stress tolerance locus or by conversion of a locus from a cold-stress sensitive to a cold-stress tolerance genotype.

As used herein, “quantitative trait locus (QTL)” means a locus that controls to some degree numerically representable traits that are usually continuously distributed.

As used herein, the term “event”, when used in the context of describing a transgenic plant, refers to a particular transformed plant line. In a typical transgenic breeding program, a transformation construct responsible for a trait is introduced into the genome via a transformation method. Numerous independent transformants (events) are usually generated for each construct. These events are evaluated to select those with superior performance.

As used herein, the term “sorghum” means Sorghum bicolor and includes all plant varieties that can be bred with sorghum, including wild sorghum species. In certain embodiments, sorghum plants from the species Sorghum bicolor and subspecies can be genotyped using the compositions and methods of the present disclosure.

As used herein, the term “bulk” refers to a method of managing a segregating population during inbreeding that involves growing the population in a bulk plot, harvesting the self-pollinated seed of plants in bulk, and using a sample of the bulk to plant the next generation.

As used herein, a polynucleotide is said to be “endogenous” to a given cell when it is found in a naturally occurring form and genomic location in the cell.

As used herein, the phrase “consensus sequence” refers to an amino acid, DNA or RNA sequence created by aligning two or more homologous sequences and deriving a new sequence having either the conserved or set of alternative amino acid, deoxyribonucleic acid, or ribonucleic acid residues of the homologous sequences at each position in the created sequence.

As used herein, the terms “edit,” “editing,” “edited” and the like refer to processes or products where insertions, deletions, and/or nucleotide substitutions are introduced into a genome. Such processes include methods of inducing homology directed repair and/or non-homologous end joining of one or more sites in the genome.

The phrases “genetically edited plant,” “edited plant,” and the like are used herein to refer to a plant comprising one or more nucleotide insertions, deletions, substitutions, or any combination thereof in the genomic DNA of the plant. Such genetically edited plants can be constructed by techniques including CRISPR/Cas endonuclease-mediated editing, meganuclease-mediated editing, engineered zinc finger endonuclease-mediated editing, and the like.

The term “heterologous,” as used herein in the context of a second polynucleotide that is operably linked to a first polynucleotide, refers to: (i) a second polynucleotide that is derived from a source distinct from the source of the first polynucleotide; (ii) a second polynucleotide derived the same source as the first polynucleotide, where the first, second, or both polynucleotide sequence(s) is/are modified from its/their original form; (iii) a second polynucleotide arranged in an order and/or orientation or in a genomic position or environment with respect to the first polynucleotide that is different than the order and/or orientation in or genomic position or environment of the first and second polynucleotides in a naturally occurring cell; or (iv) the second polynucleotide does not occur in a naturally occurring cell that contains the first polynucleotide. Heterologous polynucleotides include polynucleotides that promote transcription (e.g., promoters and enhancer elements), transcript abundance (e.g., introns, 5′ UTR, and 3′ UTR), translation, or a combination thereof as well as polynucleotides encoding peptides or proteins, spacer peptides, or localization peptides. In certain embodiments, a nuclear or plastid genome can comprise the first polynucleotide, where the second polynucleotide is heterologous to the nuclear or plastid genome. A “heterologous” polynucleotide that promotes transcription, transcript abundance, translation, or a combination thereof as well as polynucleotides encoding peptides, spacer peptides, or localization peptides can be autologous to the cell but, however, arranged in an order and/or orientation or in a genomic position or environment that is different than the order and/or orientation in or genomic position or environment in a naturally occurring cell. A polynucleotide that promotes transcription, transcript abundance, translation, or a combination thereof as well as polynucleotides encoding peptides, spacer peptides, or localization can be heterologous to another polynucleotide when the polynucleotides are not operably linked to one another in a naturally occurring cell. Heterologous peptides or proteins include peptides or proteins that are not found in a cell or organism as the cell or organism occurs in nature. As such, heterologous peptides or proteins include peptides or proteins that are localized in a subcellular location, extracellular location, or expressed in a tissue that is distinct from the subcellular location, extracellular location, or tissue where the peptide or protein is found in a cell or organism as it occurs in nature. Heterologous polynucleotides include polynucleotides that are not found in a cell or organism as the cell or organism occurs in nature.

The term “homolog” as used herein refers to a gene related to a second gene by identity of either the DNA sequences or the encoded protein sequences. Genes that are homologs can be genes separated by the event of speciation (see “ortholog”). Genes that are homologs can also be genes separated by the event of genetic duplication (see “paralog”). Homologs can be from the same or a different organism and can in certain embodiments perform the same biological function in either the same or a different organism.

The phrase “operably linked” as used herein refers to the joining of nucleic acid or amino acid sequences such that one sequence can provide a function to a linked sequence. In the context of a promoter, “operably linked” means that the promoter is connected to a sequence of interest such that the transcription of that sequence of interest is controlled and regulated by that promoter. When the sequence of interest encodes a protein that is to be expressed, “operably linked” means that the promoter is linked to the sequence in such a way that the resulting transcript will be efficiently translated. If the linkage of the promoter to the coding sequence is a transcriptional fusion that is to be expressed, the linkage is made so that the first translational initiation codon in the resulting transcript is the initiation codon of the coding sequence. Alternatively, if the linkage of the promoter to the coding sequence is a translational fusion and the encoded protein is to be expressed, the linkage is made so that the first translational initiation codon contained in the 5′ untranslated sequence associated with the promoter and the coding sequence is linked such that the resulting translation product is in frame with the translational open reading frame that encodes the protein. Nucleic acid sequences that can be operably linked include sequences that provide gene expression functions (e.g., gene expression elements such as promoters, 5′ untranslated regions, introns, protein coding regions, 3′ untranslated regions, polyadenylation sites, and/or transcriptional terminators), sequences that provide DNA transfer and/or integration functions (e.g., T-DNA border sequences, site specific recombinase recognition sites, integrase recognition sites), sequences that provide for selective functions (e.g., antibiotic resistance markers, biosynthetic genes), sequences that provide scoreable marker functions (e.g., reporter genes), sequences that facilitate in vitro or in vivo manipulations of the sequences (e.g., polylinker sequences, site specific recombination sequences) and sequences that provide replication functions (e.g., bacterial origins of replication, autonomous replication sequences, centromeric sequences). In the context of an amino acid sequence encoding a localization, spacer, linker, or other peptide, “operably linked” means that the peptide is connected to the polyprotein sequence(s) of interest such that it provides a function. Functions of a localization peptide include localization of a protein or peptide of interest to, e.g., an extracellular space or subcellular compartment. Functions of a spacer peptide include linkage of two peptides of interest such that the peptides will be expressed as a single protein.

As used herein, the term “polypeptide” is intended to encompass a singular “polypeptide” as well as plural “polypeptides,” and comprises any chain or chains of two or more amino acids. Thus, as used herein, a “peptide,” an “oligopeptide,” a “dipeptide,” a “tripeptide,” a “protein,” an “amino acid chain,” an “amino acid sequence,” “a peptide subunit,” or any other term used to refer to a chain or chains of two or more amino acids, are included in the definition of a “polypeptide,” (even though each of these terms can have a more specific meaning) and the term “polypeptide” can be used instead of, or interchangeably with any of these terms. The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids.

The phrases “percent identity” or “sequence identity” as used herein refer to the number of elements (i.e., amino acids or nucleotides) in a sequence that are identical within a defined length of two DNA, RNA or protein segments (e.g., across the entire length of a reference sequence) in an alignment resulting in the maximal number of identical elements, and is calculated by dividing the number of identical elements by the total number of elements in the defined length of the aligned segments and multiplying by 100.

The phrase “transgenic” refers to an organism or progeny thereof wherein the organism's or progeny organism's DNA of the nuclear or organellar genome contains an inserted exogenous DNA molecule of 10 or more nucleotides in length. The phrase “transgenic plant” refers to a plant or progeny thereof wherein the plant's or progeny plant's DNA of the nuclear or plastid genome contains an introduced exogenous DNA molecule of 10 or more nucleotides in length. Such introduced exogenous DNA molecules can be naturally occurring, non-naturally occurring (e.g., synthetic and/or chimeric), from a heterologous source, or from an autologous source.

As used herein, the phrase “cold-stress sensitive,” “cold-stress sensitivity,” “exhibits cold-stress sensitivity,” and the like, refers to undesirable phenotypic traits observed in certain plants, such as certain sorghum germplasms, after exposure to cold-stress Such undesirable phenotypic traits include, but are not limited to, leaf chlorosis, leaf necrosis, and plant death.

As used herein, the phrase “cold-stress tolerant,” “cold-stress tolerance,” “exhibits cold-stress tolerance,” and the like refers to either the absence or reduction of undesirable phenotypic traits observed after exposure to cold-stress in “cold-stress sensitive” sorghum germplasms.

As used herein, the term “alters” means to change a characteristic. For example, an altered protein activity could mean a decrease in activity, but it could also mean an increase or a change in the specificity of the activity.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Description

Sorghum is attractive as a bioenergy feedstock crop because it is heat and drought tolerant and can thrive in marginal environments. It is an ideal target for accelerated improvement through breeding and engineering because it has extensive genetic and phenotypic diversity but has not yet benefited from genomics or genetic modification like some other crops such as maize. Early season planting of sorghum provides the opportunity for an extended growing season with greater potential accumulation of biomass. However, as a tropical crop, sorghum is sensitive to cold stress. Identification of accessions that exhibit the highest WUE, RGR, height, hull area, and biomass under early cold stress conditions could facilitate genetic improvement of bioenergy sorghum for early-season planting and cultivation in colder temperatures.

Top performing BAP accessions for bioenergy-related traits under early cold stress were identified. The accessions with the highest and lowest rankings for each trait and multiple bioenergy-related traits were identified. Accession PI329299 was among the top accessions for hull area and height, PI452619 was a top accession for hull area and WUE, PI329403 was a top accession for hull area and WUE, and PI585461 was among the top accessions for both biomass and WUE. These top performing accessions, particularly the accessions that possess multiple advantageous traits, are promising candidates for sorghum bioenergy breeding programs and the development of additional genetic resources such as mapping populations (e.g., NAMs).

Ideotype-positive and ideotype-negative accessions as accessions that ranked high or low, respectively, for multiple traits or exhibited beneficial trait combinations were identified. The ideotype-positive phenotype is the combination of high WUE with a low height-to-biomass ratio, i.e., a plant that has high biomass but does not grow tall. This ideotype may be desirable for bioenergy sorghum because the required biomass accumulation may be attained with reduced water use and without the increased risk of lodging associated with taller plants. Five accessions were ideotype-positive in the early phase of the experiment when the temperature was 15° C. These accessions may be beneficial in breeding bioenergy sorghum that can be planted early in the season or grown in colder environments. Accession PI329517, which attained the ideotype-positive phenotype early under cold stress, and maintained it after the temperature increased, may be beneficial for early season planting, for long growth periods, or under conditions of reduced water availability. On the other hand, ideotype-negative accessions that have relatively low biomass accumulation and ranked low for WUE, but are tall may be undesirable as bioenergy sorghum because more water would be required for the plant to grow tall and thin, with a higher risk of lodging and decreased biomass accumulation. Even though these accessions may not be desirable for breeding purposes, identification of these extreme ideotypes may be useful for the development of structured populations for further genetic analysis.

The profiles of the traits over time and in response to temperature changes could be discerned, ranked, and clustered using the phenotypic data collected with single-day resolution. The BAP clustered into 6-8 distinct temporal profiles for each trait, identifying accessions that perform best under early cold stress, as well as at different temperatures and development stages. For example, accessions in WUE clusters two, six, and seven have the highest WUE at 15° C. Similarly, cluster six not only attains high WUE under 15° C. but is the cluster with the highest WUE attained under increased temperatures and therefore may perform well under drought conditions. Accession PI455221 was among the top-performing accessions for WUE at both 15° C. and 24° C., and PI329403 was among the top-performing accessions for WUE at 15° C. and 32° C., demonstrating that high WUE can be maintained as the plants develop and as the temperature increases.

GWAS revealed 2,305 highly significant SNPs associated with biomass, hull area, WUE, RGR, and height phenotypes. Significant SNPs that colocalized for multiple traits were identified, suggesting that these polymorphisms may be near tightly linked genes or genes with pleiotropic effects. By using the daily values for each phenotype in the GWAS, the temporal correlations of SNPs to traits were determined. With this approach, transient QTL that “turn on/off” at a specific developmental stage or with a change in temperature were able to be identified.

The highly significant SNPs identified by GWAS revealed 72 candidate genes with putative functions related to the bioenergy-relevant and cold-stress-responsive traits of interest. A transient QTL for RGR at 33 DAP mapped to the gene Sobic.006G057866, which encodes PRR37/Ma1 an a priori candidate gene that represses flowering in long days (Murphy. R. L., et al. (2011) Proceedings of the National Academy of Sciences, 108(39); 16469-16474). The observation that this transient QTL for RGR “turns on” at 33 DAP, a critical time for the transition to flowering in sorghum, and immediately after the temperature increased from 15° C., is consistent with a role for PRR37/Ma1 in growth and biomass accumulation through regulation of the transition from vegetative to reproductive development. Another transient QTL associated with hull area was significant from 27-30 DAP and again from 34-35 DAP. This transient QTL mapped to the gene Sobic.007G033300, which encodes a putative plastocyanin that may function in photosynthetic electron transfer. Additional sequence variation in this gene correlated with a significantly larger hull area from 27-30 DAP. These observations suggest that Sobic.007G033300 is important for biomass accumulation, consistent with known deleterious effects of cold on photosynthesis, in particular, the capacity for electron transport and photosystem protection (Liu, X., et al. (2018) Frontiers in Plant Science. 9: 1715). Studies have also implicated that plastocyanins affect yield in crops such as rice (Zhang, J. P., et al. (2017) Plant Physiology, 175: 1175-1185).

In accordance with the present disclosure, Applicants have discovered genomic regions, associated markers, and associated methods for identifying and associating genotypes that effect the cold-stress tolerance of plants. In certain embodiments, the plant is sorghum. For example, in one embodiment, a method of the disclosure comprises screening a plurality of germplasm entries displaying a heritable variation for at least one cold-stress tolerance trait wherein the heritable variation is linked to at least one genotype; and associating at least one genotype from the germplasm entries to at least one cold-stress tolerance trait. The methods of the present disclosure can be used with traditional breeding techniques as described below to more efficiently screen and identify genotypes affecting a cold-stress tolerance trait.

The use of markers to infer a phenotype of interest results in the economization of a breeding program by substituting costly, time-intensive phenotyping assays with genotyping assays. Further, breeding programs can be designed to explicitly drive the frequency of specific, favorable phenotypes by targeting particular genotypes (U.S. Pat. No. 6,399,855). Fidelity of these associations may be monitored continuously to ensure maintained predictive ability and, thus, informed breeding decisions (US Patent Application 2005/0015827). In this case, costly, time-intensive phenotyping assays required for determining if a plant or plants contains a genomic region associated with a “cold-stress tolerance” or “cold-stress sensitivity” phenotype can be supplanted by genotypic assays that provide for identification of a plant or plants that contain the desired genomic region that confers cold-stress tolerance.

Genomic Region, Locus, and Genotype Associated with a Cold-Stress Tolerance Phenotype

Provided in this disclosure is a genomic region that is shown herein to be associated with a cold-stress tolerance phenotype when present in certain allelic forms (cold-stress tolerant genetic locus of this disclosure). Also provided herein is a cold-stress tolerance genetic locus as well as genotypes, alleles, and allelic states associated with cold-stress tolerance. It is understood that for purposes of this disclosure, a variant plastocyanin gene of this disclosure is a genotype associated with cold-stress tolerance and a genetic locus comprising a variant plastocyanin gene of this disclosure is a cold-stress tolerance genetic locus.

A sorghum genomic region that is associated with a cold-stress tolerance phenotype when present in certain allelic forms comprises a genetic locus comprising a plastocyanin gene Sobic.007G033300 (SEQ ID NO: 1). In certain embodiments, the genetic locus is a genomic region between any of 1 Mb, 500 kb, 100 kb, 50 kb, or 10 kb telomere proximal and any of 1 Mb, 500 kb, 100 kb, 50 kb, or 10 kb centromere proximal of a plastocyanin gene Sobic.007G033300. In certain embodiments, the genetic locus consists of a plastocyanin gene Sobic.007G033300. A series of markers useful in practicing the methods of this disclosure are provided herewith in Table 1. In certain embodiments, the genotype associated with cold-stress tolerance comprises at least one polymorphic allele associated with cold-stress tolerance of at least one marker in the gene Sobic.007G033300 selected from the group consisting of Chr07:2934702, Chr07:2934028, Chr07:2934031, Chr07:2934040, Chr07:2934099, Chr07:2934128, Chr07:2934129, Chr07:2934187, and Chr07:2934291. In certain embodiments, the genotype associated with cold-stress tolerance comprises at least two, at least three, at least four, at least five, at least six, at least seven, at least eight, or at least nine of these polymorphic alleles. In certain embodiments, the genotype associated with cold-stress tolerance comprises at least one polymorphic allele associated with cold-stress tolerance of at least the marker Chr07.2934702 in the gene Sobic.007G033300. In certain embodiments, the genotype associated with cold-stress tolerance comprises at least one polymorphic allele associated with cold-stress tolerance of at least the marker Chr07.2934702 and also comprises at least two, at least three, at least four, at least five, at least six, at least seven, at least eight, or at least nine of the aforementioned polymorphic alleles. In certain embodiments, the genotype associated with cold-stress tolerance comprises an allelic state with at least one single nucleotide polymorphism (SNP) associated with cold-stress tolerance of at least one marker in Sobic.007G033300, wherein the SNP is selected from the group consisting of a G allele of SNP 7:2934702, a C allele of SNP 7:2934028, a G allele of SNP 7:2934031, a T allele of SNP 7:2934040, a C allele of SNP 7:2934099, an A allele of 7:2934128, a G allele of 7:2934129, a T allele of SNP 07:2934187, and a C allele replacing the nucleotides CCCG beginning at position 07:2934291. In certain embodiments, the genotype associated with cold-stress tolerance comprises at least two, at least three, at least four, at least five, at least six, at least seven, at least eight, or at least nine of said SNPs. In certain embodiments, the genotype associated with cold-stress tolerance comprises the allelic state of a G allele of SNP 7:2934702. In certain embodiments, the genotype associated with cold-stress tolerance comprises the allelic state of a G allele of SNP 7:2934702 and also comprises at least two, at least three, at least four, at least five, at least six, at least seven, at least eight, or at least nine of the aforementioned SNPs.

Without being bound by theory, certain alleles disclosed herein result in non-synonymous amino acid substitutions within the coding sequence of Sobic.007G033300. Certain alleles are associated with significantly improved (p≤0.019) growth and biomass accumulation following cold stress in comparison to the reference alleles.

TABLE 1
Markers spanning a genomic region associated with
a sorghum cold-stress tolerance phenotype.

| | | Allelic form |
|---|---|---|
| Marker or Locus Name | Reference Allele/ | Associated Cold- |
| (Chromosome Position) | Alternative Allele | Stress Tolerance |
| Chr07: 2934702 | /G | G |
| Chr07: 2934028 | T/C | C |
| Chr07: 2934031 | A/G | G |
| Chr07: 2934040 | A/T | T |
| Chr07: 2934099 | A/C | C |
| Chr07: 2934128 | C/A | A |
| Chr07: 2934129 | C/G | G |
| Chr07: 2934187 | C/T | T |
| Chr07: 2934291 | CCCG/C | C replacing |
| | | nucleotides CCCG |
| | | beginning at position |
| | | 07: 2934291 |

Additional genetic markers can be used either in conjunction with the markers provided in Table 1 or independently of the markers provided in Table 1 to practice the methods of this disclosure. Publicly available sorghum marker databases from which useful markers can be obtained are known to those of ordinary skill in the art. Given the provision herein of a genomic region associate with sorghum cold-stress tolerance, as well as an assortment of sorghum germplasms exhibiting either a “cold-stress sensitive” or “cold-stress tolerant” phenotype, additional markers located either within or near this genomic region that are associated with these phenotypes can be obtained by merely typing the new markers in the various known sorghum germplasms.

### Obtaining a Sorghum Plant Comprising in its Genome at Least One Cold-Stress Tolerance Genetic Locus.

Provided for herein is a method for obtaining a sorghum plant comprising in its genome at least one cold-stress tolerance genetic locus. In certain embodiments, the cold-stress tolerance genetic locus is a genetic locus comprising a plastocyanin gene Sobic.007G033300 and any of its embodiments as described in detail elsewhere herein. In certain embodiments, the method comprises the steps of (a) genotyping a plurality of sorghum plants with respect to at least one genetic locus comprising a plastocyanin gene Sobic.007G033300; and (b) selecting a sorghum plant comprising in its genome at least one genetic locus comprising a genotype associated with cold-stress tolerance, based on said genotyping. In certain embodiments, the genotype, polymorphic allele, allelic state, single nucleotide polymorphism, genetically edited variant, etc., associated with cold-stress tolerance is selected from any the embodiments as described in detail elsewhere herein. In certain embodiments, the selected sorghum plant exhibits cold-stress tolerance in comparison to a sorghum plant that is not considered cold-stress tolerant. In certain embodiments, a progeny of the selected sorghum plant exhibits cold-stress tolerance in comparison to a sorghum plant that is not considered cold-stress tolerant.

Once selected, a sorghum plant comprising in its genome a cold-stress tolerance genetic locus can used for breeding and/or in a breeding program to produce progeny comprising in their genomes the cold-stress tolerance locus. In certain embodiments, it is useful to use a sorghum plant comprising in its genome a cold-stress tolerance genetic locus to introduce the locus into germplasm that does not comprise the cold-stress tolerance genetic locus. Thus in certain embodiments, the selected sorghum plant having in its genome a cold-stress tolerance locus is crossed with a cold-stress sensitive sorghum plant to produce a progeny sorghum plant comprising in its genome at least one cold-stress tolerance genetic locus. In certain embodiments, the progeny plant exhibits cold-stress tolerance. Because sorghum is planted as a commercial crop, it is desirable to produce a population of sorghum plants comprising a plurality of sorghum plants comprising in their genomes the cold-stress tolerance genetic locus. Thus in certain embodiments, a sorghum plant having in its genome a cold-stress tolerance genetic locus is crossed with a cold-stress sensitive sorghum plant to produce a population of sorghum plants in comprising in their genomes at least one cold-stress tolerance genetic locus. The production of such a population of plants include the use of sorghum plant having in their genome an introgressed cold-stress tolerance genetic locus as described elsewhere herein and thus any of these embodiments apply to introgressed plants. In certain embodiments, the population of sorghum plants produced comprises a plurality of sorghum plants that exhibit cold-stress tolerance. It is understood that not all of the sorghum plants in a population, depending on how the population is produced and/or determined, will have inherited the cold-stress tolerance locus and/or will be cold-stress tolerant. In certain embodiments, at least 50%, at least 60%, at least 70%, at least 75%, at least 80%, at least 85%, at least 90%, at least 95%, at least 96%, at least 97%, at least 98%, at least 99%, or 100% of the sorghum plants in the population of sorghum plants produced exhibit cold-stress tolerance.

The starting sorghum plants to be used in the method for obtaining a sorghum plant comprising in its genome at least one cold-stress tolerance genetic locus can come from numerous sources disclosed elsewhere herein and/or known to those of ordinary skill in the art. In certain embodiments, the plurality of sorghum plants initially genotyped in the method comprises a population that is obtained by crossing a parent plant comprising at least one cold-stress tolerance locus with a parent plant comprising at least one non-cold-stress tolerance locus. In certain embodiments, the plurality of sorghum plants initially genotyped in the method comprises a population that is obtained by obtaining seed or progeny from a parental plant segregating for at least one cold-stress tolerance locus.

While the detection of a single allele having an allelic state associated with a cold-stress tolerance locus can be predictive of a cold-stress tolerance phenotype, in some cases it is useful to determine the allelic state of additional genetic markers, such as to strengthen the prediction. In any embodiments herein involving the genotyping of an allele, the embodiments also provide for genotyping for/determining, and optionally selecting/crossing/breeding a plant based thereon, the presence of a haplotype associated with cold-stress tolerance. Further, in some cases, it may be useful to determine whether a cold-stress tolerance allele and/or locus is present in a germplasm that naturally contains the allele and/or locus or whether the allele and/or locus has been artificially introduced (as to produce a new, non-naturally occurring plant), such as by introgression, into the germplasm. Thus in certain embodiments, the method further comprises genotyping for the presence of at least one additional marker. In some embodiments, the additional marker is associated with cold-tolerance. In some embodiments, the additional marker is linked to the genomic locus associated with cold-tolerance disclosed herein. In certain embodiments, the additional marker is not linked to the genomic locus associated with cold-tolerance.

As noted, not all sorghum plants comprising in their genomes at least one cold-stress tolerance genetic locus will exhibit cold-stress tolerance. Therefore in certain embodiments, the method further comprises exposing the selected sorghum plant or progeny thereof comprising the cold-stress tolerance locus to cold conditions sufficient to cause a deleterious effect in a non-cold-stress tolerant variety. In certain embodiments, this can be done in an experimental setting such as in a green house or other climate controlled environment. In certain embodiments, the plants can be planted in an open field wherein they are exposed to cold weather conditions, such as in the geographic regions described elsewhere herein.

Introgression of a Genomic Region Associated with a Cold-Stress Tolerance

Also provided herewith is unique germplasm, such as sorghum germplasms, comprising an introgressed genomic region that is associated with a cold-stress tolerance and methods of obtaining the same. Marker-assisted introgression involves the transfer of a chromosomal region, defined by one or more markers, from one germplasm to a second germplasm. Offspring of a cross that contain the introgressed genomic region can be identified by the combination of markers characteristic of the desired introgressed genomic region from a first germplasm (i.e., such as a cold-stress tolerance germplasm) and both linked and unlinked markers characteristic of the desired genetic background of a second germplasm (i.e., a cold-stress sensitive germplasm).

Certain embodiments of this disclosure provide for a method for producing a sorghum plant comprising in its genome at least one introgressed cold-stress tolerance genetic locus. In certain embodiments, the method comprises the steps of:
a. crossing a first sorghum plant with a genotype associated with cold-stress tolerance in a genetic locus of this disclosure with a second sorghum plant comprising a genotype not associated with cold-stress tolerance in said genetic locus and at least one second polymorphic locus that is linked to the genetic locus and that is not present in said first sorghum plant to obtain a population segregating for the cold-stress tolerance loci and said linked polymorphic locus;
b. genotyping for the presence of at least two polymorphic nucleic acids in at least one sorghum plant from said population, wherein a first polymorphic nucleic acid is located in said genetic locus and wherein a second polymorphic amino acid is a linked polymorphic locus not present in said first sorghum plant; and
c. selecting a sorghum plant based on the genotyping comprising a genotype associated with cold-stress tolerance as disclosed herein and at least one linked marker found in said second sorghum plant comprising a non-cold-stress tolerance locus but not found in said first sorghum plant, thereby obtaining a sorghum plant comprising in its genome an introgressed cold-stress tolerance genetic locus.

In certain embodiments, the second linked polymorphic locus is detected with a marker that is located within about 1000, 500, 100, 40, 20, 10, or 5 kilobases (Kb) of the cold-stress tolerance locus.

In certain embodiments, the method further comprises exposing the selected sorghum plant or progeny thereof comprising the introgressed cold-stress tolerance genetic locus to cold conditions sufficient to cause a deleterious effect in a non-cold-stress tolerant variety. In certain embodiments, the sorghum plant comprising in its genome the introgressed cold-stress tolerance locus exhibits cold-stress tolerance.

Identification of Plants Comprising a “Cold-Stress sensitivity” or “Cold-Stress Tolerance” Associated Genotype

Provided herein is a method of identifying a sorghum plant that comprises or does not comprise a genotype associated with cold-stress tolerance of this disclosure. In certain embodiments, the method comprises genotyping a sorghum plant for the presence of or absence an allele in at least one genetic locus associated with cold-stress tolerance as disclosed in detail elsewhere herein. In certain embodiments, the method comprises denoting based on the genotyping that said sorghum plant comprises or does not comprise a genotype associated with cold-stress tolerance. In certain embodiments, the method further comprises the step of selecting a denoted plant either comprising or not comprising a genotype associated with cold-stress tolerance from a population of plants.

In certain embodiments, the method comprises genotyping a sorghum plant for the presence of an allele in at least one genetic locus associated with cold-stress tolerance as disclosed elsewhere herein and denoting based on the genotyping that said sorghum plant comprises a genotype associated with cold-stress tolerance. In certain embodiments, the identified and/or selected sorghum plant comprises in its genome at least one introgressed cold-stress tolerance genetic locus exhibits cold-stress tolerance. Methods of identifying an introgressed locus are provided elsewhere herein.

Further, to observe the presence or absence of the cold-stress sensitive or cold-stress tolerant phenotypes, sorghum plants can be exposed to cold-stress such as conditions sufficient to cause a deleterious effect in a non-cold stress tolerant variety. In certain embodiments, an identified sorghum plant comprising a genotype associated with cold tolerance or a progeny thereof is cold-stress tolerant.

Sorghum Plants Comprising a Genomic Region Associated with Cold-Stress Tolerance

Provided herein are sorghum plants comprising a genomic region of this disclosure associated with cold-stress tolerance. In certain embodiments, the plant is a naturally occurring sorghum variety comprising genomic region associated with cold-stress tolerance, such as can be identified by the methods disclosed herein. In certain embodiments, the sorghum plant is made, such as by introgressing a cold-stress tolerance locus into a germplasm that comprises a cold-stress sensitive locus, or by genetic editing as disclosed elsewhere herein.

In certain embodiments, a sorghum plant comprises an introgressed cold-stress tolerance locus comprising a genotype associated with cold-stress tolerance as disclosed elsewhere herein, wherein at least one marker linked to the introgressed cold-stress tolerance locus found in said sorghum plant is characteristic of germplasm comprising a non-cold-stress tolerance locus but is not associated with germplasm comprising the cold-stress tolerance locus. In certain embodiments, the sorghum plant or a progeny thereof exhibits cold-stress tolerance.

### Additional Traits

It is contemplated that in any of the embodiments disclosed herein, a sorghum plant whether or not comprising in its genome a cold-stress tolerance genetic locus, can be non-transgenic and resistant to a herbicide. It is contemplated that in any of the embodiments disclosed herein, a sorghum plant whether or not comprising in its genome a cold-stress tolerance genetic locus, can contain a transgene that confers resistance to a herbicide. It is contemplated that in any of the embodiments disclosed herein, a sorghum plant whether or not comprising in its genome a cold-stress tolerance genetic locus, can contain a transgene that confers resistance to a herbicide and be resistant to an additional herbicide to which the plant does not contain a transgene conferring resistance thereto. In certain embodiments, the herbicide can be for example glyphosate, dicamba, imidazoline, metribuzin, 2,4-D, glufosinate, and/or bromoxynil. Thus, in certain embodiments, a method of this disclosure includes exposing a sorghum plant comprising in its genome a cold-stress tolerance genetic locus to an amount of herbicide sufficient to cause a deleterious effect in a sorghum plant that is not resistance to the herbicide.

For purposes of this disclosure, the presence and/or use of herbicide tolerance and herbicide application can apply to single plants or a population of plants such as used in a breeding program or grown from a commercial seed lot as disclosed elsewhere herein. For purposes of this disclosure herbicide tolerance, whether from a transgene or not, includes seeds that can be grown into herbicide tolerant plants.

### Molecular Assisted Breeding Techniques

Genetic markers that can be used in the practice of the instant disclosure include, but are not limited to, are Restriction Fragment Length Polymorphisms (RFLP), Amplified Fragment Length Polymorphisms (AFLP), Simple Sequence Repeats (SSR), Single Nucleotide Polymorphisms (SNP), Insertion/Deletion Polymorphisms (Indels), Variable Number Tandem Repeats (VNTR), and Random Amplified Polymorphic DNA (RAPD), and others known to those skilled in the art. Marker discovery and development in crops provides the initial framework for applications to marker-assisted breeding activities (US Patent Applications 2005/0204780, 2005/0216545, 2005/0218305, and 2006/00504538). The resulting “genetic map” is the representation of the relative position of characterized loci (DNA markers or any other locus for which alleles can be identified) along the chromosomes. The measure of distance on this map is relative to the frequency of crossover events between sister chromatids at meiosis.

As a set, polymorphic markers serve as a useful tool for fingerprinting plants to inform the degree of identity of lines or varieties (U.S. Pat. No. 6,207,367). These markers form the basis for determining associations with phenotype and can be used to drive genetic gain. The implementation of marker-assisted selection is dependent on the ability to detect underlying genetic differences between individuals.

Certain genetic markers for use in the present disclosure include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers.

In another embodiment, markers that include. but are not limited, to single sequence repeat markers (SSR), AFLP markers, RFLP markers, RAPD markers, phenotypic markers, isozyme markers, single nucleotide polymorphisms (SNPs), insertions or deletions (Indels), single feature polymorphisms (SFPs, for example, as described in Borevitz et al. 2003 Gen. Res. 13:513-523), microarray transcription profiles, DNA-derived sequences, and RNA-derived sequences that are genetically linked to or correlated with cold-stress tolerance loci, regions flanking cold-stress tolerance loci, regions linked to cold-stress tolerance loci, and/or regions that are unlinked to cold-stress tolerance loci can be used in certain embodiments of the instant disclosure.

In one embodiment, nucleic acid-based analyses for determining the presence or absence of the genetic polymorphism (i.e. for genotyping) can be used for the selection of seeds in a breeding population. A wide variety of genetic markers for the analysis of genetic polymorphisms are available and known to those of skill in the art. The analysis may be used to select for genes, portions of genes, QTL, alleles, or genomic regions (genotypes) that comprise or are linked to a genetic marker that is linked to or correlated with cold-stress tolerance loci, regions flanking cold-stress tolerance loci, regions linked to cold-stress tolerance loci, and/or regions that are unlinked to cold-stress tolerance loci can be used in certain embodiments of the instant disclosure.

Nucleic acid analysis methods (e.g., genotyping) provided herein include, but are not limited to, PCR-based detection methods (for example, TaqMan assays), microarray methods, mass spectrometry-based methods and/or nucleic acid sequencing methods. In one embodiment, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

A method of achieving such amplification employs the polymerase chain reaction (PCR) (Mullis et al. 1986 Cold Spring Harbor Symp. Quant. Biol. 51:263-273; European Patent 50,424; European Patent 84,796; European Patent 258,017; European Patent 237,362; European Patent 201,184; U.S. Pat. Nos. 4,683,202; 4,582,788; and 4,683,194), using primer pairs that are capable of hybridizing to the proximal sequences that define a polymorphism in its double-stranded form.

Methods for typing DNA based on mass spectrometry can also be used. Such methods are disclosed in U.S. Pat. Nos. 6,613,509 and 6,503,710, and references found therein.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981 and 7,250,252 all of which are incorporated herein by reference in their entireties. However, the compositions and methods of the present disclosure can be used in conjunction with any polymorphism typing method to type polymorphisms in genomic DNA samples. These genomic DNA samples used include but are not limited to genomic DNA isolated directly from a plant, cloned genomic DNA, or amplified genomic DNA.

For instance, polymorphisms in DNA sequences can be detected by hybridization to allele-specific oligonucleotide (ASO) probes as disclosed in U.S. Pat. Nos. 5,468,613 and 5,217,863. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening a plurality of polymorphisms. A single-feature polymorphism (SFP) is a polymorphism detected by a single probe in an oligonucleotide array, wherein a feature is a probe in the array. Typing of target sequences by microarray-based methods is disclosed in U.S. Pat. Nos. 6,799,122; 6,913,879; and 6,996,476.

Target nucleic acid sequence can also be detected by probe linking methods as disclosed in U.S. Pat. No. 5,616,464, employing at least one pair of probes having sequences homologous to adjacent portions of the target nucleic acid sequence and having side chains which non-covalently bind to form a stem upon base pairing of the probes to the target nucleic acid sequence. At least one of the side chains has a photoactivatable group which can form a covalent cross-link with the other side chain member of the stem.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283. SBE methods are based on extension of a nucleotide primer that is adjacent to a polymorphism to incorporate a detectable nucleotide residue upon extension of the primer. In certain embodiments, the SBE method uses three synthetic oligonucleotides. Two of the oligonucleotides serve as PCR primers and are complementary to sequence of the locus of genomic DNA which flanks a region containing the polymorphism to be assayed. Following amplification of the region of the genome containing the polymorphism, the PCR product is mixed with the third oligonucleotide (called an extension primer) which is designed to hybridize to the amplified DNA adjacent to the polymorphism in the presence of DNA polymerase and two differentially labeled dideoxynucleosidetriphosphates. If the polymorphism is present on the template, one of the labeled dideoxynucleosidetriphosphates can be added to the primer in a single base chain extension. The allele present is then inferred by determining which of the two differential labels was added to the extension primer. Homozygous samples will result in only one of the two labeled bases being incorporated and thus only one of the two labels will be detected. Heterozygous samples have both alleles present, and will thus direct incorporation of both labels (into different molecules of the extension primer) and thus both labels will be detected.

In another method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

In another embodiment, the locus or loci of interest can be directly sequenced using nucleic acid sequencing technologies. Methods for nucleic acid sequencing are known in the art and include technologies provided by 454 Life Sciences (Branford, CT), Agencourt Bioscience (Beverly, MA), Applied Biosystems (Foster City, CA), LI-COR Biosciences (Lincoln, NE), NimbleGen Systems (Madison, WI), Illumina (San Diego, CA), and VisiGen Biotechnologies (Houston, TX). Such nucleic acid sequencing technologies comprise formats such as parallel bead arrays, sequencing by ligation, capillary electrophoresis, electronic microchips, “biochips,” microarrays, parallel microchips, and single-molecule arrays, as reviewed by R. F. Service Science 2006 311:1544-1546.

The markers to be used in the methods of the present disclosure should preferably be diagnostic of origin in order for inferences to be made about subsequent populations. Experience to date suggests that SNP markers may be ideal for mapping because the likelihood that a particular SNP allele is derived from independent origins in the extant populations of a particular species is very low. As such, SNP markers appear to be useful for tracking and assisting introgression of QTLs, particularly in the case of genotypes.

### Gene Editing

Provided for herein are methods for producing a plant having a variant plastocyanin gene that confers cold-stress tolerance to plants. Thus, it is understood that for purposes of this disclosure, a variant plastocyanin gene of this disclosure is a genotype associated with cold-stress tolerance. In certain embodiments, the plant is a non-transgenic plant. These methods include, but are not limited to, gene editing tools such as CRISPR/Cas endonuclease-mediated editing, meganuclease-mediated editing, engineered zinc finger endonuclease-mediated editing, and traditional mutagenesis. For examples, certain embodiments comprise a precise gene editing in plant cells, callus, and/or germplasm explants (e.g. Sorghum) using CRISPR/Cas system mediated by homology direct repair (HDR). In certain embodiments, the modifications can confer cold-stress tolerance to plants which are regenerated and selected using an in vitro culture approach.

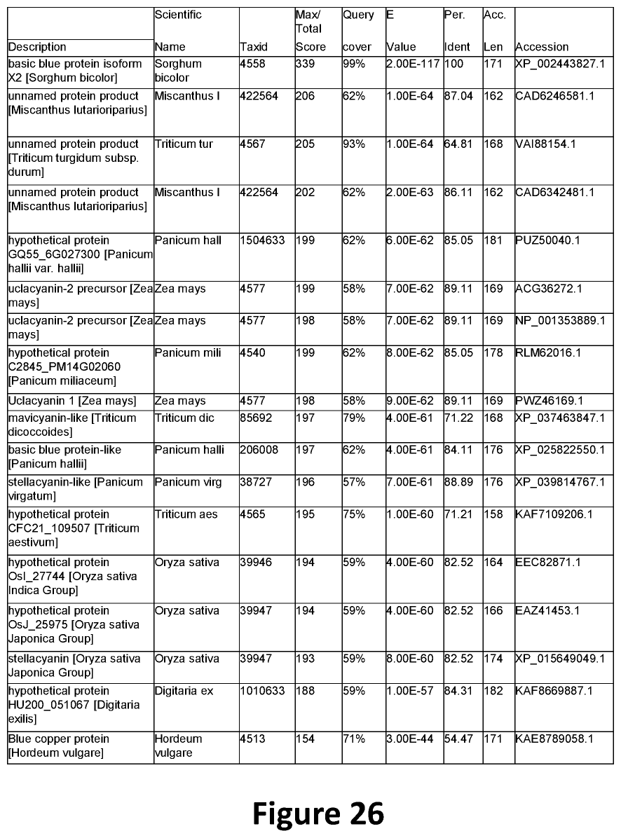

In certain embodiments, a genetically-edited plant comprising a plastocyanin variant gene and/or protein can be obtained by using techniques that provide for genome editing in the plant. In certain embodiments, a plant comprising an endogenous plastocyanin gene can be subjected to a genome editing technique wherein at least one nucleotide insertion, deletion, and/or substitution, in comparison to the corresponding unedited wild-type polynucleotide sequence is introduced, resulting in cold-stress tolerance. Examples of endogenous plant plastocyanin genes that can be edited to a cold-stress tolerant variant include, but are not limited to, the Sorghum gene Sobic.007G033300 (SEQ ID NO: 1) and the Seratia viridis gene Sevir.6G069300 (SEQ ID NO: 2). Other representative genes identified herein that can be edited to a cold-stress tolerant variant include those listed in FIG. 26, identified in a BLAST search that produced significant alignment to Sobic.007G033300. One of ordinary skill in the art would recognize that other potential target genes for gene editing according to this disclosure could similarly, or through other methods known in the field, be identified.

The at least one nucleotide insertion, deletion, and/or substitution can be made anywhere in the gene including, for example, in the promoter region, the exons, the introns, and the untranslated regions (5′ and 3′ UTRs). The insertion, deletion, and/or substitution can be minimal, for example, a one nucleotide insertion, deletion, or substitution, or it can be more extensive, e.g., a deletion or insertion of up to 2, 3, 4, 5, 10, 15, 20, 25, 50, or more nucleotides such as between any of about 1, 2, 3, 4, 5, 10, 15, 20, 25, or 50 and any of about 2, 3, 4, 5, 10, 15, 20, 25, 50 or 100 nucleotides, or multiple nucleotide substitutions (e.g., 2, 3, 4, 5, 6, 7, 8, 9, 10, 25, 50, or 100, or any integer inbetween). Examples of methods for plant genome editing with clustered regularly interspaced short palindromic repeats (CRISPR)-associated (Cas)-polynucleotide modification template technology and a Cas endonuclease are at least disclosed by Bortesi and Fisher et al., 2015; Svitashev et al., 2015; Kumar and Jain, 2015; and in US Patent Appl. Pub. Nos. 20150082478, 20150059010, 20190352655, and 2020157554, which are specifically incorporated herein by reference in their entireties. Examples of methods involving cytosine base editors and adenine base editors are at least disclosed by Kim, Nature Plants, 2018 March, 4(3):148-151; Komor et al. (2016) Nature, 533:420-424, Komor et al., Sci Adv. 2017 August; 3(8):eaao4774; and Gaudelli et al., (2017) Nature 551(7681):464-471 and in US Patent Appl. Pub. Nos. 20180362590 and 20180312828, which are specifically incorporated herein by reference in their entireties.

Gene editing molecules for inducing a genetic modification in the plant cell or plant protoplast of the systems, methods, and compositions provided herein include, but are not limited to: (i) a polynucleotide selected from the group consisting of an RNA guide for an RNA-guided nuclease, a DNA encoding an RNA guide for an RNA-guided nuclease; (ii) a nuclease selected from the group consisting of an RNA-guided nuclease, an RNA-guided DNA endonuclease, a type II Cas nuclease, a Cas9, a type V Cas nuclease, a Cpfl, a CasY, a CasX, a C2cl, a C2c3, an engineered nuclease, a codon-optimized nuclease, a zinc-finger nuclease (ZFN), a transcription activator-like effector nuclease (TAL-effector nuclease), Argonaute, a meganuclease or engineered meganuclease; (iii) a polynucleotide encoding one or more nucleases capable of effecting site-specific modification of a target nucleotide sequence; and/or (iv) a donor template polynucleotide. In certain embodiments, at least one delivery agent is selected from the group consisting of solvents, fluorocarbons, glycols or polyols, surfactants; primary, secondary, or tertiary amines and quaternary ammonium salts; organosilicone surfactants; lipids, lipoproteins, lipopolysaccharides; acids, bases, caustic agents; peptides, proteins, or enzymes; cell-penetrating peptides; RNase inhibitors; cationic branched or linear polymers; dendrimers; counter-ions, amines or polyamines, osmolytes, buffers, and salts; polynucleotides; transfection agents; antibiotics; chelating agents such as ammonium oxalate, EDTA, EGTA, or cyclohexane diamine tetraacetate, non-specific DNA double-strand-break-inducing agents; and antioxidants; particles or nanoparticles, magnetic particles or nanoparticles, abrasive or scarifying agents, needles or microneedles, matrices, and grids.

Thus, provided for herein is an edited plastocyanin gene. The edited plastocyanin gene can comprise an edit (e.g., at least one nucleotide insertion, deletion, and/or substitution) in any sequence/region of the gene, for example, in the promoter sequence, 5′ untranslated region (5′ UTR), exons, introns, 3′ UTR, etc., as long as the edit alters the expression and/or a characteristic of a gene product and/or activity of the plastocyanin protein. The at least one nucleotide insertion, deletion, and/or substitution can be short, e.g., one nucleotide, or it can be longer, e.g., comprising 10, 20, 30, 40, 50, 75, or more nucleotides that have been inserted, deleted, and/or substituted. In certain embodiments, the plastocyanin gene is disrupted or knocked-out. For example, a polynucleotide variant of a promoter or untranslated region could cause decrease in expression of the plastocyanin protein and thus an overall decrease in plastocyanin activity in a cell, even if the plastocyanin protein itself or its characteristics are unaltered. An edit in a protein coding region (e.g., exons) can result in a variant plastocyanin protein amino acid sequence. The polynucleotide variant can include a frameshift, missense, and/or nonsense mutation. Such polynucleotide variant can result in a protein variant with at least one amino acid insertion, deletion, substitution, and/or truncation of the protein. The protein variant can have a conservative or a non-conservative substitution. In certain embodiments, the edit alters the activity of the variant plastocyanin protein. In certain embodiments, the activity of plastocyanin protein is reduced or abolished in comparison to the the wild-type protein. In certain embodiments, when present in a plant and/or plant chromosome, the variant polynucleotide results in cold-stress tolerance.

In certain embodiments, the edited plastocyanin gene comprises (i) a variant polynucleotide encoding a plastocyanin protein variant or fragment thereof. The variant polynucleotide comprises at least one nucleotide insertion, deletion, and/or substitution in comparison to the corresponding unedited wild-type polynucleotide sequence and does not encode a wild-type plastocyanin protein. In certain embodiments, the variant polynucleotide is operably linked to a polynucleotide comprising a promoter for expression of the plastocyanin protein. In certain embodiments, a nucleotide insertion, deletion, and/or substitution is in Exon 1 or Exon 2 of the edited plastocyanin gene. For example, SEQ ID NO: 3 is the wild-type S. viridis plastocyanin Exon 1 sequence (similarly, SEQ ID NO: 5 is the wild-type Sorghum 5′ UTR/Exon 1 sequence). The polynucleotides of SEQ ID NO: 8 and SEQ ID NO: 10 represent edited variants of S. viridis Exon 1 and code for the plastocyanin protein/fragments of SEQ ID NO: 7 and SEQ ID NO: 9, respectively. In certain embodiments, the variant polynucleotide encodes a plastocyanin protein variant or fragment thereof having altered/reduced activity in comparison to the corresponding wild-type plastocyanin protein and/or the variant polynucleotide alters/decreases expression, transcription, intron splicing, and/or translation; alters the post-translational processing; and/or alters the sub-cellular localization of the plastocyanin gene, mRNA, and/or protein. In certain embodiments, when present in a plant and/or plant chromosome, the variant polynucleotide results in cold-stress tolerance.

In certain embodiments, the edited plastocyanin gene comprises (ii) a variant polypeptide comprising a plastocyanin gene 3′ UTR. The variant polynucleotide comprises at least one nucleotide insertion, deletion, and/or substitution in the 3′ UTR in comparison to the corresponding unedited wild-type polynucleotide sequence. In certain embodiments, the variant polynucleotide is operably linked to a polynucleotide comprising a promoter and/or a plastocyanin protein coding region. For example, SEQ ID NO: 4 is the wild-type S. viridis plastocyanin 3′ UTR sequence (simlarly, SEQ ID NO: 6 is the wild-type Sorghum 3′ UTR sequence). The polynucleotide of SEQ ID NO: 11 represents an edited variant of the S. viridis 3′ UTR. In certain embodiments, the variant polynucleotide alters/decreases expression, transcription, intron splicing, and/or translation; alters the post-translational processing; and/or the alters the sub-cellular localization of the plastocyanin gene, mRNA, and/or protein in comparison to an otherwise identical plastocyanin gene having a wild-type 3′ UTR. In certain embodiments, the insertion, deletion, and/or substitution in the 3′ UTR results in altered/reduced plastocyanin activity in comparison to an otherwise identical plastocyanin gene having a wild-type 3′ UTR. In certain embodiments, when present in a plant and/or plant chromosome, the variant polynucleotide results in cold-stress tolerance.

In certain embodiments, the edited plastocyanin gene comprises (iii) a variant polypeptide comprising a plastocyanin gene 5′ UTR. The variant polynucleotide comprises at least one nucleotide insertion, deletion, and/or substitution in the 5′ UTR in comparison to the corresponding unedited wild-type polynucleotide sequence. In certain embodiments, the variant polynucleotide is operably linked to a polynucleotide comprising a promoter and/or a plastocyanin protein coding region. For example, SEQ ID NO: 5 is the wild-type Sorghum 5′ UTR/Exon 1 sequence. In certain embodiments, the variant polynucleotide alters/decreases expression, transcription, intron splicing, and/or translation; alters the post-translational processing; and/or the alters the sub-cellular localization of the plastocyanin gene, mRNA, and/or protein in comparison to an otherwise identical plastocyanin gene having a wild-type 5′ UTR. In certain embodiments, the insertion, deletion, and/or substitution in the 5′ UTR results in altered/reduced plastocyanin activity in comparison to an otherwise identical plastocyanin gene having a wild-type 5′ UTR. In certain embodiments, when present in a plant and/or plant chromosome, the variant polynucleotide results in cold-stress tolerance.

In certain embodiments, the edited plastocyanin gene comprises (iv) a variant polypeptide comprising a plastocyanin gene promoter. The variant polynucleotide comprises at least one nucleotide insertion, deletion, and/or substitution in the promoter in comparison to the corresponding unedited wild-type polynucleotide sequence. In certain embodiments, the variant polynucleotide is operably linked to a polynucleotide comprising a plastocyanin protein coding region. In certain embodiments, the variant polynucleotide alters/decreases expression, transcription, intron splicing, and/or translation; alters the post-translational processing; and/or the alters the sub-cellular localization of the plastocyanin gene, mRNA, and/or protein in comparison to an otherwise identical plastocyanin gene having a wild-type promoter. In certain embodiments, the insertion, deletion, and/or substitution in the promoter results in altered/reduced plastocyanin activity in comparison to an otherwise identical plastocyanin gene having a wild-type promoter. In certain embodiments, when present in a plant and/or plant chromosome, the variant polynucleotide results in cold-stress tolerance.

In certain embodiments, the edited plastocyanin gene comprises (v) a variant polypeptide comprising a plastocyanin gene intron. The variant polynucleotide comprises at least one nucleotide insertion, deletion, and/or substitution in the intron in comparison to the corresponding unedited wild-type polynucleotide sequence. In certain embodiments, the variant polynucleotide is operably linked to a polynucleotide comprising at least one plastocyanin gene exon. In certain embodiments, the variant polynucleotide alters/decreases expression, transcription, intron splicing, and/or translation; alters the post-translational processing; and/or the alters the sub-cellular localization of the plastocyanin gene, mRNA, and/or protein in comparison to an otherwise identical plastocyanin gene having a wild-type intron. In certain embodiments, the insertion, deletion, and/or substitution in the intron results in altered/reduced plastocyanin activity in comparison to an otherwise identical plastocyanin gene having a wild-type intron. In certain embodiments, when present in a plant and/or plant chromosome, the variant polynucleotide results in cold-stress tolerance.

Similarly, in certain embodiments, and edited plastocyanin gene comprises (iv) a variant polypeptide encoding (a) a transit peptide, a vacuolar targeting peptide, and/or an endoplasmic reticulum targeting peptide; (b) a plastid targeting peptide; and/or (c) a polyadenylation or transcriptional termination signal. The variant polynucleotide comprises at least one nucleotide insertion, deletion, and/or substitution in (a), (b), and/or (c) in comparison to the corresponding unedited wild-type polynucleotide sequence. In certain embodiments, the polynucleotides of (a), (b), and/or (c) are operably linked to a polypeptide encoding a plastocyanin protein. In certain embodiments, the variant polynucleotide alters/decreases expression, transcription, intron splicing, and/or translation; alters the post-translational processing; and/or the alters the sub-cellular localization of the plastocyanin gene, mRNA, and/or protein in comparison to an otherwise identical plastocyanin gene having a wild-type intron. In certain embodiments, the insertion, deletion, and/or substitution in the intron results in altered/reduced plastocyanin activity in comparison to an otherwise identical plastocyanin gene having a wild-type intron. In certain embodiments, when present in a plant and/or plant chromosome, the variant polynucleotide results in cold-stress tolerance.

In certain of any embodiments of an edited plastocyanin gene of this disclosure, the variant polynucleotide is integrated into the nuclear or plastid genome of a cell. In certain embodiments, the nuclear or plastid genome is of a plant cell. Thus, the disclosure provides for a plant nuclear or plastid genome comprising an edited plastocyanin gene of this disclosure. In certain embodiments, the variant polynucleotide is heterologous to the nuclear or plastid genome. In certain embodiments, the variant polynucleotide is operably linked to an endogenous promoter of the nuclear or plastid genome, for example, a wild-type plastocyanin promoter. In certain embodiments, the edited gene or the nuclear or plastid genome further comprises a wild-type or variant polynucleotide encoding (a) a transit peptide, a vacuolar targeting peptide, and/or an endoplasmic reticulum targeting peptide; (b) a plastid targeting peptide; and/or (c) a polyadenylation or transcriptional termination signal, wherein the polynucleotides of (a), (b), and/or (c) are operably linked to the polypeptide encoding the plastocyanin protein.

In certain embodiments, the nuclear or plastid genome is a monocot crop plant or a dicot crop plant nuclear or plastid genome and/or the edited plastocyanin gene is from a monocot crop plant or a dicot crop plant. For example, in certain embodiments, a monocot crop plant is selected from the group consisting of a corn, barley, oat, pearl millet, rice, sorghum, sugarcane, turf grass, and wheat. In certain embodiments, the nuclear or plastid genome is a C3 plant or a C4 plant nuclear or plastid genome and/or the edited plastocyanin gene is from a C3 plant or a C4 plant. Representative examples of C3 plants include barley, oats, rice, and wheat, alfalfa (lucerne), cotton, Eucalyptus, sunflower, soybeans, sugar beets, potatoes, and tobacco. Representative examples of C4 plants include maize, sugarcane, and sorghum.

Also provided for herein is a cell comprising the edited gene or nuclear or plastid genome of this disclosure. In certain embodiments, the cell is a plant, yeast, mammalian, or bacterial cell. In certain embodiment, the plant is a plant cell, such as from a plant disclosed elsewhere herein. In certain embodiments, the cell is a plant cell that is non-regenerable.

Also provided for herein is a plant comprising the edited gene or nuclear or plastid genome of this disclosure. In certain embodiments, the plant is a monocot crop plant or a dicot crop plant. For example, in certain embodiments, a monocot crop plant is selected from the group consisting of a corn, barley, oat, pearl millet, rice, sorghum, sugarcane, turf grass, and wheat. In certain embodiments, the plant is a C3 plant or a C4 plant. Representative examples of C3 plants include barley, oats, rice, and wheat, alfalfa (lucerne), cotton, Eucalyptus, sunflower, soybeans, sugar beets, potatoes, and tobacco. Representative examples of C4 plants include maize, sugarcane, and sorghum. In certain embodiments, the edited gene or nuclear or plastid genome confers to the plant cold-stress tolerance in comparison to a control plant that lacks the edited gene or nuclear or plastid genome. Thus, in certain embodiments, the plant is cold-stress tolerant. Further, certain embodiments provide for a plant part of a plant above, wherein the plant part comprises the edited gene or nuclear or plastid genome. In certain embodiments, the plant part is a seed, stem, leaf, root, tuber, flower, or fruit.

Also provided for herein is a seed produced by a plant of this disclosure wherein said seed comprises a detectable amount of the variant polynucleotide encoding a plastocyanin protein variant or fragment. In certain embodiments, the seed comprises a transgene comprising a heterologous variant polynucleotide. In certain embodiments, the seed comprises an endogenous edited gene comprising the variant polynucleotide. In certain embodiments, the seed is coated with a composition comprising an insecticide and/or a fungicide. Also provided for herein is a plant propagation material comprising the coated seed.

Certain embodiments provide for a plant or seed of this disclosure for use in a method of plant breeding, crop production, or for making a processed plant product.

This disclosure provides for a method for obtaining a cold-stress tolerant plant comprising the edited gene or plant nuclear or plastid genome disclosed herein. The plant can be any plant as described herein. In certain embodiments, the plant is a monocot crop plant or a dicot crop plant. For example, in certain embodiments, a monocot crop plant is selected from the group consisting of a corn, barley, oat, pearl millet, rice, sorghum, sugarcane, turf grass, and wheat. In certain embodiments, the plant is a C3 plant or a C4 plant. Representative examples of C3 plants include barley, oats, rice, and wheat, alfalfa (lucerne), cotton, Eucalyptus, sunflower, soybeans, sugar beets, potatoes, and tobacco. Representative examples of C4 plants include maize, sugarcane, and sorghum. Such method comprises the steps of: (i) introducing the edited gene, the variant polynucleotide encoding the plastocyanin protein, the polynucleotide comprising the promoter, a fragment of said polynucleotides, or a combination of said polynucleotides into a plant cell, tissue, plant part, or whole plant; (ii) obtaining a plant cell, tissue, part, or whole plant wherein the edited gene, the variant polynucleotide encoding the plastocyanin protein, the polynucleotide comprising the promoter, a fragment of said polynucleotides, or a combination of said polynucleotides is integrated into the plant nuclear or plastid genome; and (iii) selecting a plant obtained from the plant cell, tissue, part or whole plant of step (ii) for expression of a variant plastocyanin protein, thereby obtaining a plant that is cold-stress tolerant.

This disclosure also provides for a method for obtaining a cold-stress tolerant plant comprising the edited gene or plant nuclear or plastid genome disclosed herein. The plant can be any plant as described herein. In certain embodiments, the plant is a monocot crop plant or a dicot crop plant. For example, in certain embodiments, a monocot crop plant is selected from the group consisting of a corn, barley, oat, pearl millet, rice, sorghum, sugarcane, turf grass, and wheat. In certain embodiments, the plant is a C3 plant or a C4 plant. Representative examples of C3 plants include barley, oats, rice, and wheat, alfalfa (lucerne), cotton, Eucalyptus, sunflower, soybeans, sugar beets, potatoes, and tobacco. Representative examples of C4 plants include maize, sugarcane, and sorghum. Such method comprises introducing into a plant cell one or more gene editing molecules, as described in greater detail elsewhere herein, that target an endogenous plastocyanin gene to introduce at least one nucleotide insertion, deletion, and/or substitution into the endogenous plastocyanin gene. In certain embodiments, the method further comprises selecting a plant comprising a gene edited plastocyanin gene that expresses a variant plastocyanin protein. Certain embodiments further comprise selecting a plant that is cold-stress tolerant. Thus, in certain embodiments, the gene editing transforms a cold-stress susceptible plant or plant line to a cold-stress tolerant plant or plant line.

This disclosure also provides for a method for obtaining a cold-stress tolerant plant comprising the edited gene or nuclear or plastid genome of this disclosure. The plant can be any plant as described herein. In certain embodiments, the plant is a monocot crop plant or a dicot crop plant. For example, in certain embodiments, a monocot crop plant is selected from the group consisting of a corn, barley, oat, pearl millet, rice, sorghum, sugarcane, turf grass, and wheat. In certain embodiments, the plant is a C3 plant or a C4 plant. Representative examples of C3 plants include barley, oats, rice, and wheat, alfalfa (lucerne), cotton, Eucalyptus, sunflower, soybeans, sugar beets, potatoes, and tobacco. Representative examples of C4 plants include maize, sugarcane, and sorghum. Such method comprises the steps of first (i) providing to a plant cell, tissue, part, or whole plant an endonuclease or an endonuclease and at least one guide RNA, wherein the endonuclease or guide RNA and endonuclease can form a complex that can introduce a double strand break at a target site in a nuclear or plastid genome of the plant cell, tissue, part, or whole plant. Certain embodiments further include providing a template polynucleotide comprising the polynucleotide encoding a plastocyanin protein or a fragment thereof. Then (ii) obtaining a plant cell, tissue, part, or whole plant wherein at least one nucleotide insertion, deletion, and/or substitution has been introduced into the corresponding wild-type polynucleotide sequence. And then (iii) selecting a plant obtained from the plant cell, tissue, part or whole plant of step (ii) comprising the edited gene for expression of a plant plastocyanin protein variant or fragment, thereby obtaining a plant that is cold-stress tolerant. In certain embodiments the endonuclease is a Cas endonuclease. In certain embodiments, the guide RNA is a clustered regularly interspaced short palindromic repeats (CRISPR)-associated (Cas)-guide RNA.

This disclosure also provides for a method of producing/breeding a cold-stress tolerant plant. The plant can be any plant as described herein. In certain embodiments, the plant is a monocot crop plant or a dicot crop plant. For example, in certain embodiments, a monocot crop plant is selected from the group consisting of a corn, barley, oat, pearl millet, rice, sorghum, sugarcane, turf grass, and wheat. In certain embodiments, the plant is a C3 plant or a C4 plant. Representative examples of C3 plants include barley, oats, rice, and wheat, alfalfa (lucerne), cotton, Eucalyptus, sunflower, soybeans, sugar beets, potatoes, and tobacco. Representative examples of C4 plants include maize, sugarcane, and sorghum. Such method comprises crossing a cold-stress tolerant plant of this disclosure with one or more other plants to produce a population of progeny plants. In certain embodiments, the one or more of the other plants comprises an endogenous edited gene. In certain embodiments, the method further comprises screening the population of progeny plants to identify cold-stress tolerant plants. In certain embodiments, the population of plants is screened by genotyping to detect a variant polynucleotide encoding for plastocyanin protein variant or fragment. In certain embodiments, the method further comprises selecting a progeny plant based on its genotype and/or phenotype.

### Transgenic Plants

Certain embodiments also provide for the transgenic expression of cold-stress tolerance variants in transgenic plants. Methods of producing transgenic constructs and transgenic plants are well-known in the art. Expression cassettes that provide for expression of polypeptides in monocotyledonous plants, dicotyledonous plants, or both can be constructed. Such expression cassette construction can be effected either in a plant expression vector or in the genome of a plant. Expression cassettes are DNA constructs wherein various promoter, coding, and polyadenylation sequences are operably linked. In general, expression cassettes typically comprise a promoter that is operably linked to a sequence of interest, which is operably linked to a polyadenylation or terminator region. In certain instances including, but not limited to, the expression of transgenes in monocot plants, it can also be useful to include an intron sequence. When an intron sequence is included it is typically placed in the 5′ untranslated leader region of the transgene. In certain instances, it can also be useful to incorporate specific 5′ untranslated sequences in a transgene to enhance transcript stability or to promote efficient translation of the transcript.

The DNA constructs that comprise the plant expression cassettes can either be constructed in the plant genome by using site specific insertion of heterologous DNA into the plant genome, by mutagenizing the plant genome, and/or by introducing the expression cassette into the plant genome with a vector or other DNA transfer method. Vectors contain sequences that provide for the replication of the vector and covalently linked sequences in a host cell. For example, bacterial vectors will contain origins of replication that permit replication of the vector in one or more bacterial hosts. Agrobacterium-mediated plant transformation vectors typically comprise sequences that permit replication in both E. coli and Agrobacterium as well as one or more “border” sequences positioned so as to permit integration of the expression cassette into the plant chromosome. Such Agrobacterium vectors can be adapted for use in either Agrobacterium tumefaciens or Agrobacterium rhizogenes. Selectable markers encoding genes that confer resistance to antibiotics are also typically included in the vectors to provide for their maintenance in bacterial hosts.

A transgenic plant containing or comprising an expression vector can be obtained by regenerating that transgenic plant from the plant, plant cell, protoplast, or plant tissue that received the expression vector.

### Method of Producing A Commercial Seed Lot

To generate enough seed for commercial distribution, the seed of commercial crops can be gathered from a plurality of plants and pooled together to create a seed lot. A commercial seed lot of a crop preferably contains a plurality of seeds that share similar or identical characteristics such as species, variety, genetic makeup, and/or similar germination rates. Provided for herein is a method of producing a commercial crop seed lot of sorghum seeds comprising in their genomes at least one cold-stress tolerance genetic locus and/or genotype associated with cold-stress tolerance of this disclosure. In certain embodiments, the genetic locus is an introgressed locus as described herein. In certain embodiments, the method comprises the steps of: (a) producing a population of sorghum plants, such as by but not limited to methods described elsewhere herein, comprising a genotype associated with cold-stress tolerance; and (b) harvesting a commercial seed lot, wherein the harvested crop seed lot comprises a plurality of seed that comprise in their genomes at least one cold-stress tolerance genetic locus. In certain embodiments, the method comprises the steps of: (a) producing a population of sorghum plants, such by but not limited to methods described elsewhere herein, comprising a genotype associated with cold-stress tolerance and at least one linked marker found in said second sorghum plant comprising a non-cold-stress tolerance genetic locus but not found in said first sorghum plant; and (b) harvesting a commercial seed lot, wherein the harvested crop seed lot comprises a plurality of seed that comprise in their genomes at least one cold-stress tolerance genetic locus. In certain embodiments, the method comprises the steps of: (a) producing a population of sorghum plants, such by but not limited to methods described elsewhere herein, comprising an introgressed genomic cold-stress tolerance genetic locus; and (b) harvesting a commercial seed lot, wherein the harvested crop seed lot comprises a plurality of seed that comprise in their genomes at least one introgressed cold-stress tolerance genetic locus. Certain embodiments may include a combination of any of the above.

In certain embodiments, the seed lot comprises at least 100 seeds, at least 500 seeds, at least 1,000 seeds, at least 5,000 seeds, at least 10,000 seeds, at least 25,000 seeds, at least 50,000 seeds, or at least 100,000 seeds. It is contemplated that a harvested commercial crop seed lot of the disclosure will not necessarily comprise seed that contains 100% of seeds that comprise in their genomes at least one cold-stress tolerance genetic locus. For example, some amount of contamination of other seeds into the harvested seed lot may occur. However, a high percentage of seeds comprising an cold-stress tolerance genetic locus is attainable. In certain embodiments, the plurality of seeds that comprise in their genomes at least one cold-stress tolerance genetic locus constitute at least 85%, at least 90%, at least 91%, at least 92%, at least 93%, at least 94%, at least 95%, at least 96%, at least 97%, at least 98%, or at least 99% of the seed of the harvested seed lot. In certain embodiments, the plurality of seeds that comprise in their genomes at least one introgressed cold-stress tolerance genetic locus constitute at least 85%, at least 90%, at least 91%, at least 92%, at least 93%, at least 94%, at least 95%, at least 96%, at least 97%, at least 98%, or at least 99% of the seed of the harvested seed lot.

### Packaging and Distribution

In certain embodiments, such as for a method for producing a commercial crop seed lot, the seed of the harvested commercial crop seed lot is packaged into one or more bags. Such packaging results in one or more packaged seed bags. In one embodiment, the packaged seeds, such as in packaged seed bags, are further distributed to growers for use in crop production.

One of skill in the art will recognize that packaged seed bags of a commercial crop seed lot destined for distribution to growers for use in crop production will preferably comprise a large number of seeds. For example, at least one hundred seeds, at least one thousand seeds, at least ten thousand seeds, at least one hundred thousand seeds, or at least one million seeds.

### Commercial Crop Seed Lot

Provided for herein is a commercial crop seed lot wherein a plurality of seeds comprise at least one cold-stress tolerance genetic locus and/or genotype associated with cold-stress tolerance of this disclosure. In certain embodiments a commercial crop seed lot comprises a plurality of seeds comprising at least one such cold-stress tolerance genetic locus that has been introgressed. Commercial crop seed lots of sorghum seeds can be made by the methods described in detail elsewhere herein.

In certain embodiments, the seed lot comprise at least 100 seeds, at least 500 seeds, at least 1,000 seeds, at least 5,000 seeds, at least 10,000 seeds, at least 25,000 seeds, at least 50,000 seeds, or at least 100,000 seeds. Further, in certain embodiments, wherein the plurality of seeds that comprise in their genomes at least one cold-stress tolerance genetic locus constitute at least 85%, at least 90%, at least 91%, at least 92%, at least 93%, at least 94%, at least 95%, at least 96%, at least 97%, at least 98%, or at least 99% of the seed of the seed lot. In certain embodiments, the cold-stress tolerance genetic locus is an introgressed locus.

For certain crop species, cross-pollination of certain distinct plant lines can result in hybrid offspring exhibiting a highly desirable heterosis or hybrid vigor which advantageously provides increased yields of the desired crop. In one embodiment, the crossing of parental plants produces a commercial crop seed lot that provides plants that yield at least 5% more than plants produced by selfing either the male parent plants or the female parent plants used to obtain the commercial crop seed lot when the crossed plants and the selfed plants are grown under the same field conditions. In a further embodiment, the crossing of parental plants produces a commercial crop seed lot that provides plants that yield at least 10% more than plants produced by selfing either the male parent plants or the female parent plants used to obtain the commercial crop seed lot when the crossed plants and the selfed plants are grown under the same field conditions. In yet a further embodiment, the crossing of parental plants produces a commercial crop seed lot that provides plants that yield at least 15% more than plants produced by selfing either the male parent plants or the female parent plants used to obtain the commercial crop seed lot when the crossed plants and the selfed plants are grown under the same field conditions.

One of skill in the art will recognize that certain standards may be set—as may be set for certification—for a commercial crop seed lot. These standards may vary according to the crop selected and different classes of standards may exist such as “breeder,” “foundation,” “registered,” and “certified.”

One of skill in the art will recognize that the preceding standards are illustrative and that standards may vary depending on geographical location and as set by different regulatory entities. The standards disclosed herein are consistent with standards that are practiced in the field of commercial seed certification. Such standards are useful guidelines, however, the present disclosure is not to be interpreted as limited only to such standards or crops, but also to encompass other standards and crops as are known to those skilled in the art.

### Methods of Growing Cold-Stress Tolerant Sorghum Plants

Provide for herein is a method of growing a sorghum plant comprising in its genome at least one cold-stress tolerance genetic locus and/or genotype associated with cold-stress tolerance of this disclosure, whether an introgressed locus or not, under cold-stress conditions. Also included are progeny plants and populations of plants. In certain embodiments, the method comprises growing a sorghum plant under cold conditions sufficient to cause a deleterious effect in a non-cold-stress tolerant variety. In certain embodiments, the sorghum plant is grown at an ambient temperature of less than 15° C., a temperature that will negatively impact both seedling emergence and vigor.

In certain embodiments, sorghum plants are grown in a temperature controlled environment, such as a greenhouse or other enclosure, in which the plants are exposed to cold-stress conditions. In certain embodiments, plants are grown in an open field such as for large scale breeding, seed production, and crop production. When grown in an open field, the sorghum plants will be subject to the prevailing weather conditions.

Due to the seasons, certain times of the year are cooler and less amenable if not prohibitive to planting sorghum. One advantage of the cold-stress tolerant sorghum plants of the present disclosure is to allow planting, for example earlier in the year, to improve yields. In certain embodiments, cold-stress tolerant plants of this disclosure, progeny, or population produced therefrom are planted and/or grown in the month of February, March, or April. In certain embodiments, cold-stress tolerant plants of this disclosure, progeny, or population produced therefrom are planted and/or grown in the month April.

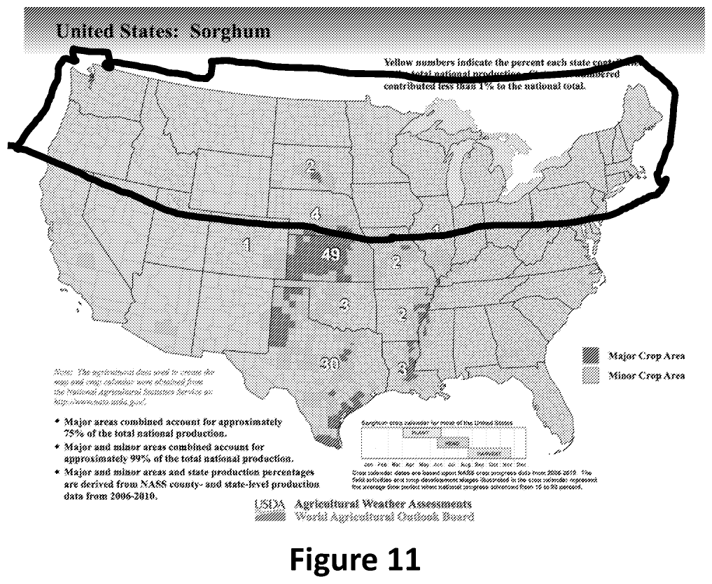

The climate and thus seasonal temperature varies with geographical location. In general in the norther hemisphere, the climate is cooler the farther north one goes. Thus, another advantage of the cold-stress tolerant sorghum plants of the present disclosure is to allow planting in geographical locations where sorghum hasn't been traditionally grown or is unable to grow because the climate is too cool. In certain embodiments, cold-stress tolerant plants of this disclosure, progeny, or population produced therefrom are planted and/or grown in the northern portion of the continental United States (FIG. 11). In certain embodiments, cold-stress tolerant plants of this disclosure, progeny, or population produced therefrom are planted and/or grown in the northern portion of the continental United States above about 35, 36, 37, 38, 39, 40, 41, 42, 43, 44, or 45 degrees north latitude. In certain embodiments, cold-stress tolerant plants of this disclosure, progeny, or population produced therefrom are planted and/or grown outside of the United States, such as in Europe, Japan, Korea, Russia, or China, above about 35, 36, 37, 38, 39, 40, 41, 42, 43, 44, or 45 degrees north latitude. In certain embodiments, cold-stress tolerant plants of this disclosure, progeny, or population produced therefrom are planted and/or grown in Alaska or Canada. In certain embodiments, cold-stress tolerant plants of this disclosure, progeny, or population produced therefrom are planted and/or grown in USDA Plant Hardiness Zones 5, 4, 3, 2, or 1. In certain embodiments, cold-stress tolerant plants of this disclosure, progeny, or population produced therefrom are planted and/or grown in outside of the United States, such as in Europe, Japan, Koran, Russia, or China, in a geographic location having a climate comparable to USDA Plant Hardiness Zones 5, 4, 3, 2, or 1.

Altitude can also affect temperature. In general, temperatures become cooler at higher altitudes. Thus, even in locations south of, for example the norther portion of the continental United States, sorghum may not be easily grown or grown at all at higher altitudes. Another advantage of the cold-stress tolerant sorghum plants of the present disclosure is to allow planting in geographical locations at altitudes where sorghum hasn't been traditionally grown or is unable to grow because the climate is too cool. In certain embodiments, cold-stress tolerant plants of this disclosure, progeny, or population produced therefrom are planted and/or grown at an altitude in geographical location wherein the ambient temperature drop below 15° C. during the growing season.

Further, it is within this disclosure to grow cold-stress tolerant plants of this disclosure, progeny, or population produced therefrom under a combination of any of the above temperature, time of year, geographical location, and/or altitude.

### EXAMPLES

### Example 1

### Materials and Methods

Plant Materials

This study used the sorghum Bioenergy Association Panel (BAP) (Brenton, Z. W., et al. (2016) Genetics. 204(1): 21-33). Details about the panel design, GBS genotyping, marker distribution, population structure, and linkage disequilibrium (LD) decay have been previously described (Brenton, Z. W., et al. (2016) Genetics. 204(1): 21-33). 369 BAP accessions were genotyped at 232,303 SNPs. The BAP accessions represent a racially, geographic, and phenotypically diverse selection of sorghum accessions, but is limited to accessions exhibiting key bioenergy traits, such as height, sensitivity to photoperiod, and delayed flowering.

Experimental Conditions

Three replicates of 369 BAP accessions (1131 plants) were planted in 600 g of Turface® in 8-inch tall tree pots, with +14−14−14 Osmocote (1.5 lb/cubic yard) fertilizer. The potted seeds were held overnight in a growth chamber at 32° C. (day)/22° C. (night). The next day, the pots were loaded into a carrier on an automated phenotyping system within a controlled-environment plant growth chamber. The phenotyping system moved the plants on a closed-loop conveyor path to stations for daily watering, weighing, and imaging. The plants were positioned in the growth chamber in a randomized block design and were rotated one-half lane each day to reduce edge effects. To study the effect of cold stress on these accessions, plants were grown at 15° C. (day)/15° C. (night) for 31 days, 24° C. (day)/19° C. (night) for 7 days, and 32° C. (day)/22° C. (night) for 18 days. The soil was maintained at 100% field water capacity by watering the plants twice daily to a target weight of 1192 grams. The target weight was calculated by adding the weight of the plant carrier (342 g), the water weight at saturation (250 g), and the weight of the Turface®-filled pot (600 g). Water was added after each carrier including the potted plant was weighed, and the volume of water added to reach the target weight of 1192 grams was recorded.

Image Collection and Phenotyping

Each plant passed through a visible light imaging chamber daily while on the automated phenotyping system. The imaging cameras recorded two side views and one top view image. As the plants grew, the fields of view on the cameras were adjusted so that the entire plant could be captured in each image. The optical zoom level was reduced for the top view and side view images at 19 DAP and 40 DAP. Scaling factors were calculated for both area and height using a reference object of known size, so that pixel areas across zoom levels were comparable. After eight weeks (56 DAP) the plants were removed from the phenotyping system. The shoot of each plant was cut at the base of the stem. Fresh weight measurements of the shoots were collected immediately.

The images were analyzed using Plant Computer Vision (PlantCV), an image-processing tool coded in Python (Fahlgren. N., et al. (2015) Molecular Plant, 8(10): 1520-1535). The pixel areas from the daily top view and two side view images of each plant were analyzed with PlantCV to generate measurements of area, hull area, height, RGR, and WUE.

The area was calculated by adding the pixel count from the top view image to the two side view images. Endpoint fresh weight measurements were correlated with area calculations at 51 DAP in order to estimate biomass (Fahlgren, N., et al. (2015) Molecular Plant, 8(10): 1520-1535). The values at 51 DAP were used for the correlation because, after that date, plants began to overlap and grow outside of the camera's field of view, and the resulting pixel counts were less indicative of the actual plant size.

The hull area is the convex hull calculated from the pixel count in the smallest area that includes a set of given points in a plane. WUE was calculated by dividing the derived area by the cumulative water added to each plant. Height was determined from the side view images.

The relative growth rate (RGR) was calculated as described in Hoffnann and Poorter, using estimator 2 to determine the RGR for each distinct time point (Hoffnann, W. A., & Poorter, H. (2002) Annals of Botany, 90(1): 37-42). Briefly, we calculated the natural log of all replicate area values in the experiment, then calculated the mean of the log-transformed values for two time points, t₁ and t₂. The mean value of the log-transformed areas for t₂, W₂, is subtracted from the mean value of the log-transformed areas for t₁, W₁, and then divided by the difference between t₂ and t₁ as shown:

$$RGR = \frac{\overline{ln ( {W}_{2} )} - \overline{ln ( {W}_{1} )}}{{t}_{2} - {t}_{1}}$$

Germination dates were determined for each plant based on the top view area measurements. The earliest DAP with a top view area measure was considered the germination date. The resulting germination date for the three replicates of each accession was averaged to determine the germination date for each accession.

Data Processing and Analysis

Image-derived phenotypic data were generated for 309 of the BAP accessions (S1). Phenotypic analysis was performed on the image-derived data using the R statistical software (R). Plants that never germinated or that died by 46 DAP were excluded from the analysis. A conservative initial outlier removal step was performed on each phenotype to remove likely artifacts from the image analysis. A data point was considered an outlier and excluded from further analysis if its value was greater than 40 median absolute deviations (MAD) from the median (Davies, L., Gather, U. (1993) Journal of the American Statistical Association, 88(423): 782-792). Raw measurements from PlantCV were smoothed using predicted values from a loess smoothing fit. These fitted values were used as the phenotypes for further analyses (Feldman, M. J., et al. (2017)PLoS Genetics, 13(6): e1006841).

The ‘lmer’ function in the lme4 R package was used to estimate variance components for broad-sense heritability (Bates, D., et al. (2015) Journal of Statistical Software., 67(1): 1-48). Heritability was calculated based on the subset of accessions that had three replicates that germinated. Broad-sense heritability was calculated as:

$${H}_{2} = \frac{{σ}_{genotype}^{2}}{{σ}_{total}^{2}}$$

After the heritability calculation, the median value of the replicates for each accession was used for further analysis. Traits were tested for normality and transformed as necessary using the Box-Cox procedure as implemented in R with the ‘boxcox’ function in the MASS package (Venables, W. N., Ripley, B. D. (2002) Modern applied statistics with S Statistics and Computing, pp. 271-300).

Hierarchical Clustering

Hierarchical clustering of each trait from 12-56 DAP was performed using the ‘hclust’ R function using a Euclidean distance matrix and the Ward agglomeration method (Murtagh, F., Legendre, P. (2014) Journal of Classification, 31(3): 274-295). Clustering results were visualized both as a dendrogram using the R package ‘dendextend’ and as a clustergram using the function ‘clustergranm.R’ (Galili T (2010) Clustergram: visualization and diagnostics for cluster analysis on the world wide web at “r-statistics.com/2010/06/clustergram-visualization-and-diagnostics-for-cluster-analysis-r-code”; Schonlau, M. (2002) The Stata Journal. 3: 316-327).

Ideotype Selection

Height-to-biomass ratios were calculated for each day and converted to a percentile with values between 0 and 100. The average percentile of the accessions for height-to-biomass ratio and WUE over the growing period, DAP 15 to 31 or DAP 39 to 56, for the early and late periods, respectively, was used to select ideotype accessions. Ideotype-positive accessions were defined as those with a height-to-biomass ratio in the bottom 5 percent of the population and a WUE in the top 10 percent of the population. Ideotype-negative accessions have a height-to-biomass ratio in the top 5 percent of the population and a WUE in the bottom 10 percent. Radar plots of the ideotype accessions were generated with the R package ‘fmsb’ (Nakazawa, M., & Nakazawa, M. M. (2018) fmsb: Functions for Medical Statistics Book with some Demographic Data. R package version 0.6.3). Heatmaps were generated using the R package ‘pheatmap’ (Kolde, R., & Kolde, M. R. (2018) pheatmap: Pretty Heatmaps. R package version 1.0.10).

Genome-Wide Association Study

Genotyping-by-sequencing (GBS) SNP markers for the BAP have been previously described (Brenton, Z. W., et al. (2016) Genetics. 204(1): 21-33). GWAS was performed using a multi-locus mixed linear model (MLMM) in R to identify loci associated with each trait of interest. The first three principal components of the genotype matrix were included as covariates in the mixed model in order to control for population structure (Price, A. L., et al. (2006) Nature Genetics, 38(8): 904). A kinship matrix, calculated from the genotype matrix using the Astle-Balding method in the ‘synbreed’ package, was also included as a random effect to control for familial and cryptic relatedness between accessions (Wimmer, V., Albrecht, T., Auinger, H. J., Schon, C. C. (2012) Bioinformatics, 28(15): 2086-2087; Astle, W., Balding, D. J. (2009) Statistical Science, 24(4): 451-471) (S2).

MLMM tests for association with the phenotype using a stepwise mixed model regression. In each step, the SNP with the most significant association to the phenotype is added to the model as a covariate. Stepwise addition of SNPs continues until the heritable variance estimate (pseudo-heritability) reaches 0 (Segura, V., et al (2012) Nature Genetics, 44(7): 825). A final set of high-confidence SNPs were selected as those that were included as covariates in either of the two optimal models selected by the MLMM software: the multiple-Bonferonni model or the extended BIC model (Segura, V., et al. (2012) Nature Genetics, 44(7): 825). In the multiple-Bonferroni model, all cofactors with a p-value below a Bonferroni corrected threshold are selected. Multiple-Bonferroni was the more stringent of the two models (Segura, V., et al. (2012) Nature Genetics, 44(7): 825). The extended BIC model selects a model based upon BIC penalized by the model complexity (Brenton, Z. W., et al. (2016) Genetics. 204(1): 21-33; Chen, J., Chen, Z. (2008) Biometrika, 95(3): 759-771).

The temporal GWAS results were analyzed using ZBrowse, an interactive browser that runs using R (Ziegler, G. R., Hartsock, R. H., & Baxter, I. (2015) PeerJ Computer Science, 1: e3). Using ZBrowse, we were able to view the SNPs for multiple traits simultaneously and plot those traits over time so that we could determine the DAP on which each SNP was significant and thus identify transient QTL peaks that turn “on/off” at specific DAP.

Candidate Gene Identification

The sorghum reference genome assembly version 3.1 was used to identify genes colocalizing with or adjacent to the associated SNPs. A genome scan of 15 kb upstream and 15 kb downstream of each significant SNP was performed to identify candidate genes. Phytozome (https://phytozome.jgi.doe.gov/pzlportal.html) was used to analyze the functional annotation of the candidate genes and to identify putative homologs in other species. Analysis of polymorphisms within candidate genes was done using three diversity panels of resequencing data available for sorghum via Phytozome (Paterson, A. H., et al. (2009) Nature, 457(7229): 551: Mace. E. S., et al. (2013) Nature Communications. 4: 2320: McCormick, R. F., et al. (2018) The Plant Journal, 93(2): 338-354). VCFtools was used to generate final variant calls after merging the three diversity VCFs, based on a minor allele frequency cutoff of >0.05, as well as generate distinct VCFs for accessions specifically in this study (Danecek, P., et al. (2011) Bioinformatics, 27(15): 2156-2158). Variant effects were estimated using the SNPeff pipeline and the v 3.1 sorghum reference to assess the potential impact of sequence variants on annotated gene models (Cingolani, P., et al. (2012) Fly, 6(2): 80-92). Correlations of the potential effects of polymorphism were based on overlap between publicly available sequence data and BAP accessions with phenotype data from this study.

### Results

Population Structure and Kinship

In this study, 369 accessions of the BAP grown under early cold stress conditions were evaluated. The BAP (Brenton, Z. W., et al. (2016) Genetics, 204(1): 21-33) comprises six subpopulations that represent a racial, geographic and phenotypically diverse selection of sorghum accessions. The BAP was designed to limit variation in key bioenergy traits, such as height and sensitivity to photoperiod, within a range of desirable values (Brenton, Z. W., et al. (2016) Genetics, 204(1): 21-33) (S1). A kinship matrix showing the genetic correlations in the BAP was generated (S2). The kinship matrix revealed distinct groups of highly correlated accessions. In many of the highly correlated clusters, the individual accessions shared characteristics such as country of origin and similar photoperiod sensitivity. For example, in one cluster, 47 of 48 accessions originated from Ethiopia. Another cluster consisted of 29 accessions, and 25 of those originated from Ethiopia, while 26 were photoperiod-sensitive. One cluster was comprised entirely of photoperiod-insensitive accessions, while 29 of 30 accessions in another cluster were of the cellulosic type. These clusters reflect an uneven population distribution and illustrate the necessity for controlling for population structure in this study.

Germination and Seedling Vigor

Seeds were sown in pots at 15° C. and grown at that temperature for 31 days. The temperature was then increased to 24° C. for seven days and then increased to 32° C. for the final 18 days of the experiment. Plant growth and development were observed using daily, non-destructive imaging throughout eight weeks. Germination dates were determined for each plant by image analysis and manual validation. At least two plants per genotype germinated for a total of 369 unique accessions. Of the 369 accessions, 41 germinated within ten days after planting (DAP) during the 15° C. cold treatment, and the remaining accessions germinated between 11 DAP and 46 DAP (S3). Accessions that failed to germinate or died by 46 DAP were excluded, leaving 309 accessions for subsequent phenotypic and genetic analyses (S3).

Trait Heritability and Phenotypic Variation

To analyze the phenotypic response to early cold stress in the BAP, we evaluated five bioenergy-related traits of interest: biomass, height, hull area, water use efficiency (WUE), and relative growth rate (RGR). Images captured daily from three angles and the amount of water each plant received daily enabled calculation of phenotypic measurements for each plant for each day of the experiment.

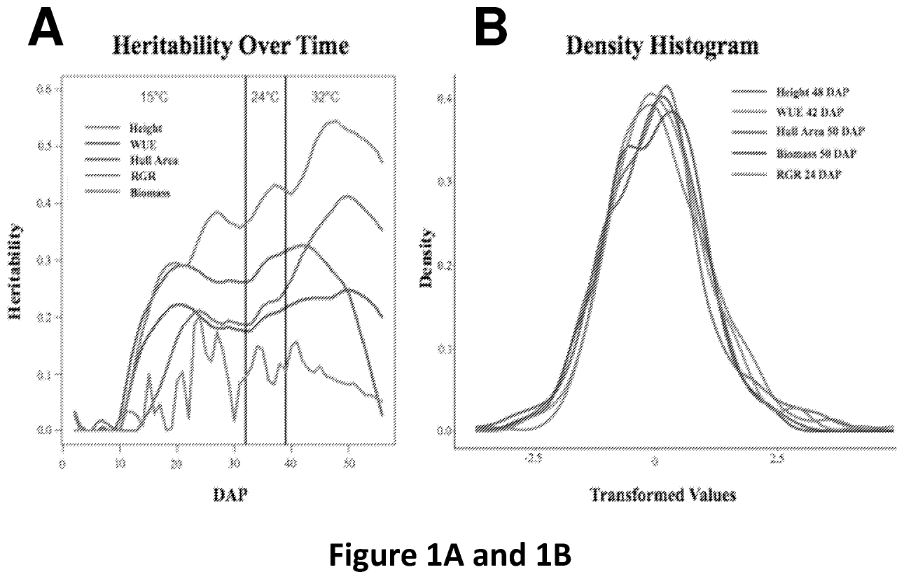

We used the three replicates for each accession to calculate broad-sense heritability within the experiment for each of the traits at each DAP to determine the proportion of phenotypic variation explained by genetic variation in the BAP. The heritability for each trait changed over time and as the temperature increased (FIG. 1A), generally increasing through the early growth period and decreasing as the plants became larger and more root bound. Heritability estimates ranged from 0.544 to 0 (S4). Height at 44-54 DAP was the most heritable trait with a heritability range of 0.544 to 0.503. The heritability values were moderate (0.493 to 0.3) for height at 23-43 DAP, height at 55-56 DAP, WUE at 36-45 DAP, and hull area at 42-56 DAP. The remaining traits had low heritability (0.297-0).

Heritability of WUE increased between 10-20 DAP, remained relatively flat until 42 DAP, then decreased. The decreased heritability of WUE was expected and coincides with an increased uncertainty of biomass calculations and water use estimates during the final two weeks of the experiment. There was a significant increase in overlapping of leaves after 42 DAP, resulting in the underestimation of plant biomass from the imaging data. The heritability for RGR had no discernable trend. The range of variation observed for the phenotypes of interest is depicted in the density histogram (FIG. 1B). The density histogram shows the frequency of the mean of the analyzed phenotypes at the DAP at which each trait had the highest heritability.

Trait Correlations

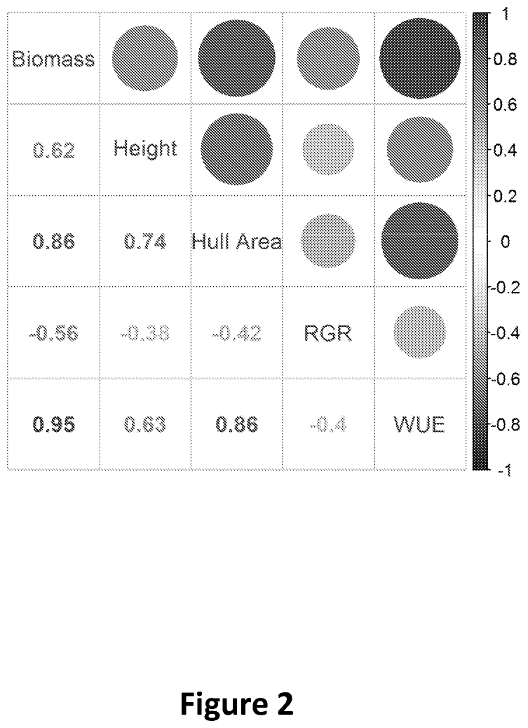

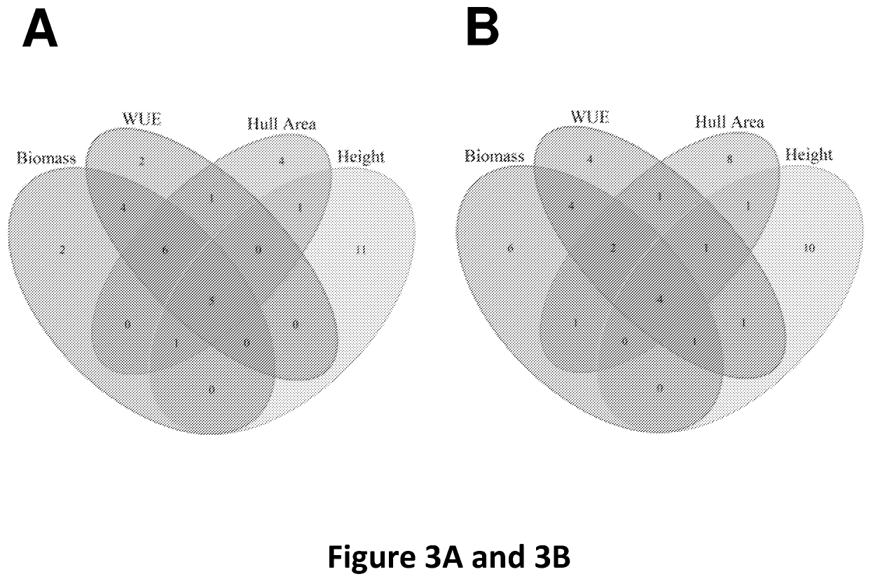

The correlations among biomass, height, hull area, RGR, and WUE were evaluated at 51 DAP (FIG. 2). The plants began to overlap and grow outside of the camera view after that date, making pixel numbers acquired for area measurements less indicative of the actual plant size. Biomass was most highly correlated to WUE with a r² of 0.95 but also showed high correlations to hull area and height. The correlation between biomass and RGR was negative with a r² of −0.52.

Phenotypic Ranking of Accessions

The daily plant images were analyzed to quantify the biomass, hull area, height, and WUE phenotypes for each DAP. Based on the resulting values, for each trait, we ranked the accessions based on their performance. Some accessions were ranked high or low for multiple growth-related traits. The Venn diagram (FIG. 3) illustrates overlaps among the top or bottom 5% of accessions at 51 DAP. Figure provides a complete ranking of accessions for each trait at 51 DAP. Five accessions (PI 152771, PI55064, PI514456, PI 569454, and PI534165) are represented in the bottom-ranked accessions for all four traits. PI154988, PI535794, PI569148, PI535792, PI329584, and PI535793 are among the lowest ranked accessions for biomass, hull area, and WUE, while PI569457 is among the lowest accessions for biomass, hull area, and height. Six additional accessions ranked as poor performers for two traits of interest: PI452544, PI552851, 570038, and PI570254 for biomass and WUE; PI157035 for hull area and height; and PI540518 for hull area and WUE. Among the top-ranked accessions, PI19770, PI329299, PI455301, and PI453106 were in the top 5% for biomass, height, hull area, and WUE. PI329403 and PI452619 were among the top accessions for biomass, hull area, and WUE, while PI92270 was a top performer for hull area, height, and WUE and PI563222 was among the top lines for biomass, height, and WUE. Finally, eight additional lines ranked among the top accessions for two traits.

Ideotype-Positive and Ideotype-Negative Accessions

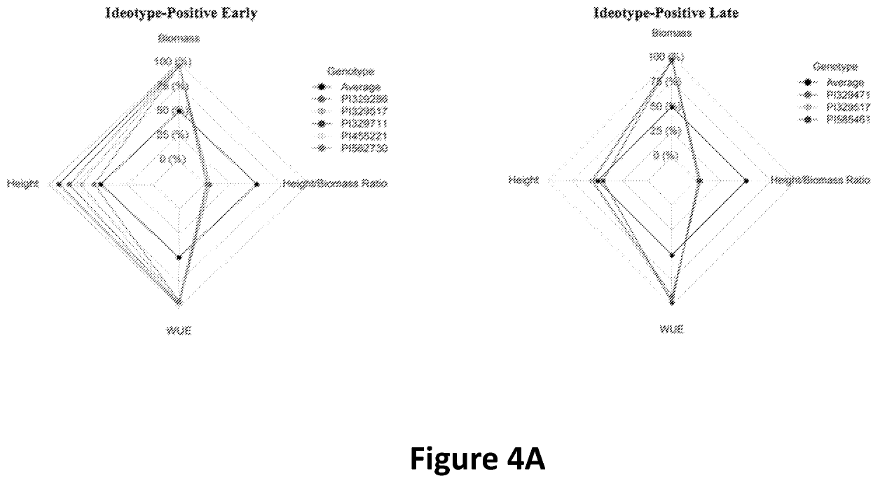

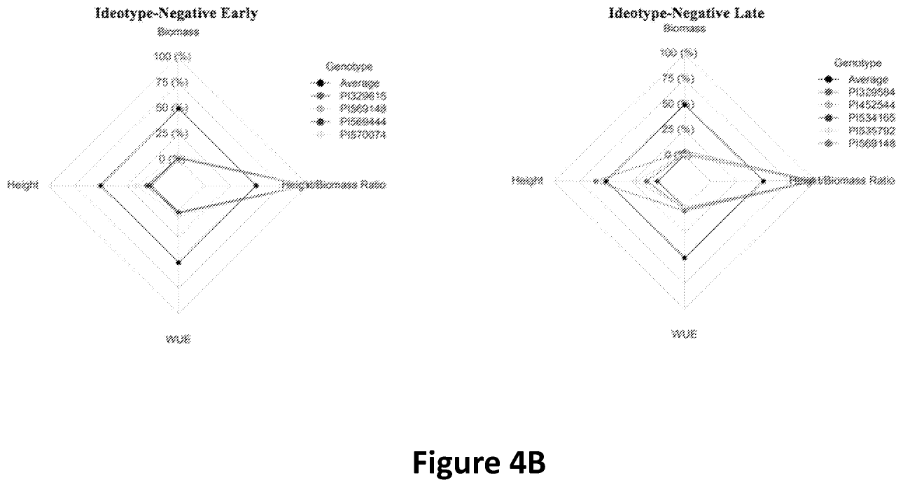

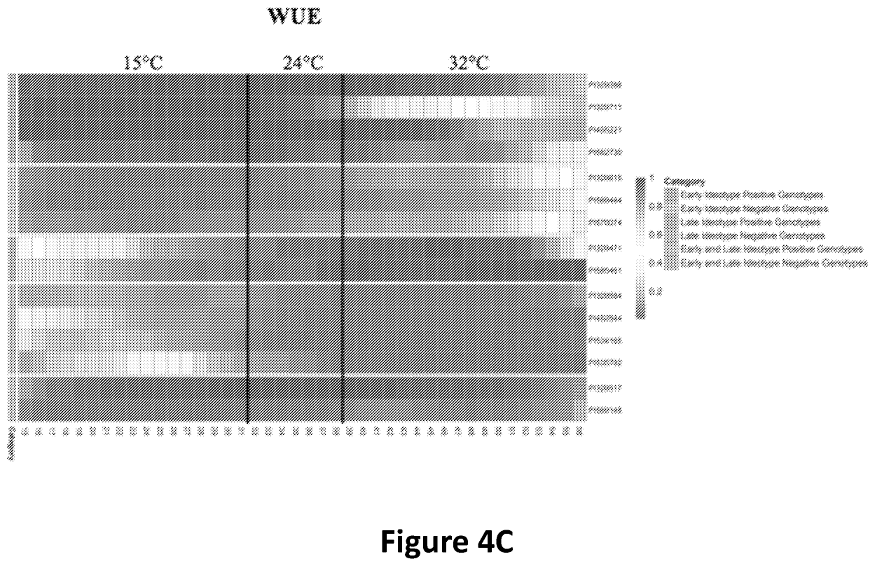

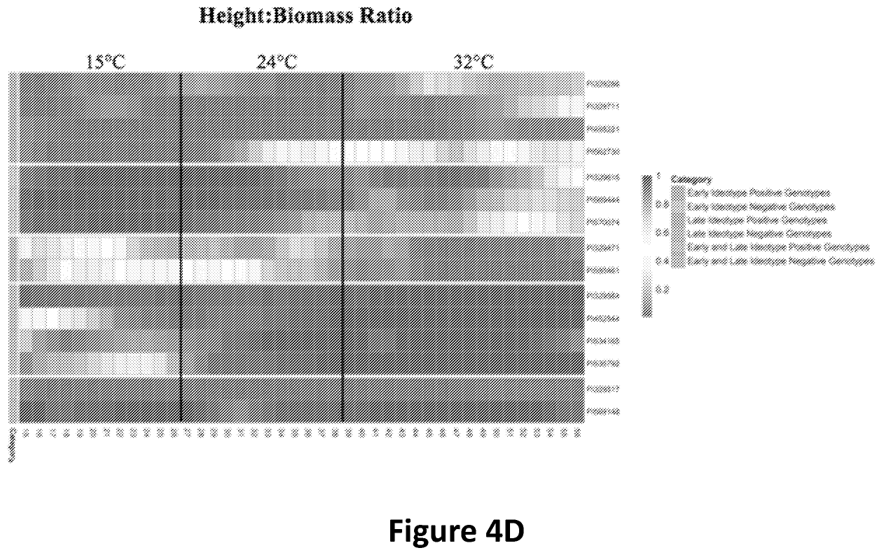

Comparisons of accession rankings among multiple traits facilitated the identification of ‘ideotype-positive’ and ‘ideotype-negative’ accessions. Accessions ranked within the top 10% for WUE but with a height-to-biomass ratio in the bottom 5% of accessions were designated as ideotype-positive (FIG. 4A). There were five ideotype-positive accessions in the early period of the experiment, from 15 DAP to 31 DAP at 15° C.: PI329286, PI329517, PI329711, PI455221, and PI562730. There were three ideotype-positive accessions in the late period of the experiment, from 39 DAP to 56 DAP after the temperature had been increased to 32° C.: PI329471, PI329517, and PI585461. Accession PI329517 was ideotype-positive throughout the experiment.

Accessions within the bottom 10% for WUE accessions but with a height-to-biomass ratio in the top 5% of accessions were designated as ideotype-negative (FIG. 4B). There were four early ideotype-negative accessions: PI329615, PI569444, PI569148, and PI570074, and five late ideotype-negative accessions: PI329584, PI452544, PI534165, PI535792, PI569148. Accession PI569148 maintained an ideotype-negative phenotype throughout both the 15-31 DAP and 39-56 DAP periods. Heatmaps show the WUE (FIG. 4C) and height-to-biomass ratios (FIG. 4D) of the early and late ideotype-positive and ideotype-negative accessions from 15 DAP through 56 DAP.

Temporal WUE Profiles and Clustering of Accessions

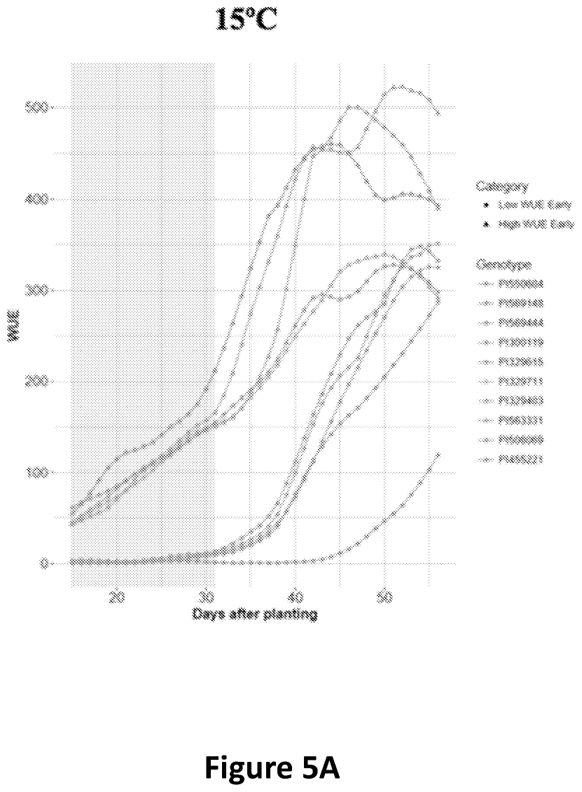

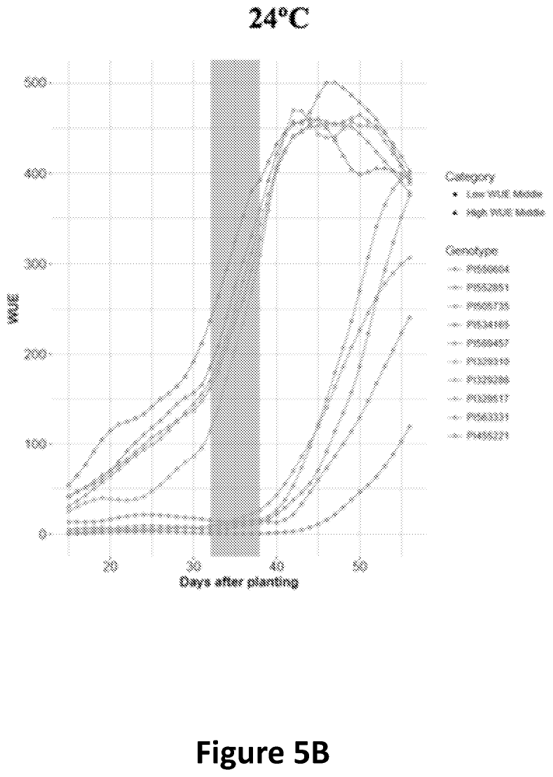

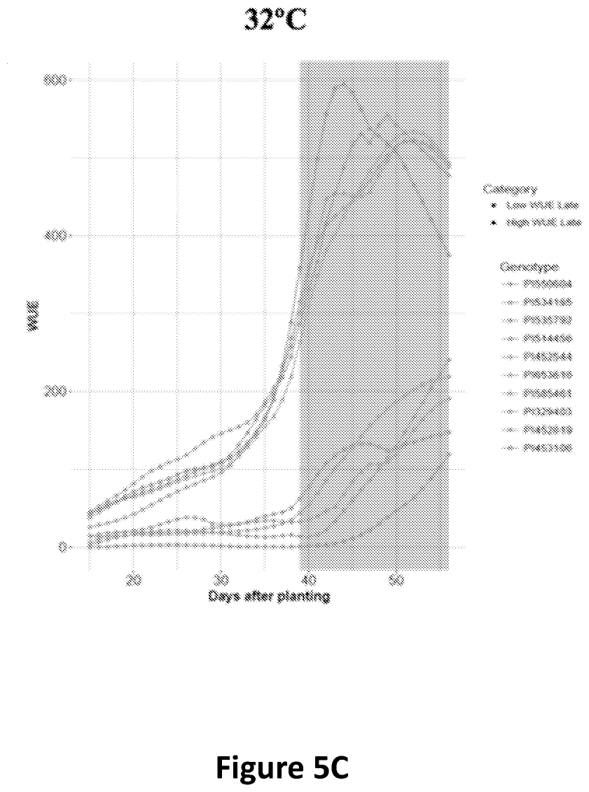

The daily quantification of traits provided the opportunity to assess temporal changes in trait values as the temperature increased and plants developed. For brevity, we only refer to the five top and bottom-ranked accessions for WUE, a key trait. FIG. 5 shows the temporal WUE profiles for the five highest- and lowest-ranked accessions at each temperature stage. The top-ranked accessions for WUE at each temperature exhibited a WUE profile over time that is distinct from the profiles of the accessions ranked lowest for WUE accessions, and the separation became more apparent as the plants developed and the temperature increased (FIG. 5). Even at 15° C., the highest ranked accessions for WUE are distinguishable with a steeper increase in WUE over time compared to the bottom-ranked accessions. Some accessions were ranked high or low for WUE at one temperature stage only, whereas others were ranked high or low for WUE across temperatures. For example, PI550604 was among the bottom-ranked accessions for WUE at each temperature, and PI534165 was among the bottom-ranked at 24° C. and 32° C. PI455221 was a top-ranked accession for WUE at both 15° C. and 24° C., while PI329403 was among the highest ranked accessions at 15° C. and 32° C.

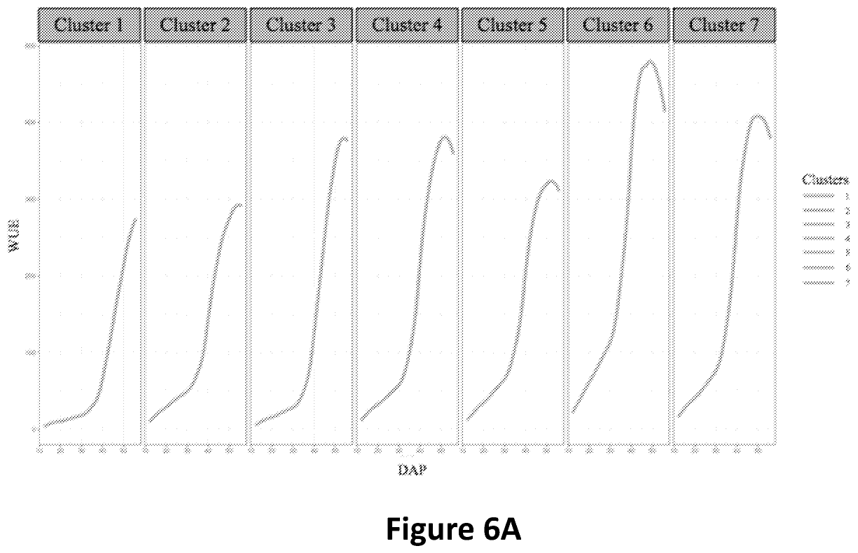

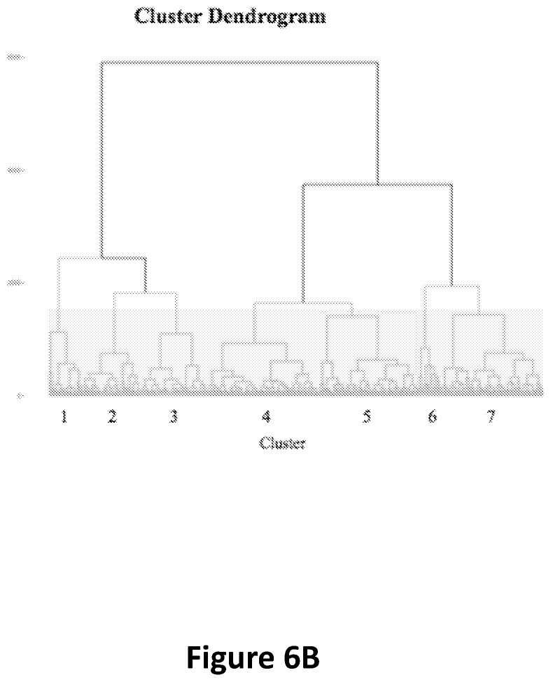

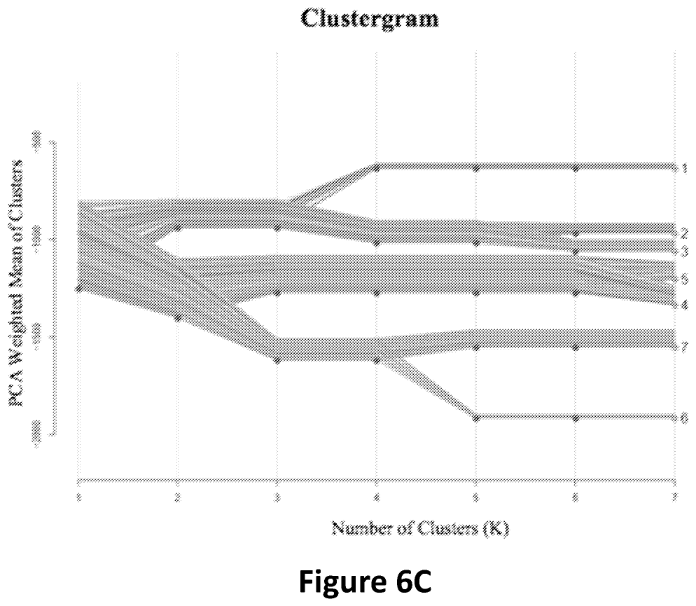

For each accession, we analyzed the profile of each phenotype over time and at different temperatures. Again, for brevity, here we only refer to WUE. For example, the temporal WUE profiles for the BAP accessions separate into seven clusters, each represented by a growth curve represented by the average of all accessions in that cluster (FIG. 6A). The temporal WUE profile for each of the seven clusters is distinct. The mean WUE for accessions that comprise Cluster 1 is low at 15° C., shows a minimal increase at 24° C., and increases steadily at 32° C., but never reaches levels as high as several other clusters. Cluster 2 shows a slightly higher WUE at 15° C. compared to Cluster 1 and an increase in WUE at 24° C. and 32° C., but never exceeds moderate levels of WUE. In Cluster 3 there is little increase in WUE until the temperature increases to 32° C. and this cluster eventually reaches moderate WUE. WUE for Cluster 4 is low at 15° C., increases at 24° C. and 32° C. Cluster 5 exhibits a moderate increase in WUE at 15° C., a rapid increase at 24° C., but WUE increases slowly at 32° C. Cluster 6 has an early and robust increase in WUE across all three temperatures in the experiment and reaches the maximum peak WUE of all seven clusters. Finally, WUE for Cluster 7 is moderate at 15° C. and increases steadily at 24° C. and 32° C., but ultimately peaks at a moderate level. The dendrogram tree (FIG. 6B) depicts the hierarchical clustering of the BAP accessions into seven groups based on their temporal WUE profiles, with each cluster represented by a different color. The clustergram of the PCA-weighted mean (FIG. 6C) illustrates the divergence of the temporal WUE profiles of the BAP accessions into the seven stable clusters which captured the maximum phenotypic difference for this particular trait.

Genome-Wide Association Study

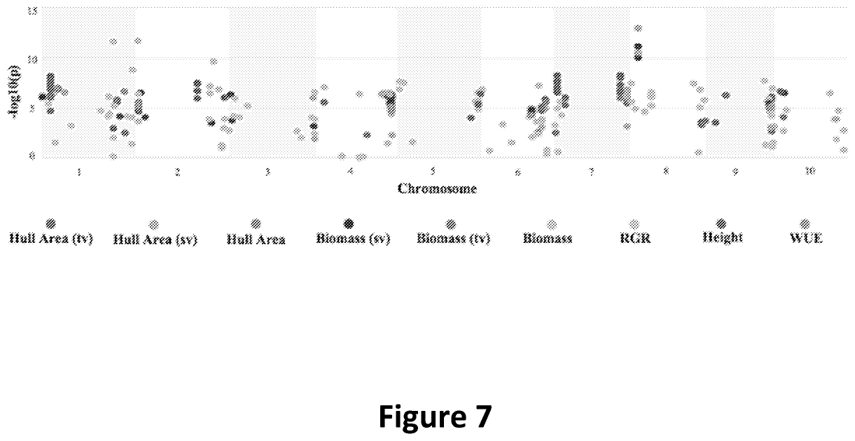

A genome-wide association study (GWAS) was performed to characterize the genetic architecture underlying response to early cold stress conditions in the BAP. GWAS was performed using the multi-locus mixed model (MLMM) algorithm (Segura. V., et al. (2012) Nature Genetics, 44(7): 825). MLMM uses multiple loci in the model to yield a higher detection power and lower the potential of false discoveries. FIG. 7 illustrates the detection of highly significant signals related to each trait of interest on all ten sorghum chromosomes. Each significant SNP has a maximum corrected p-value of 0.05. This analysis identified a total of 2,305 highly significant SNPs associated with biomass, height, hull area, WUE, and RGR.

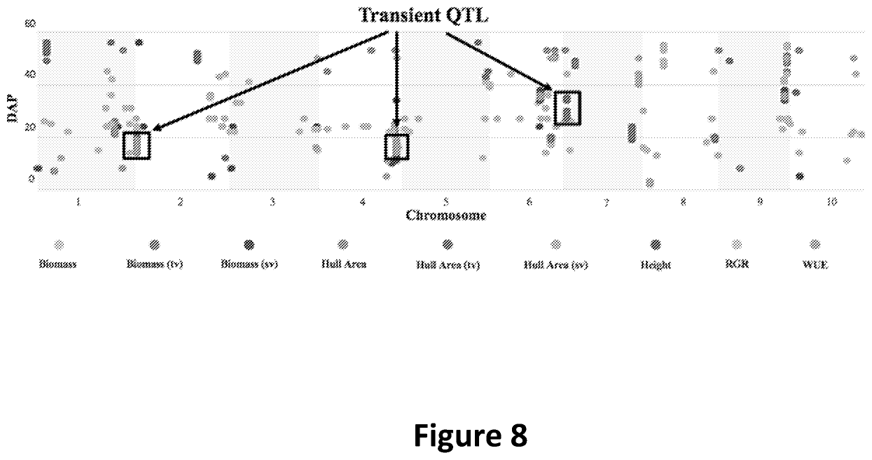

In order to identify SNPs significantly correlated with phenotypes at specific DAP and correlated with temperature changes, we also used the values for each phenotype at each DAP (FIG. 8) for the GWAS.

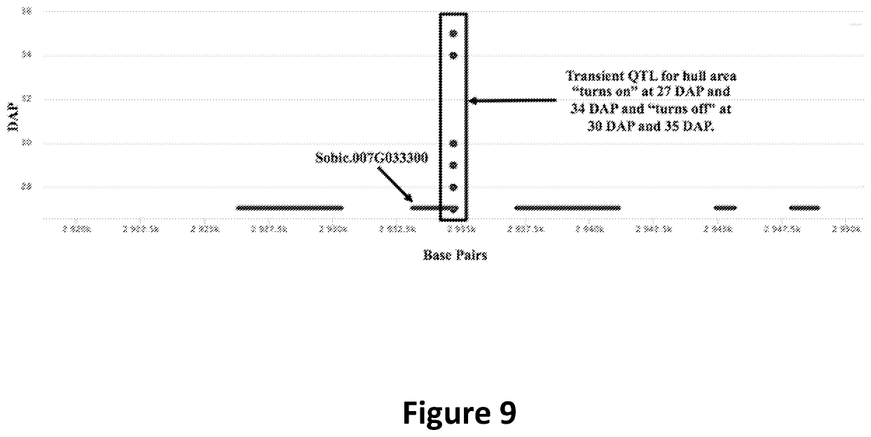

This analysis revealed several QTL that displayed a transient response, appearing at specific times and developmental stages, and as temperatures increased. (FIG. 8). For example, the highly significant hull area SNP 7:2,934,702 was transiently detected from 27-30 DAP and again from 34-35 DAP (FIG. 9).

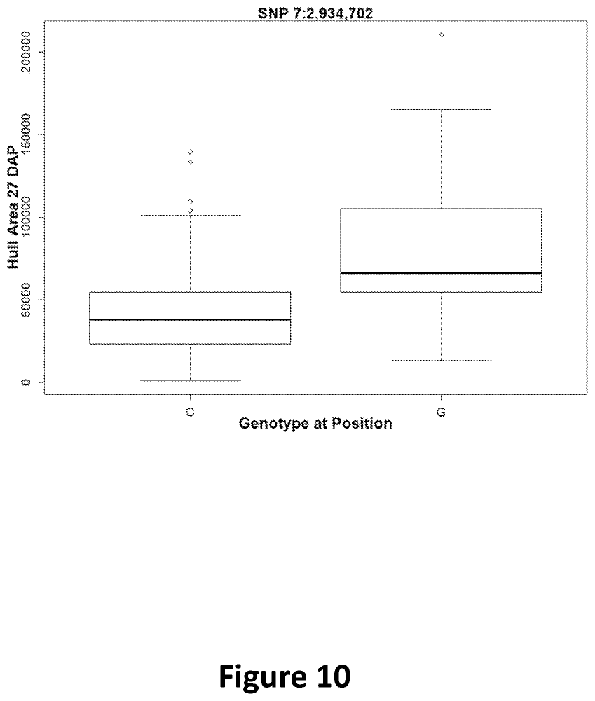

For the transient QTL that were detected, we also examined the phenotypic differences for the alternative alleles at a particular SNP. For the hull area SNP 7:2,934,702 shown in FIG. 9, allele boxplots (FIG. 10, S7) shows that the presence of a “G” at the position corresponds to a significantly higher value for hull area than a “C” for 27-30 DAP and 34-35 DAP. SNP 7:2,934,702, located within gene Sobic.007G033300, encodes a plastocyanin-like protein. Plastocyanins are copper-containing chloroplast-localized proteins involved in photosynthesis and function in electron transfer between photosystem II and photosystem I (Sigfridsson, K. (1998) Photosynthesis Research, 57(1): 1-28). Ibis putative plastocyanin, Sobic.007G033300, is highly expressed in sorghum shoots, leaves and early inflorescences (on the world wide web at phytozome.jgi.doe.gov/pz/portal.html) (Makita, Y., et al. (2014) Plant and Cell Physiology, 56(1): e6-e6). We examined other sequence variation existing within this gene using public re-sequencing data and found a total of seven SNP variants resulting in nonsynonymous (NS) amino acid substitutions as well as an additional inframe three basepair deletion segregating within individuals in the BAP (S8a). A total of 80 of the phenotyped BAP accessions contain no variants in Sobic.007G033300 relative to the reference sequence, while the remaining accessions contain between one to eight of the variants summarized in S8a. When grouped, individuals carrying at least one variant in Sobic.007G033300 have a significantly larger hull area from 27 DAP in comparison to accessions containing no variants (S8b).

The GWAS also identified pleiotropic QTL. A transient QTL on chromosome two (SNP 2:1,199,928) was significant for WUE and biomass from 14-19 DAP. SNP 4:64256235 on chromosome four was a significant transient QTL for WUE and biomass from 14-19 DAP, as well as height at 34 DAP. Transient QTLs on chromosomes six and nine (SNP 6:42,116,590 and SNP 9:55067080, respectively) were significant for WUE and biomass at 35-36 DAP. S9 shows the traits and DAP associated with each SNP.

Candidate Gene Identification

We conducted a genome scan of the 15 kb upstream and 15 kb downstream of each significant SNP in order to identify candidate genes. Functional annotations, putative homologs, and polymorphisms within the candidate genes based on public genomic resequencing data were analyzed using Phytozyme (https://phytozome.jgi.doe.gov/pz/portal.html). GWAS identified highly significant QTL near 72 candidate genes (S10) with putative functions potentially related to biomass, height, hull area, WUE, and RGR, and in response to cold. Many of these candidate genes are close to pleiotropic QTL implicated in cold stress response in addition to the biomass-associated phenotypes.

For brevity, here we will only refer to one a priori candidate gene. GWAS identified SNP 6:40,312,463 which was significantly associated with RGR at 33 DAP and is located within the gene Sobic.006G057866, which encodes PSEUDO-RESPONSE REGULATOR 7 (PRR37)/MATURITY 1 (Ma1) and is involved in flowering time regulation in sorghum (Murphy, R. L., et al. (2011) Proceedings of the National Academy of Sciences. 108(39): 16469-16474). SNP 6:40,312,463 causes a non-synonymous amino acid substitution (Asn184Lys) within Ma1, and although accessions carrying this polymorphism show a slightly reduced mean RGR at 33 DAP, they were not significantly different (S11a). This observation suggests that allelic variation at SNP 6:40,312,463 is not likely to be a robust contributor to phenotypic variation in RGR. However, interestingly, the majority of accessions including both BAP accessions as well as from other diversity panels (˜90%) carry a polymorphism in Ma1 resulting in loss of the annotated reference stop codon, which could drastically increase the protein size and affect its function within non-reference accessions (S11b).

### Example 2

Plastocyanin Gene Editing

As disclosed herein, the gene Sobic.007G033300 was identified in GWAS study of cold tolerance in sorghum, where a SNP in the gene was associated with hull area, among other phenotypes. Gene editing constructs were initiated to generate mutants in the gene in sorghum and the homologous gene (Sevir.6G069300) in the model plant Setaria viridis.

Four total constructs were built; two targeting the gene in sorghum and two targeting the gene in S. viridis, and targeting either the Exon 1 or the 3′ UTR for each species. Each construct contained two guide RNAs, spaced approximately 100 base pairs apart in the gene.

TABLE 2
Gene editing guide RNA (gRNA) sequences.

| | Target | | | SEQ ID |
|---|---|---|---|---|
| Construct | Species | Location | gRNA Sequence | NO: |
| pJA018 | S. viridis | Exon 1 | CCGGACGGGCTGTGGGACATGCA | 13 |
| pJA018 | S. viridis | Exon 1 | AAGACCAGGACCTTCCACCCCGG | 14 |
| pJA019 | S. viridis | 3′ UTR | CCCATAGCTTGTTTGTTAGACGC | 15 |
| pJA019 | S. viridis | 3′ UTR | CTTTTGTTATTTCAATACCTTGG | 16 |
| pJA020 | S. bicolor | Exon 1 | AGGCAAATTAATGAGAGGCTGGG | 17 |
| pJA020 | S. bicolor | Exon 1 | TACACGGTGGGCGCGCCCGCCGG | 18 |
| pJA021 | S. bicolor | 3′ UTR | CTGAGCCGCTGTAAGCTTACAGG | 19 |
| pJA021 | S. bicolor | 3′ UTR | ATGCATTATGAATGGCCAGATGG | 20 |

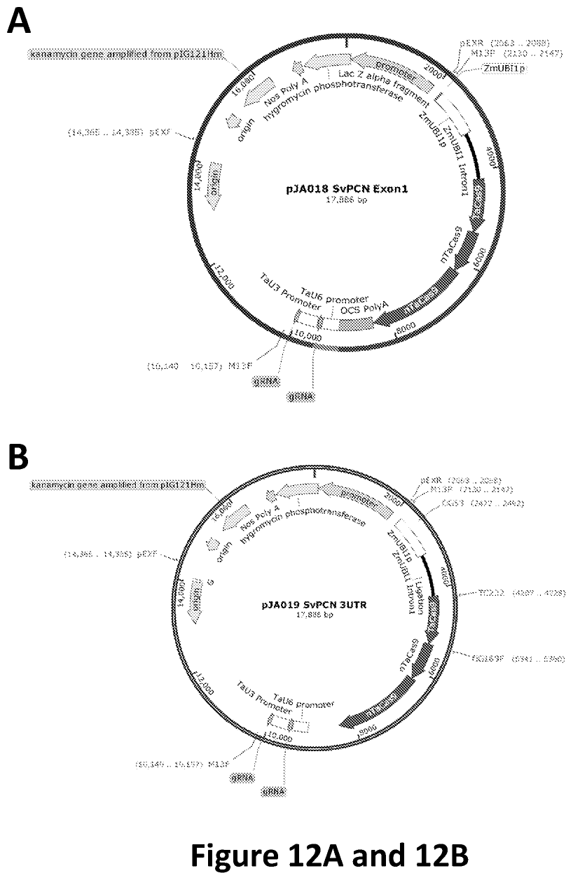

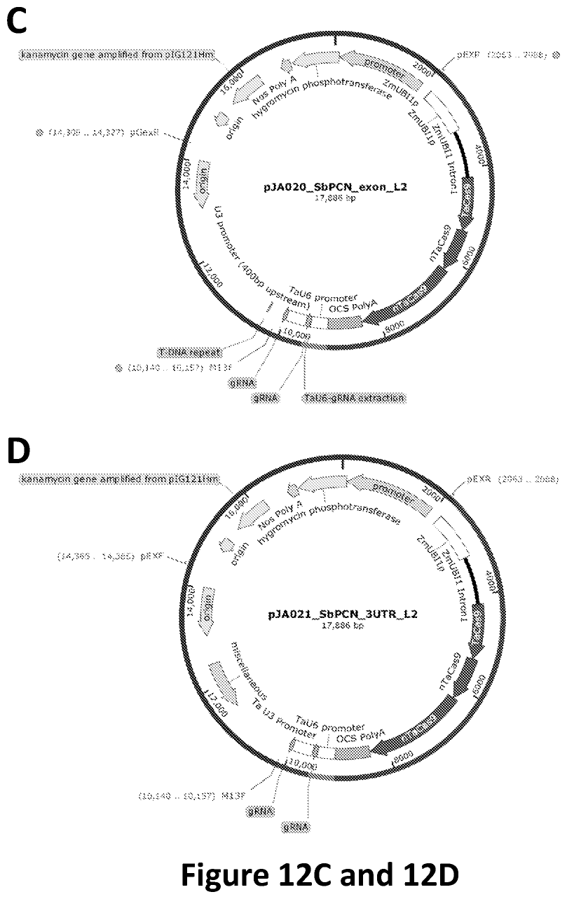

Constructs were generated to express the gRNAs under control of the wheat U3 or wheat U6 promoters, along with the wheat Cas9 gene, using a modular system developed in the Voytas lab. Plasmid maps for these CRISPR-Cas9 gene editing constructs are shown in FIG. 12.

The two Sobic.007G033300 CRISPR-Cas9 constructs targeting the sorghum plastocyanin gene were sent to Albert Kausch's group at Rhode Island for transformation.

For S. viridis transformation, plasmids were transformed into Agrobacterium strain AGL1, which was used by the DDPSC Plant Transformation Facility to transform the genotype ME34. For the exon-targeting construct, two events were returned. For the 3′ UTR construct, eight events were returned. TO plantlets were returned and grown to maturity in a growth chamber. T1 seeds were collected. DNA was isolated from the T1 and T2 generations for characterization of variant alleles. Two unique alleles were identified in Exon 1 and one identified in the 3′ UTR.

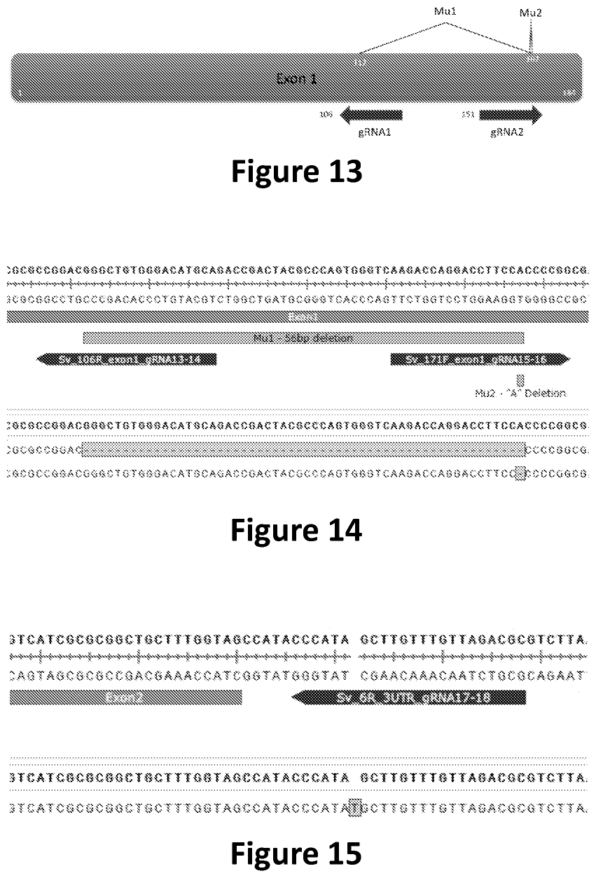

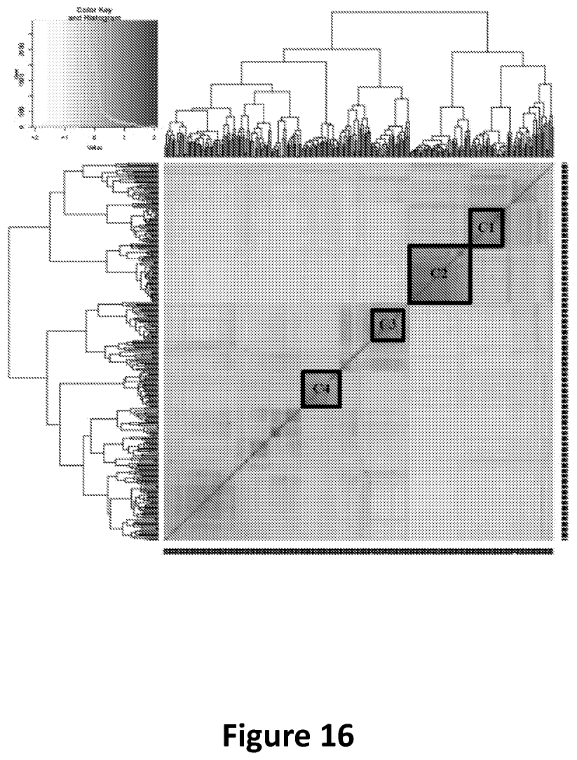

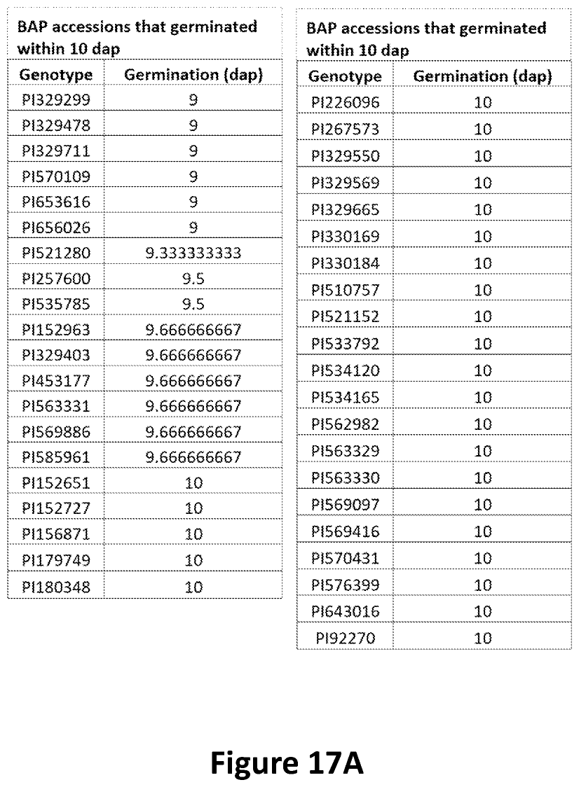

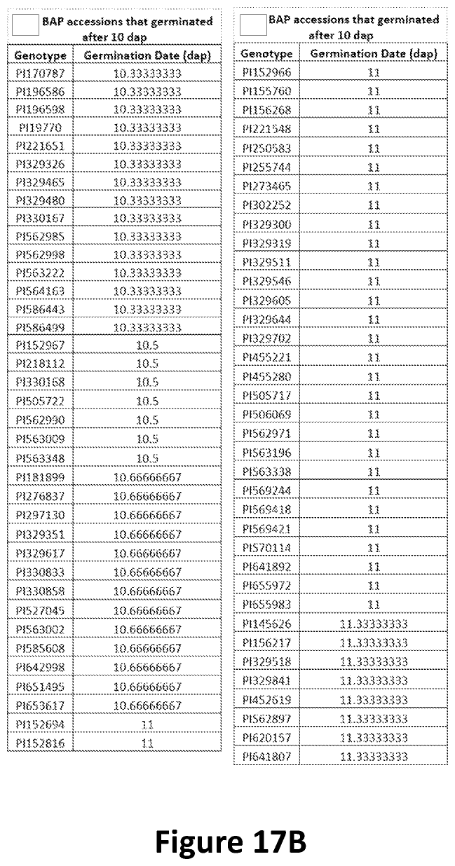

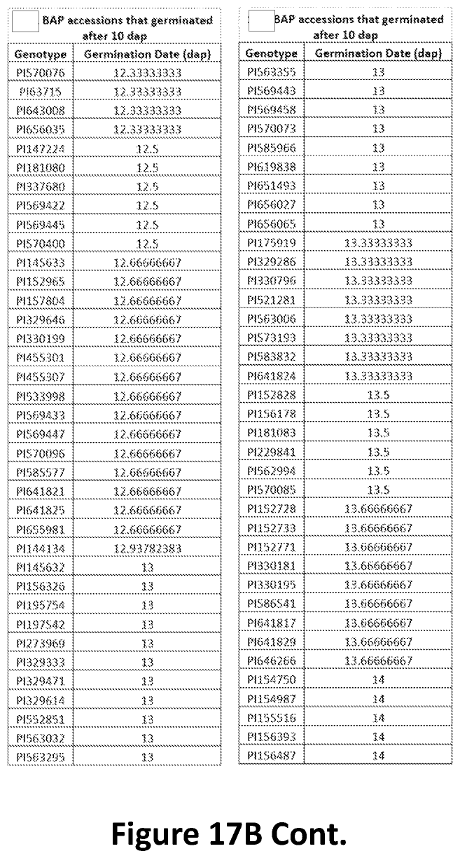

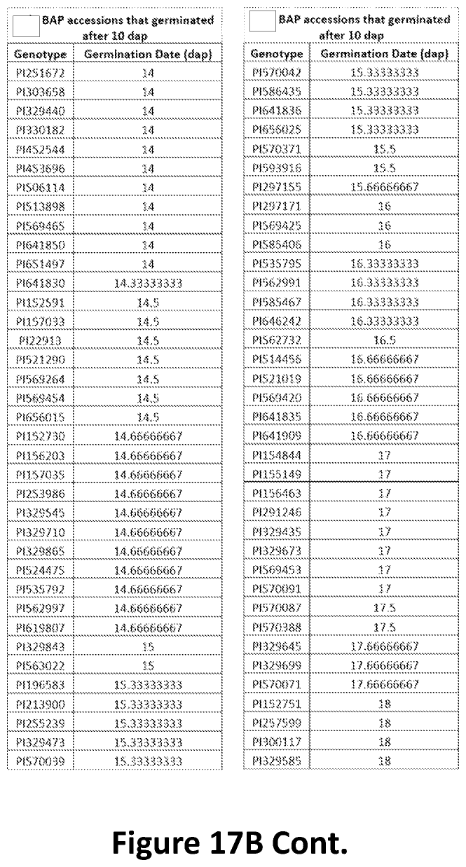

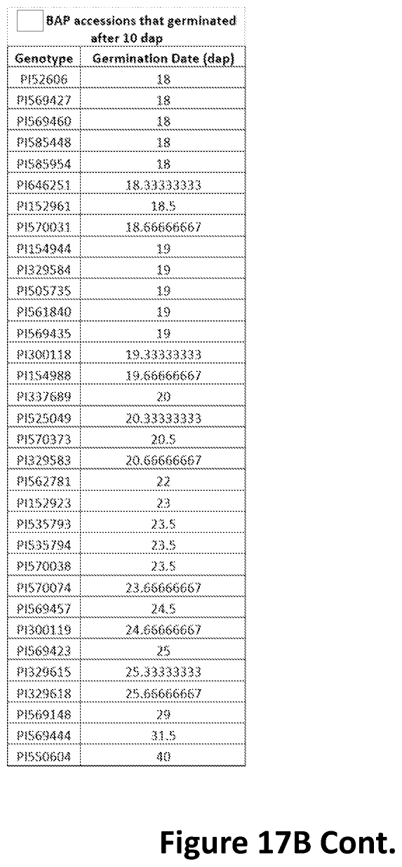

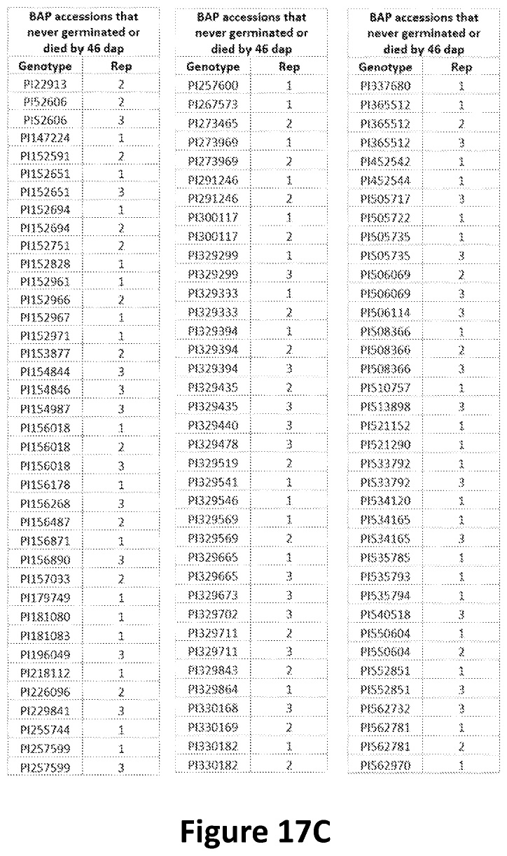

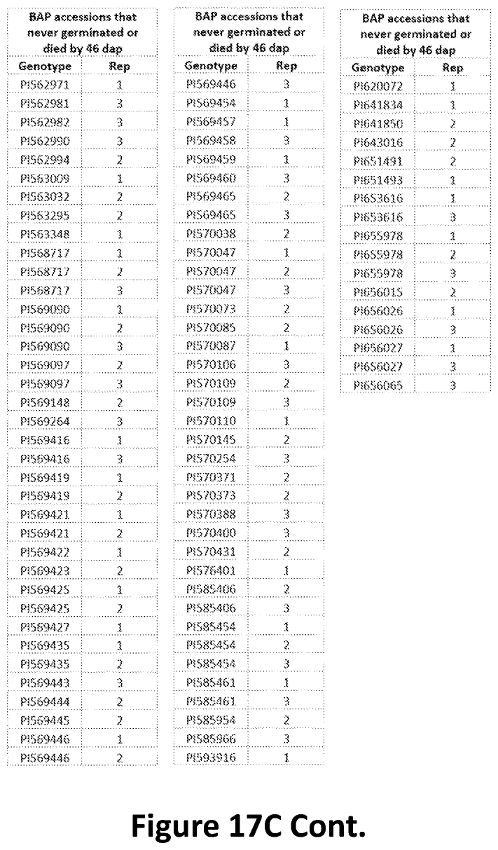

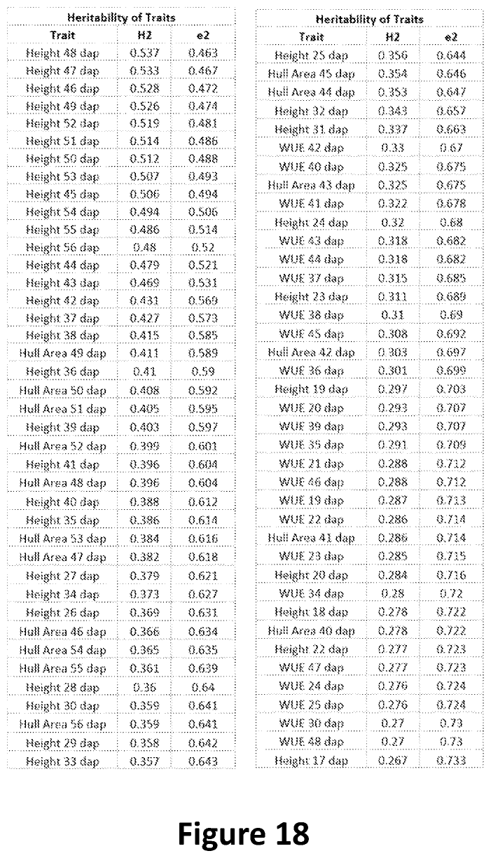

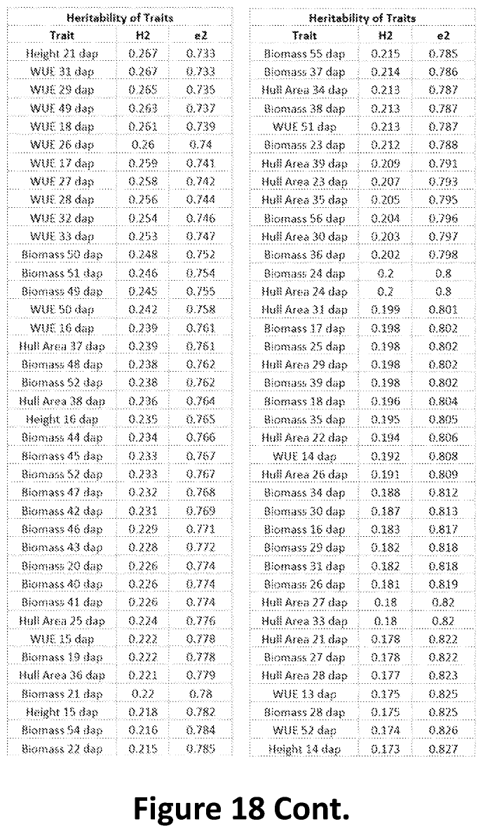

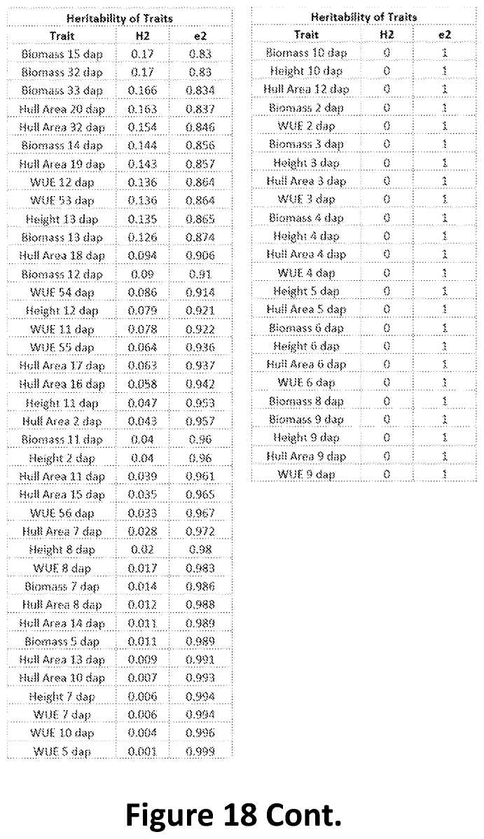

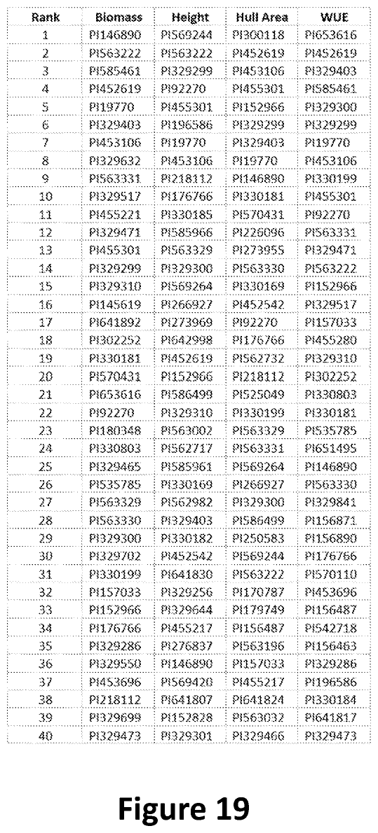

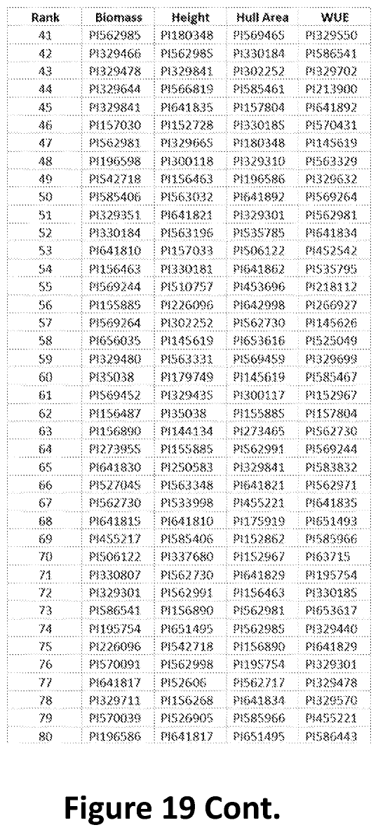

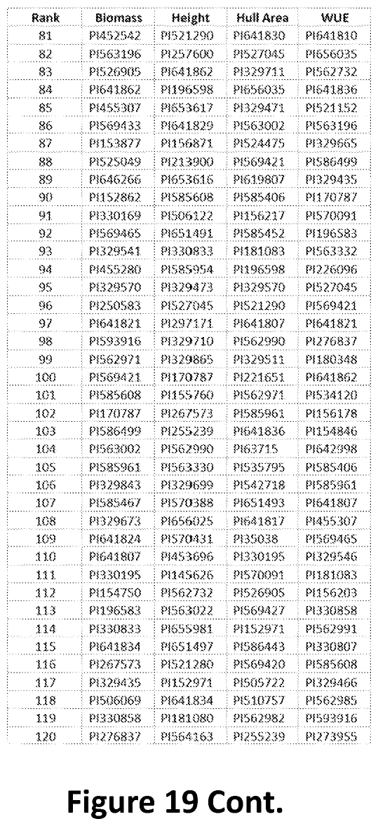

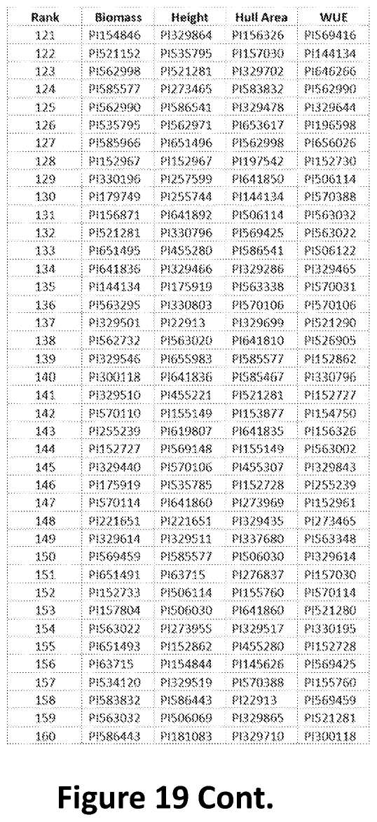

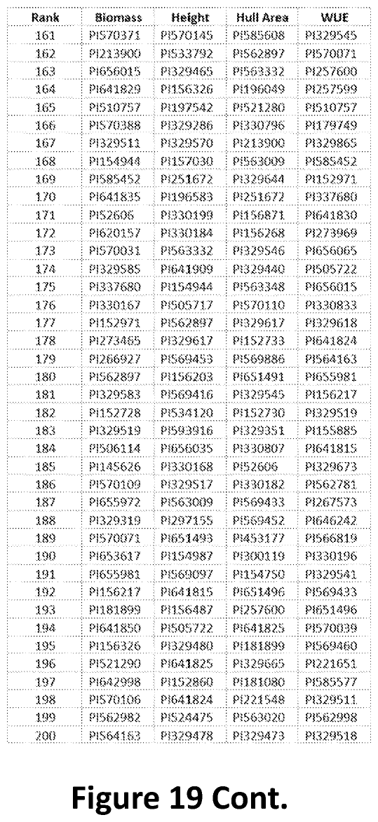

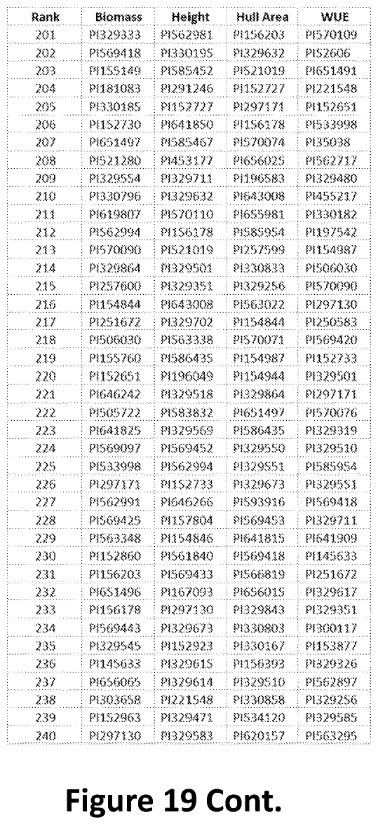

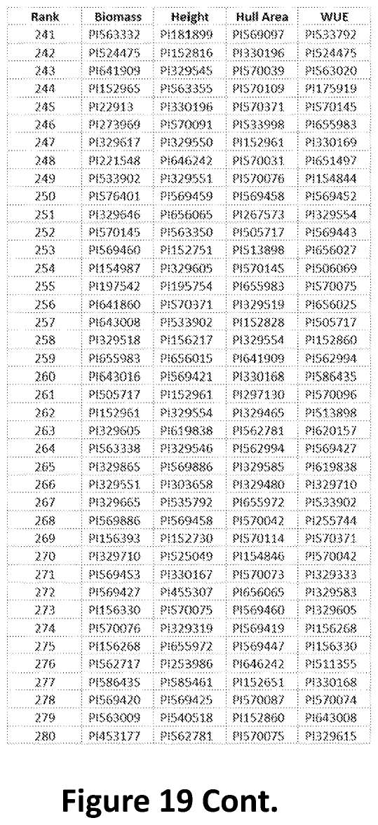

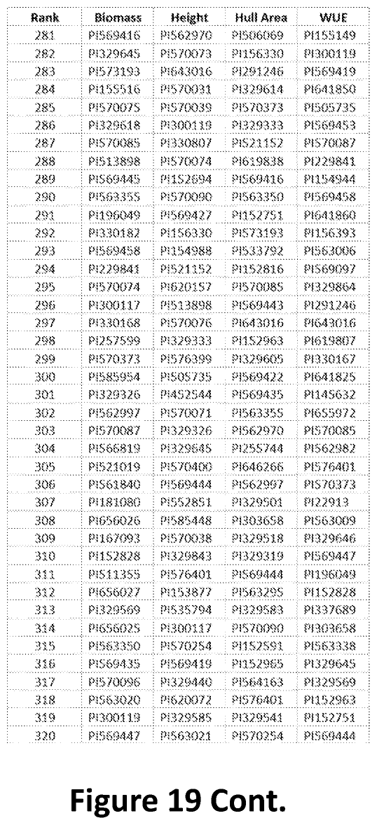

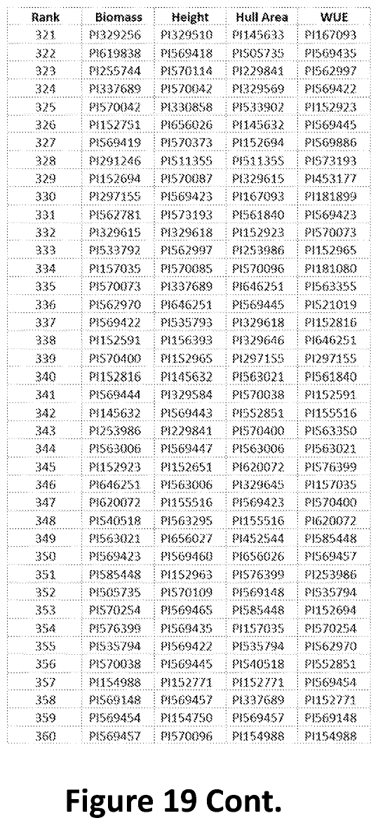

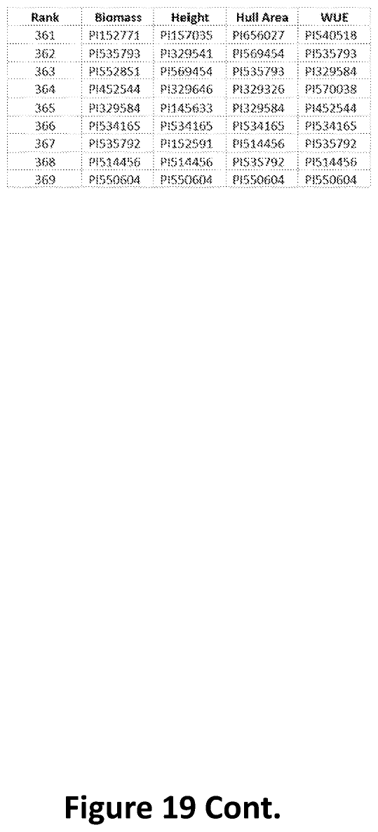

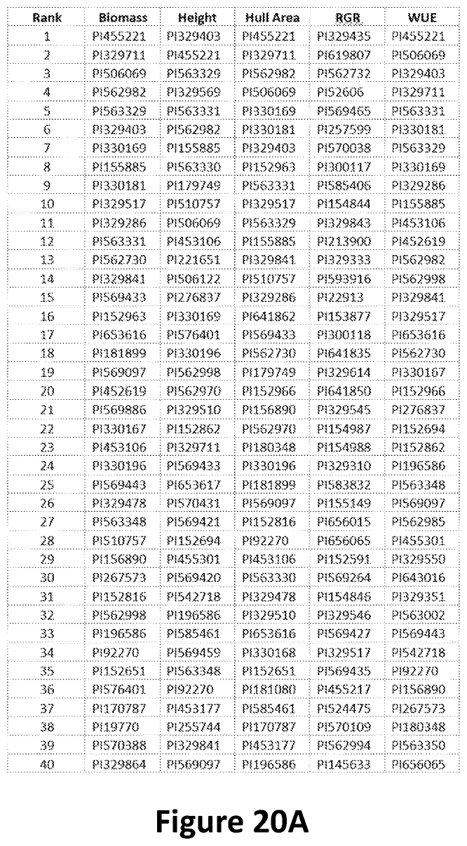

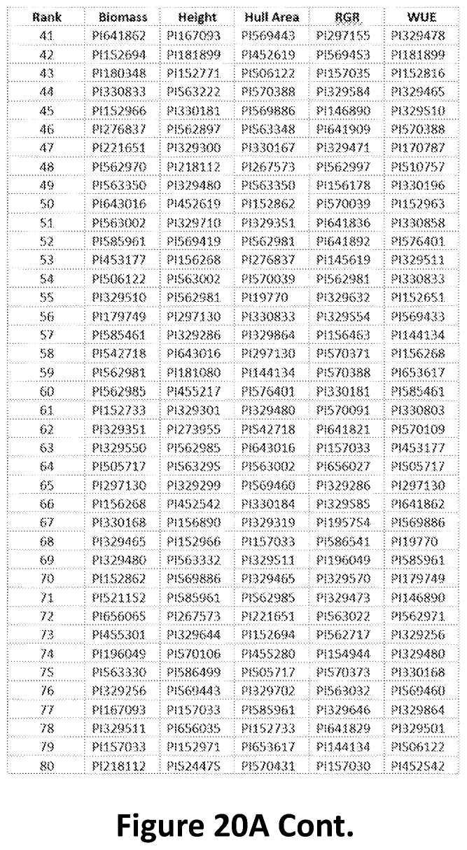

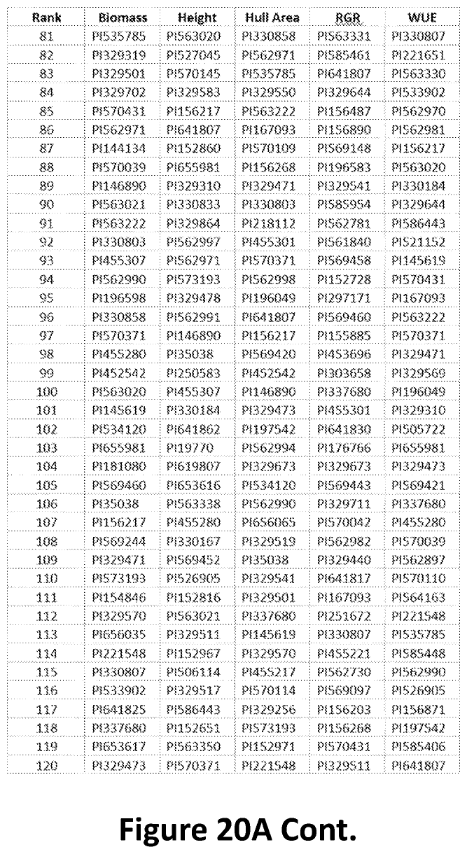

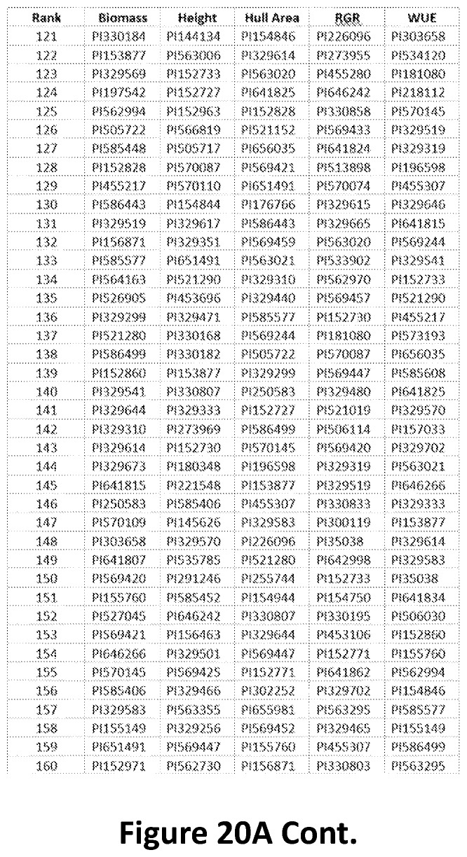

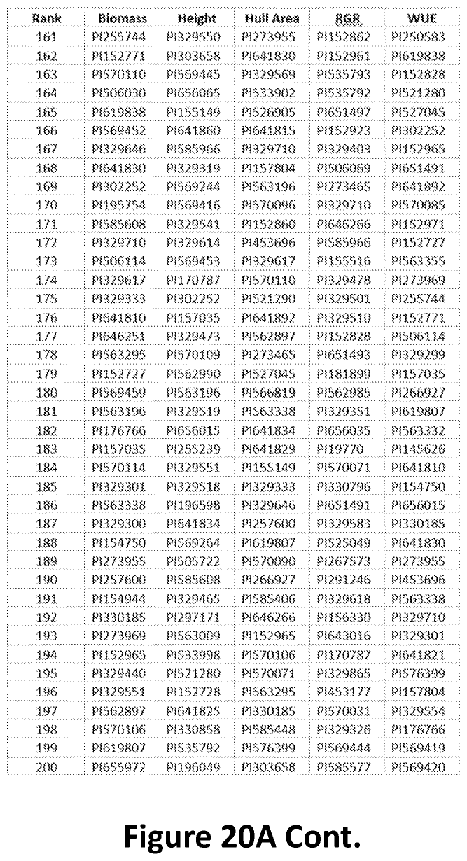

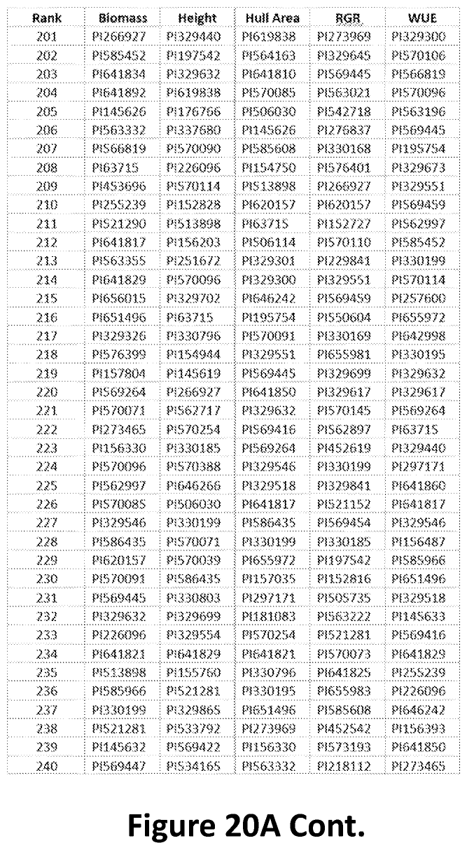

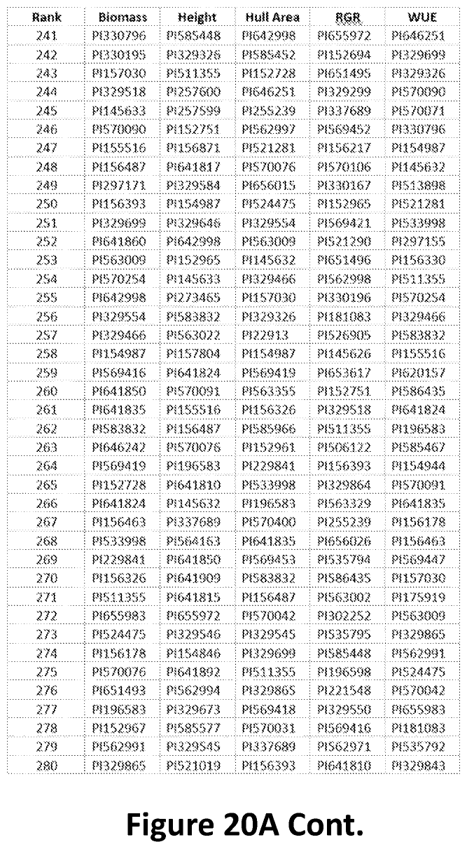

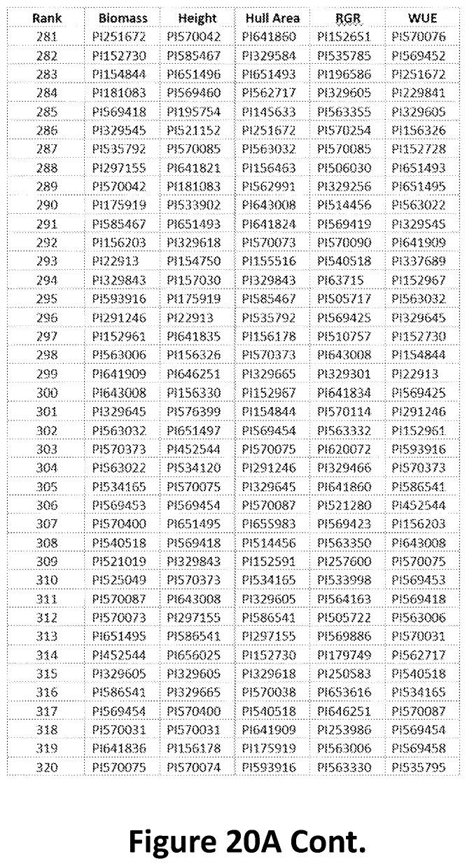

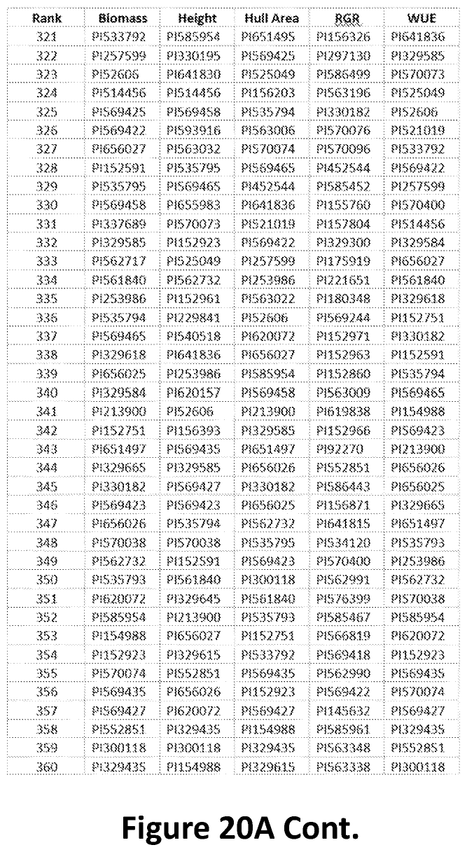

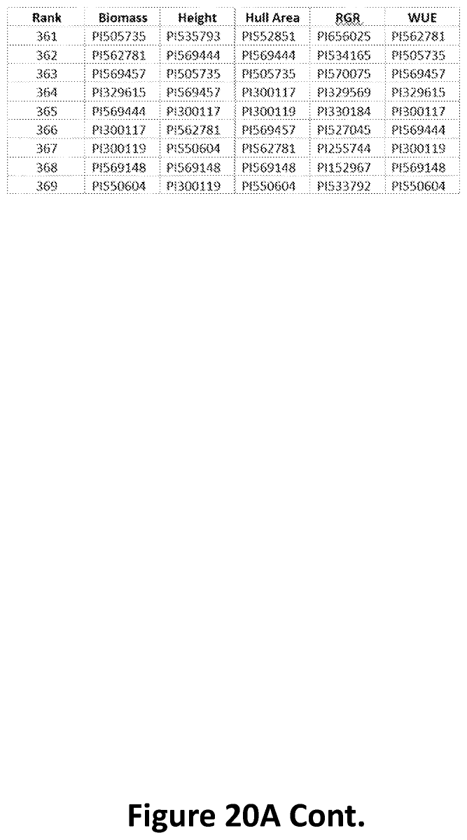

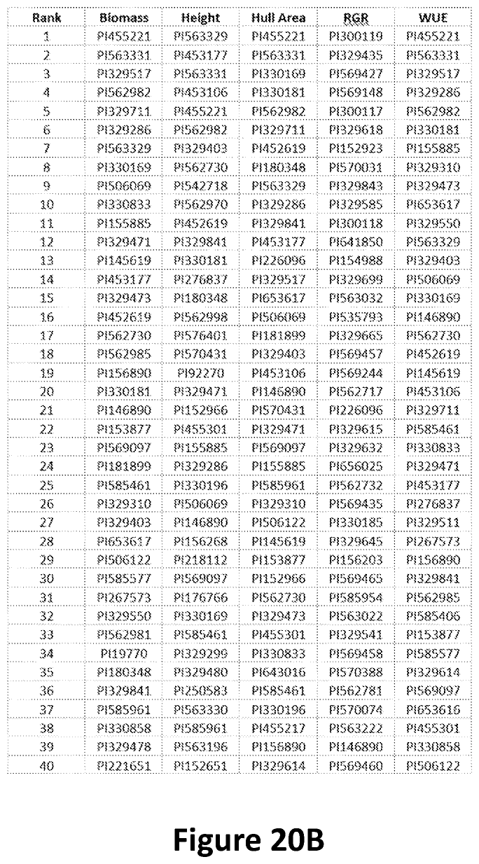

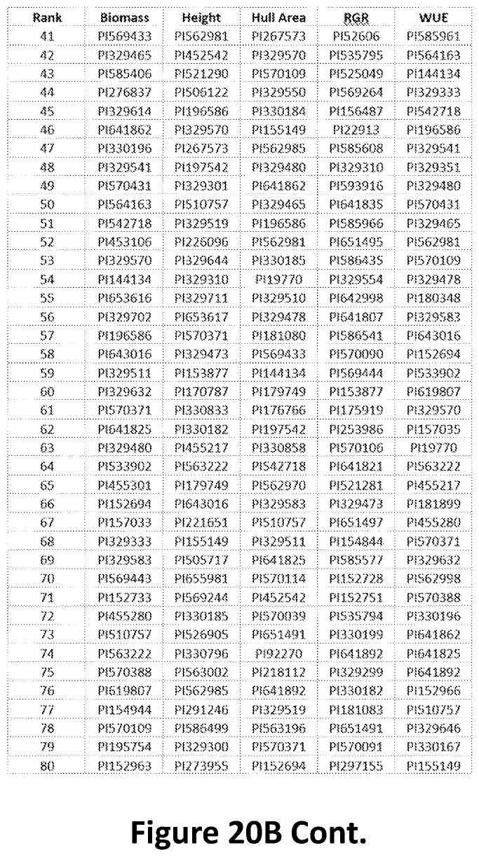

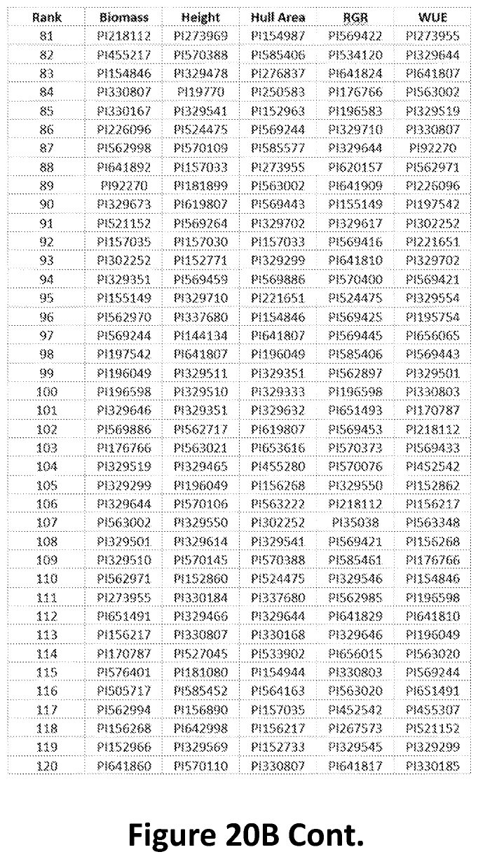

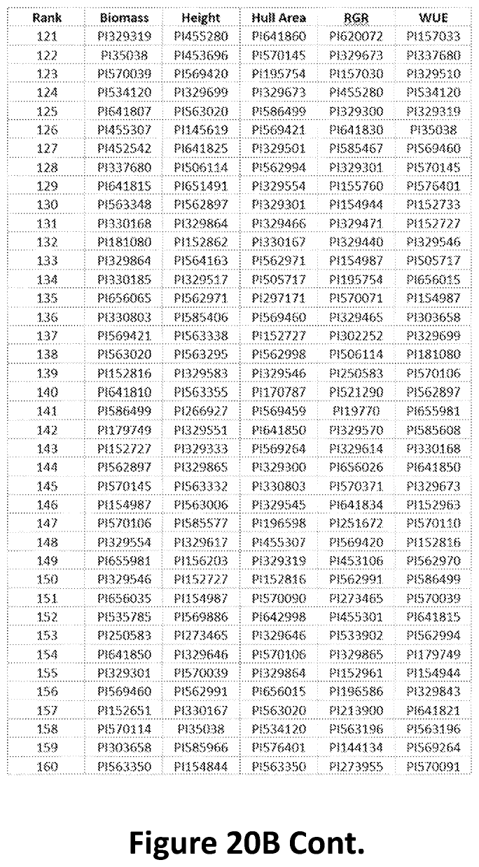

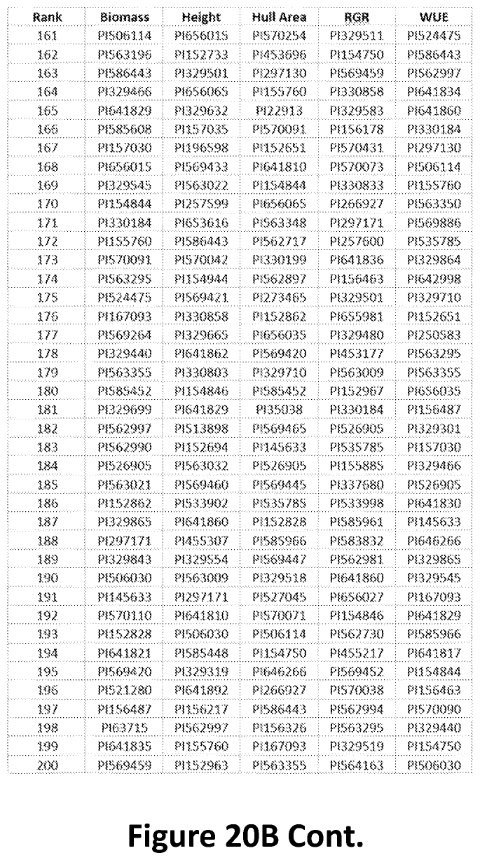

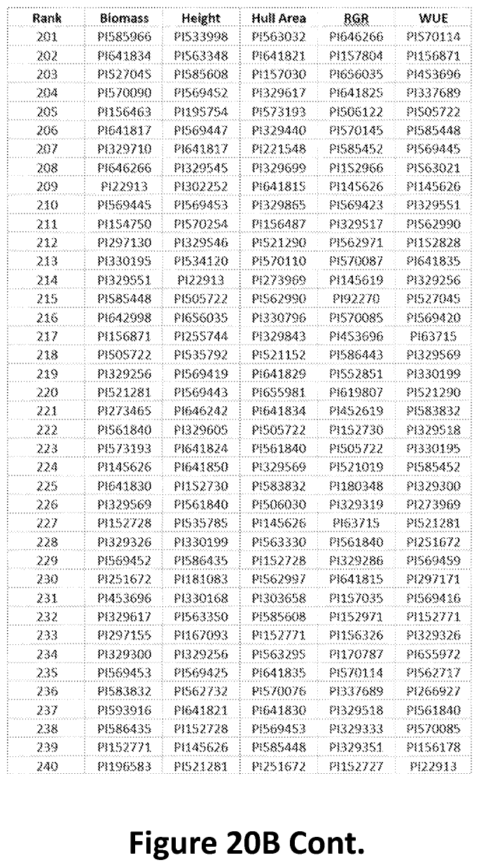

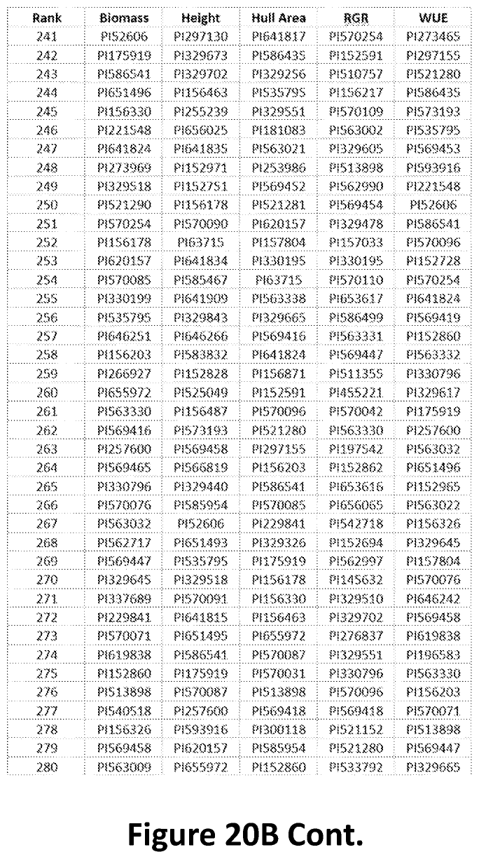

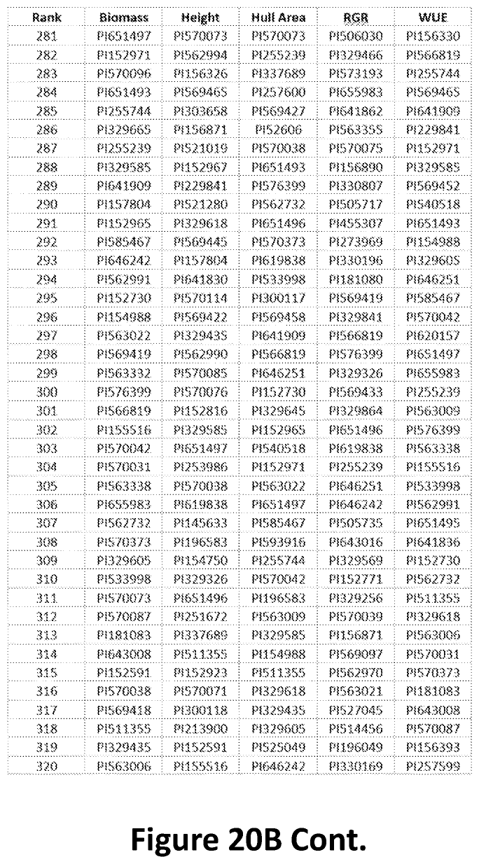

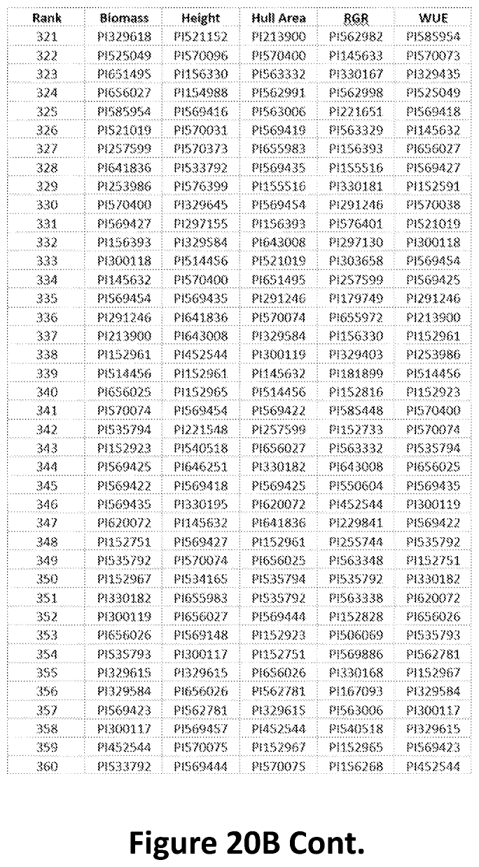

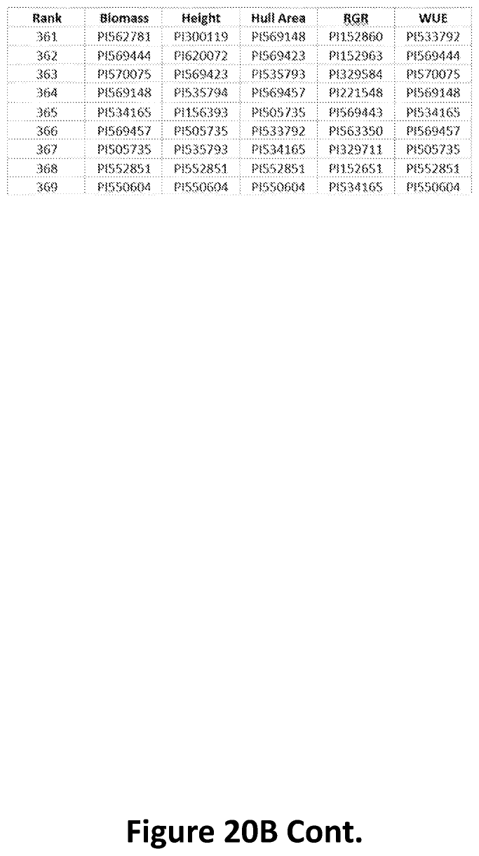

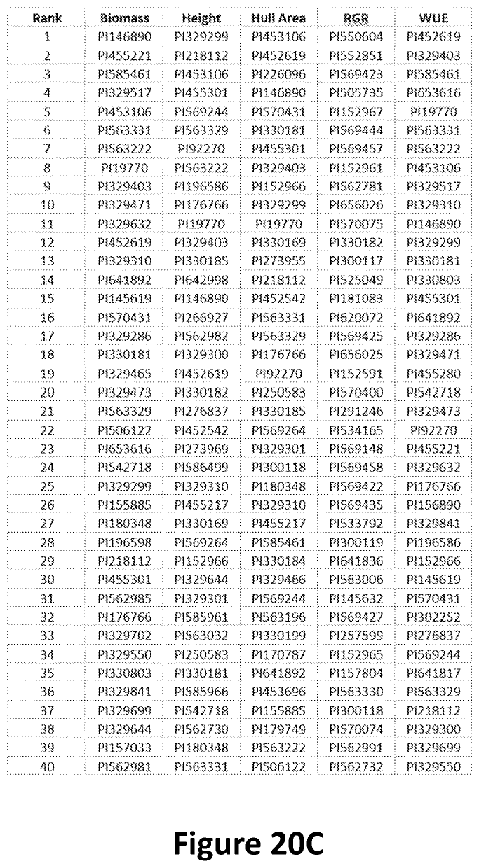

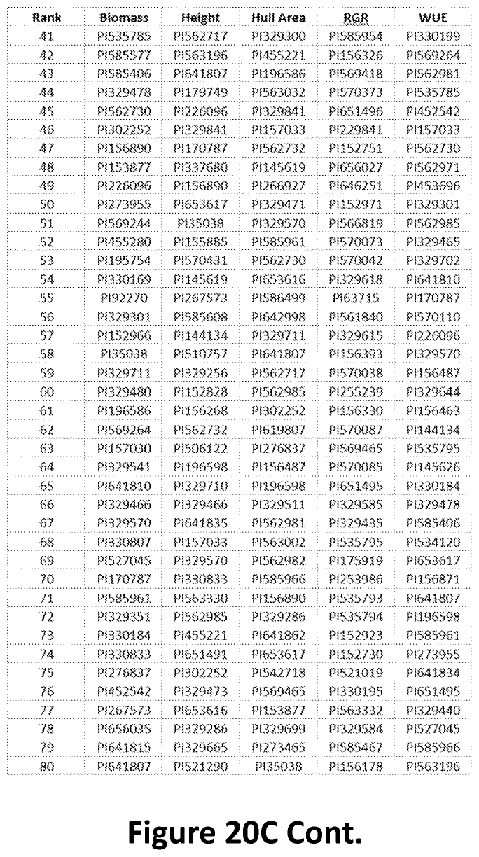

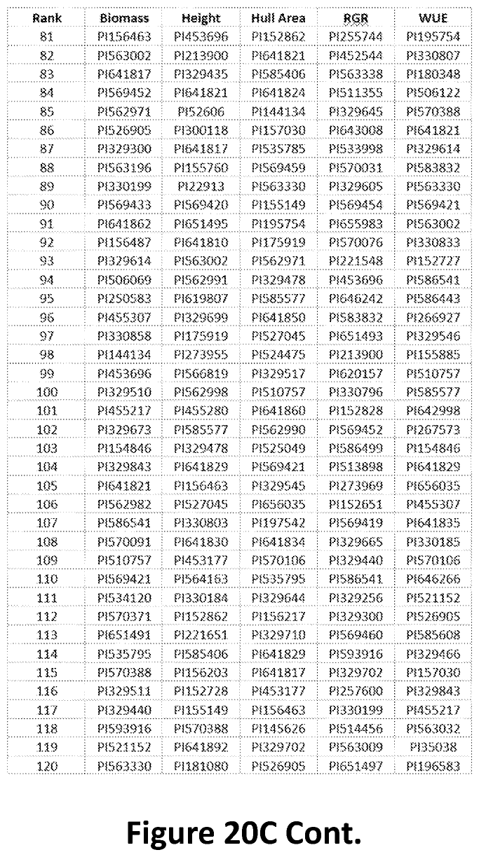

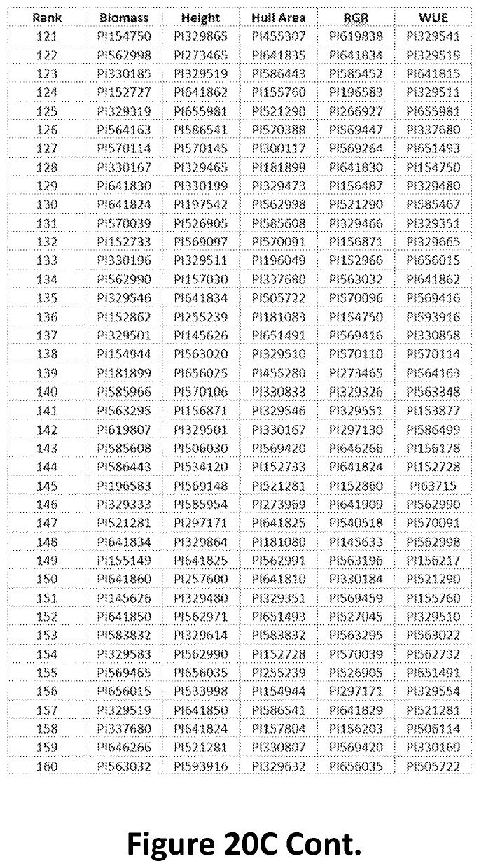

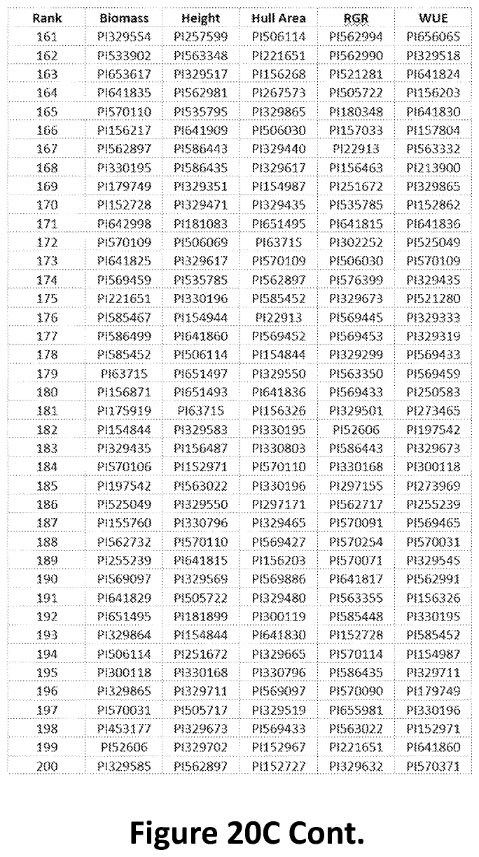

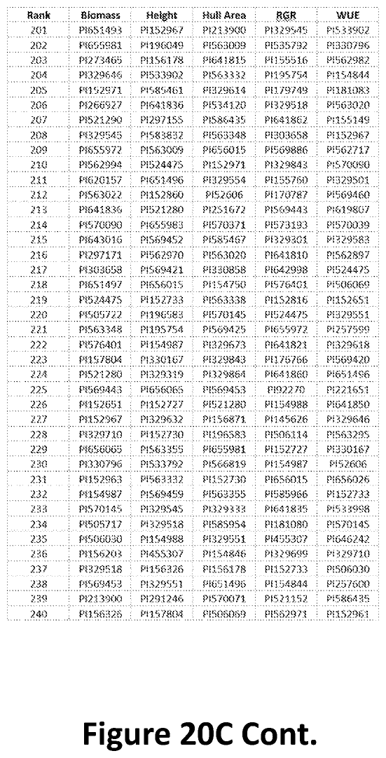

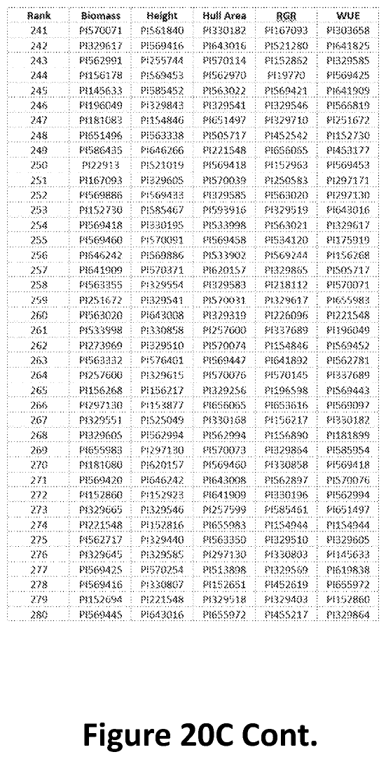

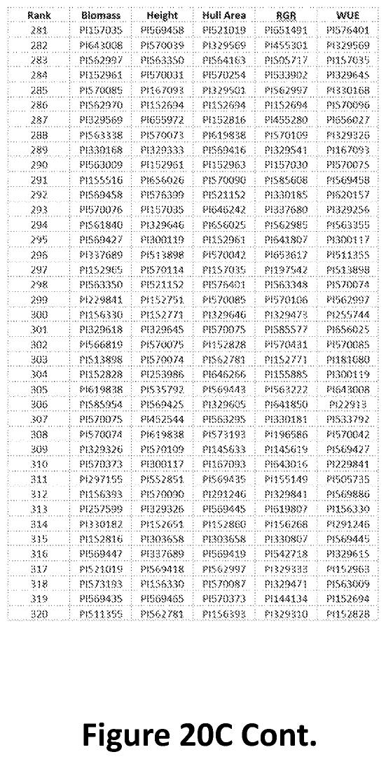

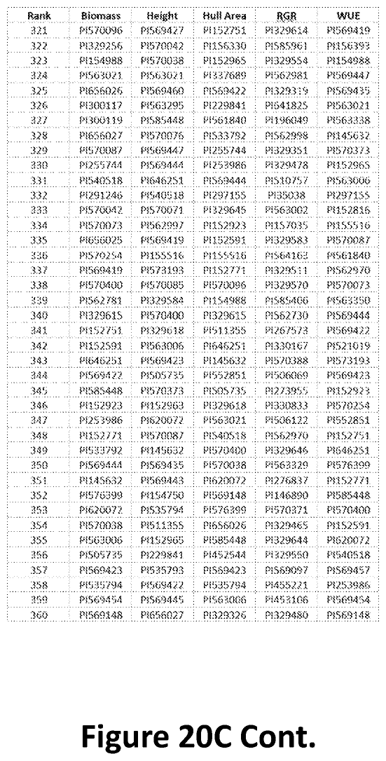

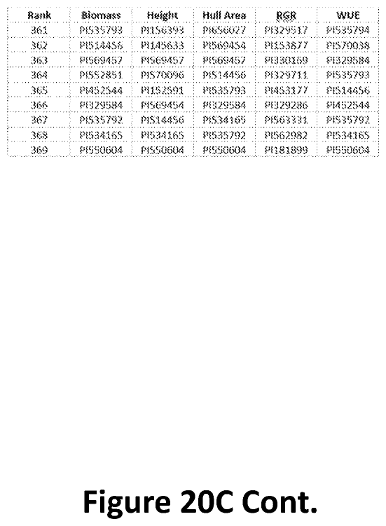

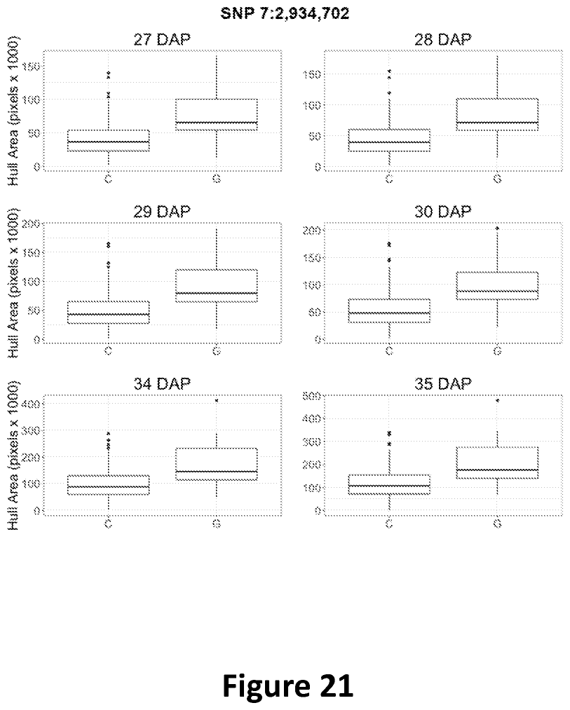

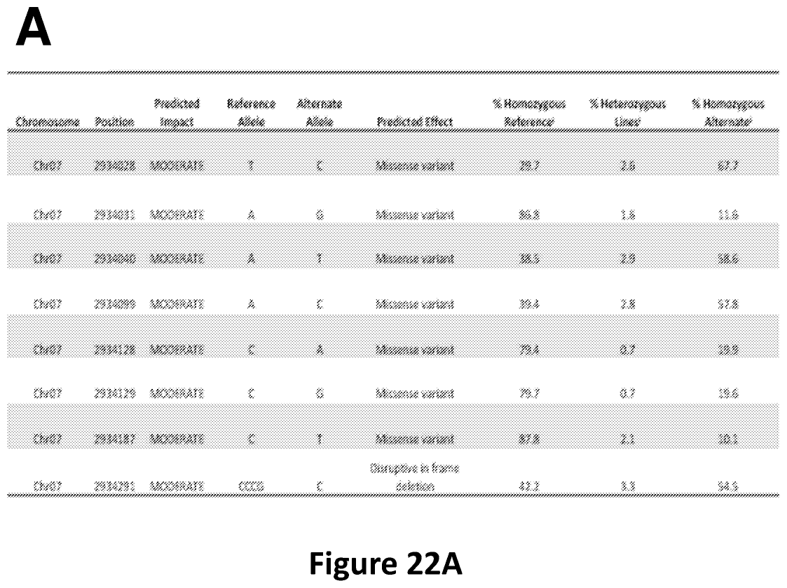

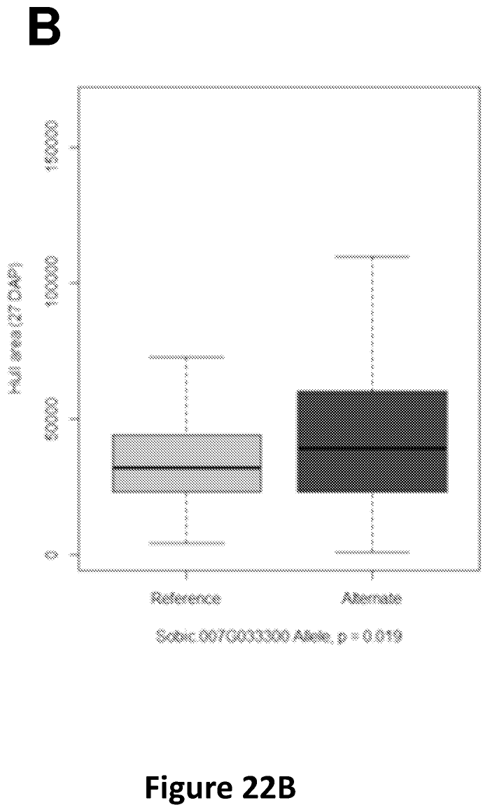

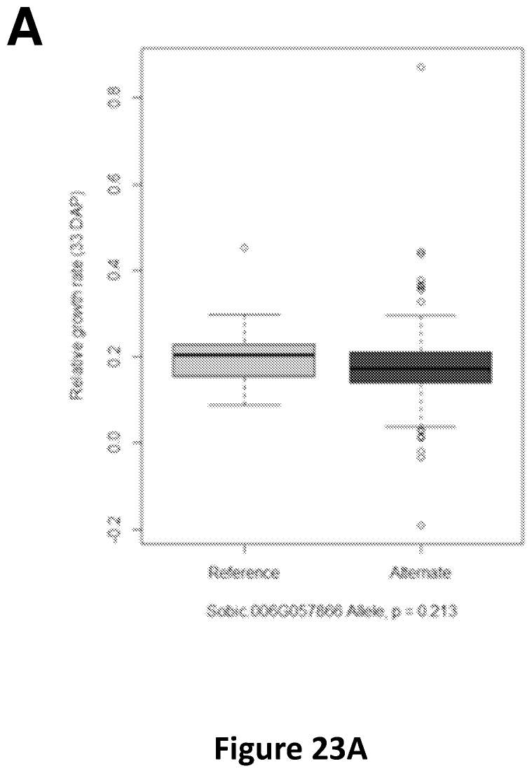

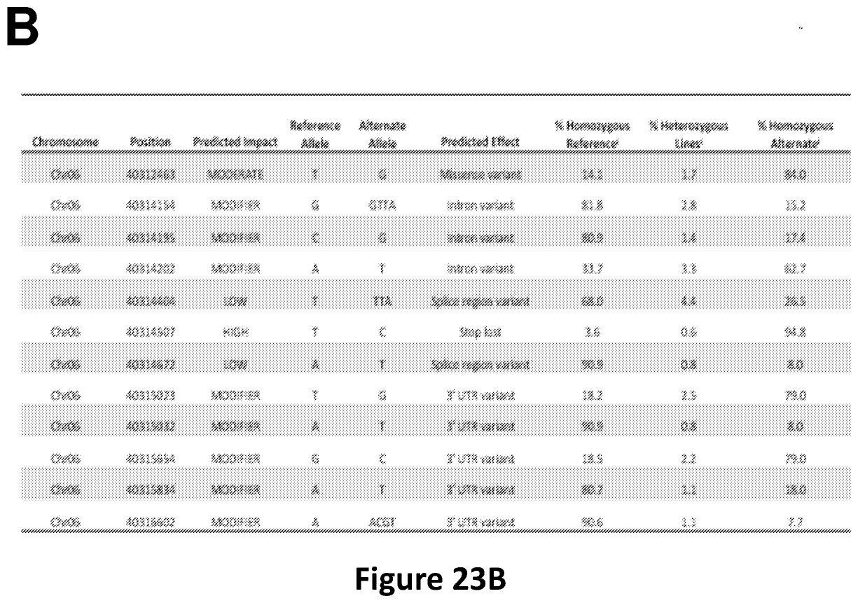

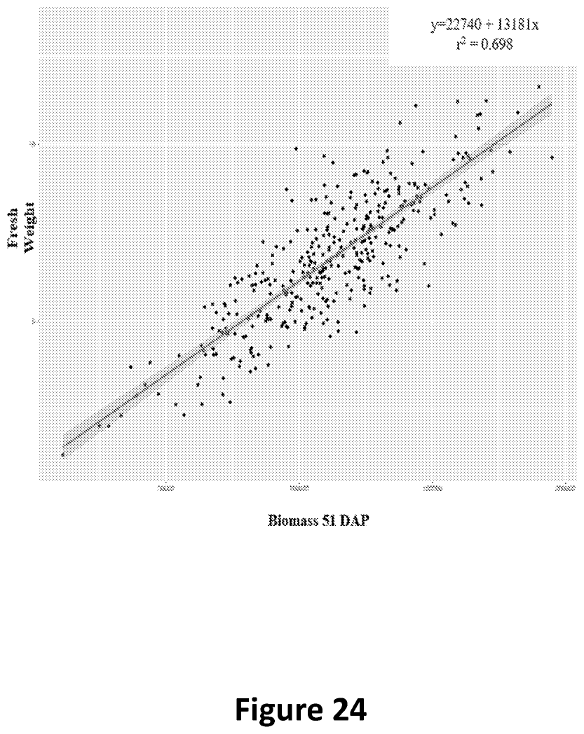

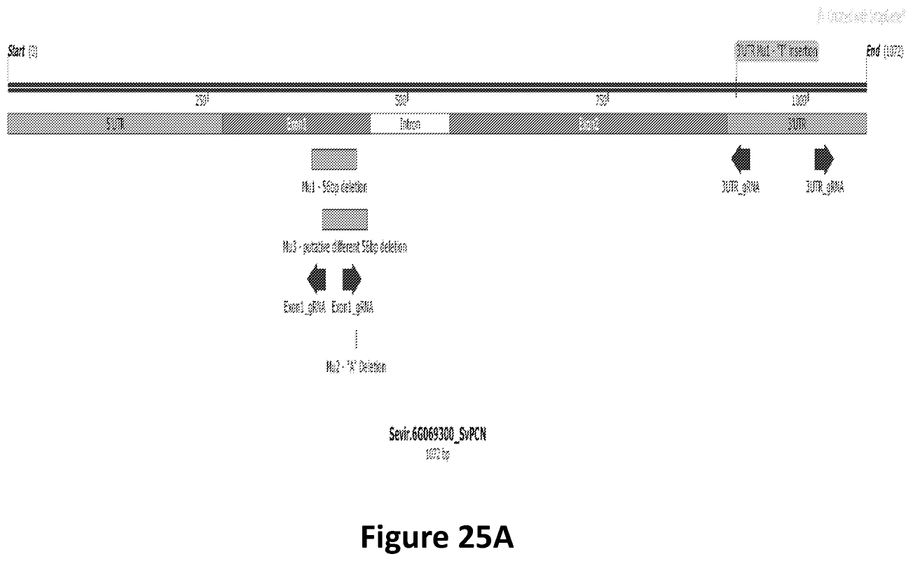

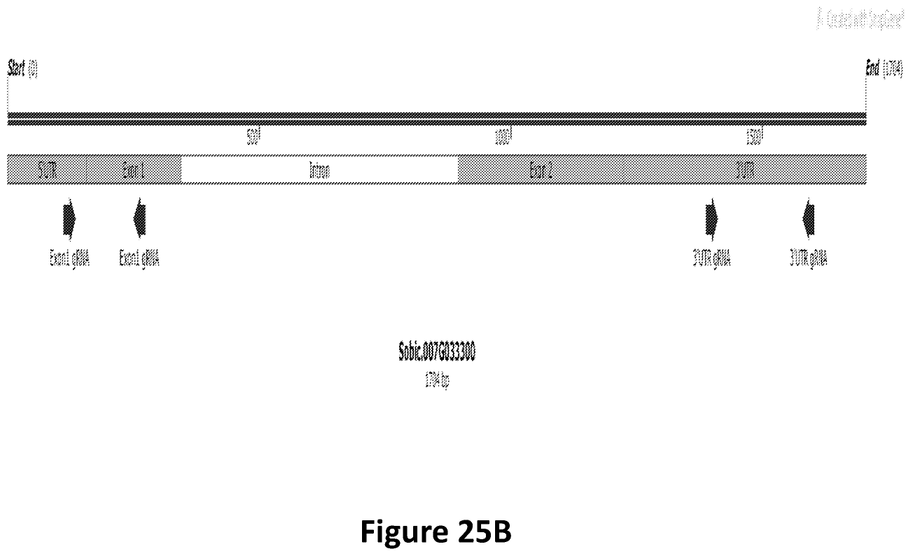

The location of two deletions (Mu1 and Mu2) in plastocyanin (Sevir.6G069300) Exon 1 are shown in FIG. 13. Mu1 is a 56 bp deletion between the two gRNA target sites resulting in a frameshift mutation. Mu2 is a single base pair deletion of an “A” at base 167 of Exon 1, which is within one of the gRNA target sites; resulting in a nonsense mutation generating a premature stop codon at base 42 of Exon 2, base 594. FIG. 14 shows the sequence underlying the Mu1 and Mu2 Exon 1 deletions. FIG. 15 shows the sequence variation for Mu3 in the 3′ UTR region of Sevir.6G069300. Mu3 is a single base insertion of “T” after base 910, 10 bases into the 3′ UTR. FIG. 25A shows the full length S. viridis plastocyanin gene (Sevir.6G069300) showing the target locations of the Exon 1 and 3′ UTR guide RNAs of the Exon 1 Mu1 56 bp deletion, the Exon 1 Mu2 “A” deletion, the Exon 1 putative Mu3 56 bp deletion, and the 3′ UTR Mu1 “T” insertion. FIG. 25B shows the full length Sorghum plastocyanin gene (Sobic.007G033300) showing the target locations of the 5′ UTR/Exon 1 and 3′ UTR guide RNAs.

Sevir.6G069300: misc_feature 1 . . . 269/label=5′UTR; exon 270 . . . 453/label=Exon1; misc_feature complement (375 . . . 397)/label=Exon1_gRNA; misc_feature 381 . . . 436/label=Mu1—56 bp deletion; misc_feature 394 . . . 449/label=Mu3—putative different 56 bp deletion; misc_feature 420 . . . 442/label=Exon1_gRNA; misc_feature 436/label=Mu2—““A”” Deletion; misc_feature 454 . . . 552/label=Intron; exon 553 . . . 899/label=Exon2; misc_feature 900 . . . 1072/label=3′UTR; misc_feature complement (905 . . . 927)/label=3UTR_gRNA; misc_feature 910\hat{ }911/label=3′UTR Mu1—““T”” insertion; misc_feature 1010 . . . 1032/label=3UTR_gRNA.

Sobic.007G033300: misc_feature 1 . . . 157/label=5′UTR; misc_feature 113 . . . 135/label=Exon1 gRNA; exon 158 . . . 344/label=Exon 1; misc_feature complement (251 . . . 273)/label=Exon1 gRNA; misc_feature 345 . . . 895/label=Intron; exon 896 . . . 1224/label=Exon 2; misc_feature 1225 . . . 1704/label=3′UTR; misc_feature 1389 . . . 1411/label=3′UTR gRNA; misc_feature complement (1581 . . . 1603)/label=3′UTR gRNA.

S. viridis

Plants were grown in growth chambers for chilling stress (15° C.) or control (30° C. day/20° C. night) for 12 hour days at 50% RH. In the first iteration of the experiment, the control genotype ME34 did not germinate in either temperature condition. Germination rates were poor for the mutant alleles as well, at 2/12 in the warm condition and 4/12 (Mu1) or 3/12 (Mu2) in the chilling stress condition. Growth rate and plant height are being measured semi-weekly on the plants in both temperatures. Total biomass of the plant will be measured on a fresh weight and dry weight basis at maturity. The experiment will be repeated to attempt to get better germination rates. It will also be repeated with the 3′ UTR allele to compare to wild-type growth. Plants will be characterized for gene expression differences to confirm phenotypic differences are due to on-target changes in the plastocyanin gene targeted.

Sorghum

Gene-edited sorghum plants will be characterized for changes in biomass and growth rate under chilling stress similar to the protocol for S. viridis. Plants will be characterized for gene expression differences to confirm phenotypic differences are due to on-target changes in the plastocyanin gene targeted.

TABLE 3
Sequences.

| SEQ ID NO: | Description |
|---|---|
| 1 | Sorghum plastocyanin gene Sobic.007G033300 |
| 2 | S. viridis gene Sevir.6G069300 |
| 3 | Wild-type S. viridis Sevir.6G069300 gene Exon 1 |
| 4 | Wild-type S. viridis Sevir.6G069300 gene 3′ UTR |
| 5 | Wild-type Sorghum plastocyanin gene 5′ UTR and Exon 1 |
| 6 | Wild-type Sorghum plastocyanin gene 3′ UTR |
| 7 | S. viridis Exon 1 Mu1 variant encoded polypeptide |
| 8 | S. viridis Exon 1 Mu1 variant polynucleotide |
| 9 | S. viridis Exon 1 Mu2 variant encoded polypeptide |
| 10 | S. viridis Exon 1 Mu2 variant polynucleotide |
| 11 | S. viridis 3′ UTR Mu1 variant polynucleotide |
| 12 | Wild-type Sorghum plastocyanin protein |
| 21 | Wild-type Sorghum plastocyanin gene 5′ UTR |
| 22 | Wild-type Sorghum plastocyanin gene Exon 1 |

TABLE 4
Table of 72 candidate genes.

| | | | | | Distance to | |
|---|---|---|---|---|---|---|
| — | Candidate Gene | ID | Trait | Basepair | SNP (kbp) | Alleles |
| 1 | Sobic.001G012300 | Sb01g001200 | Biomass | 1,153,543 | 15 | T/G |
| | | | 8 DAP | | | |
| 2 | Sobic.001G104100 | Sb01g009300 | Hull Area | 7,961,525 | 100 | A/G |
| | | | 53-56 | | | |
| | | | DAP | | | |
| 3 | Sobic.001G104500 | Sb01g009330 | Hull Area | 7,961,525 | 100 | A/G |
| | | | 53-56 | | | |
| | | | DAP | | | |
| 4 | Sobic.001G141800 | Sb01g012340 | RGR 25 | 11,389,602 | 0 | G/A |
| | | | DAP | | | |
| 5 | Sobic.001G141900 | | RGR 25 | 11,389,602 | 15 | G/A |
| | | | DAP | | | |
| 6 | Sobic.001G170600 | Sb01g014920 | Biomass | 14,239,780 | 15 | C/G |
| | | | 7 DAP, | | | |
| | | | WUE 7 | | | |
| | | | DAP | | | |
| 7 | Sobic.001G293100 | | RGR 31 | 57,023,021 | 15 | T/A |
| | | | DAP | | | |
| | | | RGR 31 | 57,023,021 | 15 | T/A |
| | | | DAP | | | |
| | | | RGR 31 | 57,023,021 | 15 | T/A |
| | | | DAP | | | |
| | | | RGR 31 | 57,023,021 | 15 | T/A |
| | | | DAP | | | |
| 8 | Sobic.001G323801 | Sb01g031380 | RGR 25 | 61,139,721 | 15 | C/A |
| | | | DAP | | | |
| | | | RGR 36 | | | |
| | | | DAP | | | |
| 9 | Sobic.001G323900 | Sb01g031390 | RGR 25 | 61,139,721 | 15 | C/A |
| | | | DAP | | | |
| | | | RGR 36 | | | |
| | | | DAP | | | |
| 10 | Sobic.001G324100 | | RGR 25 | 61,139,721 | 15 | C/A |
| | | | DAP | | | |
| | | | RGR 36 | | | |
| | | | DAP | | | |
| 11 | Sobic.001G427500 | Sb01g040160 | WUE 8 | 70,671,169 | 0 | C/A |
| | | | DAP | | | |
| 12 | Sobic.001G500800 | Sb01g046790 | RGR 25 | 77,004,071 | 5 | G/A |
| | | | DAP | | | |
| 13 | Sobic.001G500900 | Sb01g046800 | RGR 25 | 77,004,071 | 15 | G/A |
| | | | DAP | | | |
| 14 | Sobic.001G506100 | Sb01g047250 | RGR 31 | 77,439,532 | 15 | A/G |
| | | | DAP | | | |
| 15 | Sobic.002G013400 | Sb02g001390 | WUE 14- | 1,199,928 | 5 | G/A |
| | | | 19 DAP, | | | |
| | | | Biomass | | | |
| | | | 14-20 | | | |
| | | | DAP | | | |
| 16 | Sobic.002G013550 | Sb02g001393 | WUE 14- | 1,199,928 | 15 | G/A |
| | | | 19 DAP, | | | |
| | | | Biomass | | | |
| | | | 14-20 | | | |
| | | | DAP | | | |
| 17 | Sobic.002G164700 | Sb02g020840 | Hull Area | 51,251,863 | 15 | G/A |
| | | | 49-52 | | | |
| | | | DAP | | | |
| 18 | Sobic.002G225100 | Sb02g026570 | RGR 27 | 61,698,343 | 15 | G/T |
| | | | DAP | | | |
| 19 | Sobic.002G272900 | Sb02g030660 | RGR 27 | 65,622,374 | 5 | G/A |
| | | | DAP | | | |
| 20 | Sobic.002G273000 | | RGR 27 | 65,622,374 | 5 | G/A |
| | | | DAP | | | |
| 21 | Sobic.002G329000 | Sb02g035550 | RGR 43 | 69,832,371 | 15 | G/T |
| | | | DAP | | | |
| 22 | Sobic.002G329100 | Sb02g035560 | RGR 43 | 69,832,371 | 15 | G/T |
| | | | DAP | | | |
| 23 | Sobic.002G329200 | Sb02g035570 | RGR 43 | 69,832,371 | 0 | G/T |
| | | | DAP | | | |
| 24 | Sobic.002G329300 | Sb02g035580 | RGR 43 | 69,832,371 | 5 | G/T |
| | | | DAP | | | |
| 25 | Sobic.002G329400 | Sb02g035590 | RGR 43 | 69,832,371 | 15 | G/T |
| | | | DAP | | | |
| 26 | Sobic.002G357900 | Sb02g037900 | RGR 24 | 72,052,186 | 15 | T/G |
| | | | DAP | | | |
| 27 | Sobic.002G388200 | Sb02g040440 | RGR 27 | 74,210,699 | 15 | A/G |
| | | | DAP | | | |
| 28 | Sobic.002G388400 | Sb02g040450 | RGR 27 | 74,210,699 | 5 | A/G |
| | | | DAP | | | |
| 29 | Sobic.002G392300 | Sb02g040770 | Biomass | 74,514,218 | 15 | T/A |
| | | | 12 DAP | | | |
| 30 | Sobic.003G022700 | Sb03g002150 | Height 8 | 1,963,989 | 5 | C/G |
| | | | DAP | | | |
| 31 | Sobic.003G153200 | Sb03g013330 | RGR 41 | 16,372,825 | 0 | G/T |
| | | | DAP | | | |
| 32 | Sobic.003G243900 | Sb03g030030 | RGR 22 | 58,326,479 | 15 | A/T |
| | | | DAP | | | |
| 33 | Sobic.003G244100 | Sb03g030050 | RGR 22 | 58,326,479 | 5 | A/T |
| | | | DAP | | | |
| 34 | Sobic.003G427500 | Sb03g045960 | Hull Area | 73,087,058 | 15 | G/T |
| | | | 50 DAP, | (Hull | | (73,087,058) |
| | | | RGR 15 | area 50 | | A/G |
| | | | DAP | DAP) | | (73,091,310) |
| | | | | 73,091,310 | | |
| | | | | (RGR 15 | | |
| | | | | DAP) | | |
| 35 | Sobic.004G081900 | | RGR 23 | 6,702,749 | 15 | C/T |
| | | | DAP | | | |
| 36 | Sobic.004G288300 | Sb04g032390 | Hull Area | 62,989,269 | 15 | C/G |
| | | | 21-25 | | | |
| | | | DAP | | | |
| 37 | Sobic.004G288400 | Sb04g032400 | Hull Area | 62,989,269 | 15 | C/G |
| | | | 21-25 | | | |
| | | | DAP | | | |
| 38 | Sobic.005G019100 | Sb05g001630 | RGR 27 | 1,730,801 | 5 | T/C |
| | | | DAP | | | |
| 39 | Sobic.005G021300 | Sb05g001805 | RGR 23 | 1,966,419 | 5 | C/G |
| | | | DAP | | | |
| 40 | Sobic.005G055900 | Sb05g004560 | RGR 22 | 5,663,603 | 5 | G/A |
| | | | DAP | | | |
| 41 | Sobic.005G056000 | | RGR 22 | 5,663,603 | 0 | G/A |
| | | | DAP | | | |
| 42 | Sobic.005G090800 | | RGR 27 | 13,378,992 | 15 | T/C |
| | | | DAP | | | |
| 43 | Sobic.005G091000 | Sb05g007480 | RGR 27 | 13,378,992 | 15 | T/C |
| | | | DAP | | | |
| 44 | Sobic.006G002100 | Sb06g000380 | RGR 39- | 382,682 | 15 | C/A |
| | | | 40 DAP | | | |
| 45 | Sobic.006G057866 | | RGR 33 | 40,312,463 | 0 | G/T |
| | | | DAP | | | |
| 46 | Sobic.006G116600 | Sb06g019790 | RGR 36 | 48,450,391 | 15 | A/G |
| | | | DAP | | | |
| 47 | Sobic.006G204400 | Sb06g027550 | RGR 24 | 55,466,730 | 15 | T/A |
| | | | DAP | 55,467,259 | | (55,466,730) |
| | | | | | | C/T |
| | | | | | | (55,467,259) |
| 48 | Sobic.006G204500 | Sb06g027560 | RGR 24 | 55,466,730 | 0 | T/A |
| | | | DAP | 55,467259 | | (55,466,730) |
| | | | | | | C/T |
| | | | | | | (55,467,259) |
| 49 | Sobic.006G204600 | Sb06g027570 | RGR 24 | 55,466,730 | 15 | T/A |
| | | | DAP | 55,467,259 | | (55,466,730) |
| | | | | | | C/T |
| | | | | | | (55,467,259) |
| 50 | Sobic.007G033300 | Sb07g002860 | Hull Area | 2,934,702 | 0 | C/G |
| | | | 27-30, | | | |
| | | | 34-35 | | | |
| | | | DAP | | | |
| 51 | Sobic.007G033400 | Sb07g002870 | Hull Area | 2,934,702 | 5 | C/G |
| | | | 27-30, | | | |
| | | | 34-35 | | | |
| | | | DAP | | | |
| 52 | Sobic.008G006800 | Sb08g000710 | RGR 30 | 575,183 | 5 | T/G |
| | | | DAP | | | |
| 53 | Sobic.008G006900 | Sb08g000720 | RGR 30 | 575,183 | 5 | T/G |
| | | | DAP | | | |
| 54 | Sobic.008G058500 | Sb08g004930 | Height 2- | 6,142,090 | 5 | A/G |
| | | | 3 DAP, | | | |
| | | | Hull Area | | | |
| | | | 2-3 DAP | | | |
| 55 | Sobic.008G118700 | Sb08g017240 | RGR 18 | 53,112,641 | 5 | T/C |
| | | | DAP | | | |
| 56 | Sobic.008G152300 | Sb08g020140 | RGR 20 | 58,474,500 | 15 | C/T |
| | | | DAP | | | |
| 57 | Sobic.008G156600 | Sb08g020560 | RGR 45 | 58,905,676 | 0 | T/C |
| | | | DAP | | | |
| 58 | Sobic.008G156900 | Sb08g020580 | RGR 45 | 58,905,676 | 15 | T/C |
| | | | DAP | | | |
| 59 | Sobic.008G168400 | Sb08g021375 | Biomass | 60,254,418 | 15 | T/C |
| | | | 19-20 | | | |
| | | | DAP | | | |
| 60 | Sobic.008G192800 | Sb08g023280 | RGR 13 | 62,617,925 | 5 | T/C |
| | | | DAP | | | |
| 61 | Sobic.009G189501 | Sb09g024850 | RGR 41- | 54,129,591 | 15 | C/T |
| | | | 42 DAP | | | |
| 62 | Sobic.009G200700 | Sb09g025820 | Biomass | 55,067,080 | 15 | G/A |
| | | | 34-38 | | | |
| | | | DAP, | | | |
| | | | WUE 35- | | | |
| | | | 36 DAP | | | |
| 63 | Sobic.009G200800 | Sb09g025830 | Biomass | 55,067,080 | 5 | G/A |
| | | | 34-38 | | | |
| | | | DAP, | | | |
| | | | WUE 35- | | | |
| | | | 36 DAP | | | |
| 64 | Sobic.009G200900 | Sb09g025840 | Biomas | 55,067,080 | 0 | G/A |
| | | | 34-38 | | | |
| | | | DAP, | | | |
| | | | WUE 35- | | | |
| | | | 36 DAP | | | |
| 65 | Sobic.009G221800 | Sb09g027580 | Hull Area | 56,526,623 | 15 | G/T |
| | | | 45 DAP, | 56,500,182 | | (56526623) |
| | | | RGR 54- | | | T/C |
| | | | 55 DAP | | | (56500182) |
| 66 | Sobic.009G221900 | Sb09g027590 | Hull Area | 56,526,623 | 15 | G/T |
| | | | 45 DAP, | 56,500,182 | | (56526623) |
| | | | RGR 54- | | | T/C |
| | | | 55 DAP | | | (56500182) |
| 67 | Sobic.009G222100 | Sb09g027610 | Hull Area | 56,526,623 | 0 | G/T |
| | | | 45 DAP, | 56,500,182 | | (56526623) |
| | | | RGR 54- | | | T/C |
| | | | 55 DAP | | | (56500182) |
| 68 | Sobic.009G237700 | Sb09g029000 | RGR 26 | 57,601,358 | 15 | A/G |
| | | | DAP | | | |
| 69 | Sobic.010G057300 | Sb10g004950 | Hull Area | 4,476,256 | 0 | T/C |
| | | | 37 DAP | | | |
| 70 | Sobic.010G088700 | Sb10g007690 | Height 5 | 7,591,313 | 5 | A/C |
| | | | DAP | | | |
| 71 | Sobic.010G088800 | Sb10g007700 | Height 5 | 7,591,313 | 15 | A/C |
| | | | DAP | | | |
| 72 | Sobic.010G246200 | Sb10g028530 | RGR 21 | 58,585,447 | 5 | C/A |
| | | | DAP | 58,585,570 | | (58585447) |
| | | | | | | C/T |
| | | | | | | (58585570) |

S10 cont.

| — | Description | Homolog | Homolog Description |
|---|---|---|---|
| 1 | Similar to Cytochrome P450 | AT4G39950 (70.3%) | CYP79B2, a gene involved in the Indolic |
| | | | glucosinolate pathway, is downregulated when |
| | | | plants are grown for three generations in the cold |
| | | | (9° C.) (38). |
| | | | A cold-affected, auxin biosynthesis gene (39) . |
| | | | Decreased expression in ptl-D plants. ptl-D was |
| | | | defective in auxin action, and the alteration of |
| | | | auxin distribution corresponded to the curly leaf |
| | | | phenotype (40). |
| | | AT2G22330 (69.4%) | CYP79B3, a gene involved in the Indolic |
| | | | glucosinolate pathway, is downregulated when |
| | | | plants are grown for three generations in the cold |
| | | | (9° C.) (38). |
| | | AT1G16410 (60.8%) | CYP79F1 upregulated when grown for three |
| | | | generations in the cold (9° C.) (38). |
| | | AT5G07990 (47.7%) | Putative cold incuded gene (41). |
| | | | Sugarcane homolog had increased expression |
| | | | when leaves cold-girdled for 56 hours (42). |
| | | GRMZM2G140817 (45.7%) | Encodes coumarate 3-hydroxylase (C3H) and |
| | | | plays a vital role in the lignin biosynthesis |
| | | | pathway (43). |
| 2 | Similar to Putative ACT | AT4G22780 (48.6%) | Induced by myb4. OsMyb4 has been isolated as a |
| | domain containing protein | | cold inducible gene (44). |
| 3 | Growth regulating factor 1 | AT2G22840 (42.3%) | Plants that overexpressied AtGRF1 developed |
| | related | | larger leaves with longer petioles (45). |
| | | AT4G37740 (43.4%) | Plants that overexpressied AtGRF2 developed |
| | | | larger leaves with longer petioles (45). |
| | | | Expression of GRF2 increased in gra-D mutants. |
| | | | Grandifolia-D (gra-D) mutants of Arabidopsis |
| | | | thaliana were characterized that developed huge |
| | | | leaves with two to three times more cells than the |
| | | | wild-type (46). |
| 4 | Similar to Putative | AT4G23180 (59.3%) | Cold regulated gene in Thlaspi arvense (47). |
| | serine/threonine kinase | | |
| 5 | Protein kinase PCTAIRE and | AT5G67360 (39.5%) | Peach homolog is cold induced in mesocarp |
| | related kinases // Protein kinase | | tissue (48). |
| | PITSLRE and related | | |
| | kinasesLinksB M | | |
| 6 | F Box domain | Os04g02280 (58.4%) | Differentially expressed in cold tolerant and cold |
| | | | sensitive lines (49). |
| 7 | Peroxidase/Lactoperoxidase | AT5G17820 (55.4%) | Increased expression after short term (3 hours) |
| | | | and long term (48 hours) chilling stress (50). |
| | Peroxidase/Lactoperoxidase | AT4G11290 (57.2%) | Increased expression in brassinosteroid- |
| | | | insensitive mutants, bri1-9, which showed |
| | | | increased cold tolerance compared with both |
| | | | wild-type and BRI1-overexpressing transgenic |
| | | | plants (51). |
| | | | Increased expression in line overexpressing |
| | | | MeDREB1D(Y-3). MeDREB1D(Y-3) |
| | | | overexpression resulted in stronger tolerance to |
| | | | drought and cold stresses (52). |
| | Peroxidase/Lactoperoxidase | AT3G21770 (53.5%) | Increased expression in brassinosteroid- |
| | | | insensitive mutants, bri1-9, which showed |
| | | | increased cold tolerance compared with both |
| | | | wild-type and BRI1-overexpressing transgenic |
| | | | plants (51). |
| | Peroxidase/Lactoperoxidase | AT5G64120 (51.7%) | Increased expression in line overexpressing |
| | | | MeDREB1D. MeDREB1D overexpression |
| | | | resulted in stronger tolerance to drought and cold |
| | | | stresses (52). |
| | | | AtPRX71might contribute to the growth |
| | | | reduction observed in fungal polygalacturonase |
| | | | expressing plants by promoting ROS production |
| | | | (53). |
| 8 | Invertase/pectin methylesterase | AT5G62360 (38.9%) | Cold induced Invertase/pectin methylesterase |
| | inhibitor family | | inhibitor (41). |
| | | | Peach homolog preferentially expressed in cold |
| | | | stored peach fruits (54). |
| | | | Cold induced gene with increased expression |
| | | | after exposure to 5° C. for 6 hours compared to |
| | | | ambient temperature of 20° C. (55). |
| | | AT5G62350 (38.1%) | Upregulated by DREB1A overexpression and |
| | | | cold stress (56). |
| | | | Cold induced invertase/pectin methylesterase |
| | | | inhibitor family protein (41). |
| | | AT4G25260 (37.65) | Pectinesterase upregulated during cold |
| | | | deacclimation (57). |
| | | AT4G12390 (36.7%) | Cold repressed methylesterase inhibitor family |
| | | | protein PME1 (41). |
| 9 | Similar to Putative | GRMZM2G083000 (38.5%) | GRMZM2G083000 lacks the F-box domain and |
| | uncharacterized protein | | thus is a potential candidate for interaction with |
| | | | ZmFBX92. The F-box protein FBX92 has been |
| | | | identified as a repressor of leaf growth in |
| | | | Arabidopsis. Overexpression of AtFBX92 resulted |
| | | | in plants with smaller leaves than the wild type, |
| | | | whereas plants with reduced levels of AtFBX92 |
| | | | showed, in contrast, increased leaf growth by |
| | | | stimulating cell proliferation (58). |
| 10 | oxidoreductase | AT1G17020 (56.5%) | In the elongating maize internode, transciption of |
| | | | the maize homolog of AT1G17020, carboxylate |
| | | | oxidase (Q75IP9), a lignin metabolism gene, is |
| | | | highly correlated with secondary cell wall |
| | | | cellulase synthase ZmCesA genes (59). |
| | | AT5G05600 (47.6%) | Flavonol synthase gene upregulated in freezing |
| | | | tolerant plants (60). |
| | | AT5G54000 (51.3%) | Oxidoreductase gene with increased expression |
| | | | under cold stress in Thlaspi arvense (47). |
| 11 | Similar to Putative | AT5G41330 (42.7%) | Potato homolog upregulated in plants |
| | uncharacterized protein | | overexpressing CaWRKY1. The overexpression of |
| | | | CaWRKY1 in potato plants increased tolerance |
| | | | to drought stress without noticeably affecting |
| | | | other agricultural important factors, such as |
| | | | plant height, leaf size, and tuber formation (61). |
| | | | Differentially expressed in 35S:ABF3 plants that |
| | | | may contribute to drought-tolerance (62). |
| 12 | Similar to Os03g0152400 | AT1G51680 (61.7%) | Increased expression under cold treatment (3° C.) |
| | protein | | (63). |
| | | AT3G48990 (48.5%) | AMP-dependent synthetase enriched in Korean |
| | | | rapeseed cultivars during early times of cold |
| | | | stress (64). |
| 13 | similar to Class III peroxidase | AT5G17820 (42.8%) | Increased expression after short term (3 hours) |
| | 36 precursor | | and long term (48 hours) chilling stress (50). |
| | | AT5G64120 (41.6%) | Increased expression in line overexpressing |
| | | | MeDREB1D. MeDREB1D overexpression |
| | | | resulted in stronger tolerance to drought and cold |
| | | | stresses (52). |
| | | | AtPRX71 might contribute to the growth |
| | | | reduction observed in fungal poly galacturonase |
| | | | expressing plants by promoting ROS production |
| | | | (53). |
| | | | Increased expression in bri mutant and BRI1 |
| | | | overexpressing plants compared with those of |
| | | | wild type plants. Mutant alleles that are |
| | | | defective in BRI1 display severely growth- |
| | | | inhibited dwarf phenotypes, whereas BRI1- |
| | | | overexpressing transgenic plants appear as |
| | | | constitutive BR-treated phenotypes with longer |
| | | | and narrower growth in their leaves and petioles |
| | | | (51). |
| | | AT5G39580 (40.7%) | Expression induced after drought treatment in |
| | | | lines overexpressing cold shock domain protein |
| | | | (65). |
| | | | Increased expression in bri mutant and BRI1 |
| | | | overexpressing plants compared with those of |
| | | | wild type plants. mutant alleles that are |
| | | | defective in BRI1 display severely growth- |
| | | | inhibited dwarf phenotypes, whereas BRI1- |
| | | | overexpressing transgenic plants appear as |
| | | | constitutive BR-treated phenotypes with longer |
| | | | and narrower growth in their leaves and petioles |
| | | | (51). |
| | | | Increased expression in line overexpressing |
| | | | MeDREB1D. MeDREB1D overexpression |
| | | | resulted in stronger tolerance to drought and cold |
| | | | stresses (52). |
| | | AT1G14550 (43.9%) | Increased expression in line overexpressing |
| | | | MeDREB1D. MeDREB1D overexpression |
| | | | resulted in stronger tolerance to drought and cold |
| | | | stresses (52). |
| | | AT3G50990 (40.3%) | Differentially expressed in response to cold |
| | | | temperature (66). |
| | | AT1G05260 (41.2%) | Increased expression in brassinosteroid- |
| | | | insensitive mutants, bri1-9, which showed |
| | | | increased cold tolerance compared with both |
| | | | wild type and BRI1-overexpressing transgenic |
| | | | plants (51). |
| | | AT1G05240 (41.2%) | Increased expression in brassinosteroid- |
| | | | insensitive mutants, bri1-9, which showed |
| | | | increased cold tolerance compared with both |
| | | | wild type and BRI1-overexpressing transgenic |
| | | | plants (51). |
| | | AT5G05340 (39.6%) | Increased expression in line overexpressing |
| | | | MeDREB1D. MeDREB1D overexpression |
| | | | resulted in stronger tolerance to drought and cold |
| | | | stresses (52). |
| | | | Up-regulated in response to cold stress (67). |
| 14 | Similar to Os03g0145000 | AT5G65700 (57.6%) | BAM1 is preferentially expressed in the root tip, |
| | protein | | including the quiescent center and its |
| | | | surrounding stem cells. Three receptors, BAM1, |
| | | | RPK2 and CLV2-CRN/SOL2, are involved in |
| | | | analogous signaling processes to restrict cell |
| | | | proliferation in the RAM (68). |
| | | AT4G20270 (56.3%) | C. bungeana homolog downregulated by chilling |
| | | | stress (69). |
| | | AT5G48940 (57.8%) | Expression of RGI2, which is driven by its own |
| | | | promoter, is abundantly expressed in the root |
| | | | meristem zone, and can greatly reverse the short |
| | | | root and small meristem phenotype (70). |
| | | | RGFR2 interacts with RGF peptides in |
| | | | Arabidopsis, is involved in the perception of root |
| | | | meristem growth factor (71). |
| | | AT3G24240 (53.4%) | RGFR1, a leucine-rich repeat RK that directly |
| | | | interacts with RGF peptides in Arabidopsis, is |
| | | | involved in the perception of root meristem |
| | | | growth factor (71). |
| | | | RGI1 may be detrimental to normal root growth |
| | | | and development (70). |
| | | Os04g04330 (55.1%) | ORF4 has been identified as the probable |
| | | | candidate gene for CTB4a. The expression of |
| | | | CTB4a was highly induced in panicles and leaves |
| | | | of a cold tolerant line by exposure to cold stress |
| | | | (72). |
| 15 | Weakly similar to Fusarium | Os04g30660 (21.3%) | Differentially expressed in lowland and upland |
| | resistance protein I2C-5-like | | rice under drought conditions (73). |
| 16 | ubiquitin C (UBC) | AT2G36170 (42.3%) | A candidate protein for interacting with |
| | | | Arabidopsis COLD SHOCK DOMAIN |
| | | | PROTEIN 3 (AtCSP3) by yeast 2 hybrid (74). |
| | | Os02g06640 (66.7%) | Upregulated in leaves under drought stress (75). |
| | | Os09g31031 (41.4%) | Showed upregulation in panicle tissue under |
| | | | water-deficit stress (76). |
| 17 | Similar to Putative WIP1 | AT3G45260 (21.4%) | Cold regulated transcription factor (77). |
| | protein | | |
| 18 | Similar to Putative | AT4G34000 (53.3%) | Poplar homolog involved in response to cold |
| | uncharacterized protein | | stress that is differentially expressed in active and |
| | | | dormant cambium from poplar (78). |
| | | AT1G49720 (51.4%) | ABA-responsive element binding factor that is |
| | | | cold inducible (39). |
| | | | Transcription factor with a highly preferable |
| | | | ICE1-binding cis-element (79). |
| | | | Poplar homolog involved in response to cold |
| | | | stress that is differentially expressed in active and |
| | | | dormant cambium from poplar (78). |
| | | | Cold induced gene (80). |
| | | Os06g10880 (58.5%) | Upregulated under cold stress (81). |
| | | GRMZM2G033413 (24.3%) | Upregulated under cold stress (82). |
| 19 | Similar to Homeodomain | AT2G46680 (34.6%) | Increased expression in esk1 mutants. The |
| | leucine zipper protein | | eskimo1 (esk1) mutation resulted in a 5.5° C. |
| | | | improvement in freezing tolerance in the absence |
| | | | of cold acclimation (83). |
| | | | Highly induced after cold stress (84). |
| | | | Highly over-expressed in esk1 mutants. esk1 |
| | | | mutants were more tolerant to freezing, after |
| | | | acclimation, than wild type (85). |
| | | AT3G61890 (35.0%) | Transcription factor with a highly preferable |
| | | | ICE1-binding cis-element and CBF3-binding |
| | | | ciselement in the promoter. Cold acclimation |
| | | | induces expression of the C-repeat binding |
| | | | transcription factors (CBF), which in turn |
| | | | activates downstream genes that confer chilling |
| | | | and freezing tolerance to plants. CBF is induced |
| | | | by ICE1 (inducer of CBF expression 1) (79). |
| | | | Cold induced gene (84). |
| | | | Increased expression in esk1 mutants. The |
| | | | eskimo1 (esk1) mutation resulted in a 5.5° C. |
| | | | improvement in freezing tolerance in the absence |
| | | | of cold acclimation (83). |
| | | AT1G69780 (26.7%) | ATHB13 is induced in S. lycopersicum after cold |
| | | | treatment for 12 hours (86). |
| | | AT5G53980 (32.1%) | Transcription factor up-regulated during cold |
| | | | deacclimation (57). |
| | | Os09g21180 (28.4%) | Increased expression under cold stress with |
| | | | overexpression of SNAC2. SNAC is thought to |
| | | | be induced under cold stress (87). |
| | | GRMZM2G097349 (30.9%) | Increased expression in IN13 (elongating |
| | | | internode) compared to IN9 (lignifying |
| | | | internode) (88). |
| 20 | Leucine rich repeat proteins | AT2G25490 (44.2%) | EBF1 expression induced at 3 hours of cold |
| | | | treatment and then decreased to the basal level |
| | | | after 6 hours of cold treatment (89). |
| | | | EIN3 levels are controlled by EBF1 and EBF2 F- |
| | | | box proteins. EIN3 overexpression in |
| | | | Arabidopsis leads to slower growth and the EIN3 |
| | | | transcription factor is required for long-term |
| | | | growth inhibition response to ethylene in |
| | | | Arabidopsis (90). |
| | | AT5G25350 (49.0%) | EBF2 is paralogous to EBF1, and overexpression |
| | | | of EBF1 results in plants insensitive to |
| | | | ethylene. Ethylene is a plant hormone involved in |
| | | | leaf expansion (91). |
| | | | EBF1 and -2 work coordinately in SCF |
| | | | complexes to repress ethylene action and levels |
| | | | of EIN3 are increased in mutants affecting either |
| | | | EBF1 or -2. The SCFEBF1/EBF2-dependent |
| | | | ubiquitination and subsequent removal of EIN3 |
| | | | is critical not only for proper ethylene signaling |
| | | | but also for growth in plants (92). |
| | | | EBF2 regulates ethylene signaling via |
| | | | degradation of the transcription factor |
| | | | ETHYLENE INSENSITIVE3 (EIN3) (93). |
| | | Os02g10700 (42.7%) | Activation of OsEIN2 leads to enhanced |
| | | | degradation of OsEIL1 and OsEIL2 mediated by |
| | | | the OsFBL7 (Os02g10700) and OsFBL30 F-box |
| | | | proteins. This leads to lower levels of OsEIL1 and |
| | | | OsEIL2, resulting in higher levels of GA and |
| | | | enhanced growth. FBL7 (Os02g10700) may have |
| | | | a role in the slow onset of growth stimulation |
| | | | caused by the application of ethylene (90). |
| 21 | Similar to Putative | AT4G23180 (55%) | Up-regulated serine/threonine kinase-related |
| | uncharacterized protein | | protein under cold treatment in Thlaspi arvense |
| | | | (47). |
| 22 | Similar to Putative | AT4G23180 (56.3%) | Up-regulated serine/threonine kinase-related |
| | uncharacterized protein | | protein under cold treatment in Thlaspi arvense |
| | | | (47). |
| 23 | Similar to Os07g0542900 | AT1G64640 (35.3%) | Up-regulated under cold treatment in Thlaspi |
| | protein | | arvense (47). |
| 24 | Similar to Putative | AT4G14410 (46.1%) | Down regulated transcription factor under cold |
| | uncharacterized protein | | temperatures (77). |
| | | GRMZM2G001930 (20.5%) | Down-regulated under cold stress (82). |
| 25 | Similar to Beta-amylase | AT4G17090 (48.5%) | During cold acclimation there was a significant |
| | | | increase in AtBMY8 expression. The cold |
| | | | enhancement of AtBMY8 expression is sustained |
| | | | after development of new leaves at 5° C. (94). |
| | | | Differentially expressed under cold stress (63). |
| | | Os07g35880 (75%) | Downregulated BMY gene in OsMYB30 |
| | | | overexpressed plants, and up-regulated in the |
| | | | osmyb30 mutant. Overexpression of OsMYB30 |
| | | | in rice resulted in increased cold sensitivity, |
| | | | while the osmyb30 knockout mutant showed |
| | | | increased cold tolerance (95). |
| 26 | Similar to Putative | AT1G70630 (22.9%) | Mutant REDUCED ARABINOSE YARIVI |
| | uncharacterized protein | | (RAY1) lines lacking At1g70630 resulted in |
| | OJ1047_C01.5 | | shorter primary root length and smaller rosette |
| | | | diameter compared to the wild-type (96). |
| 27 | Predicted protein | Os03g25620 (60.8%) | Gene identified as encoding a low temperature |
| | | | viability protein (97). |
| 28 | Similar to Ripening-related | AT5G02230 (72.3%) | Cold responsive gene (48). |
| | protein-like | | |
| 29 | Similar to Putative | AT4G11290 (55.4%) | Increased expression in brassinosteroid- |
| | uncharacterized protein | | insensitive mutants, bri1-9, which showed |
| | | | increased cold tolerance compared with both |
| | | | wild type and BRI1-overexpressing transgenic |
| | | | plants (51). |
| | | | Increased expression in line overexpressing |
| | | | MeDREB1D(Y-3). MeDREB1D(Y-3) |
| | | | overexpression resulted in stronger tolerance to |
| | | | drought and cold stresses (52). |
| | | Os03g25340 (70.9%) | Differentially expressed in SNAC2 |
| | | | overexpressed rice plants. SNAC2 confers cold |
| | | | tolerance (87). |
| | | GRMZM2G129935 (61.7%) | Present only in high biomass yielding lines in |
| | | | study comparing high biomass lines to low |
| | | | biomass lines (98). |
| 30 | Similar to GA 2-oxidase 5 | AT1G78440 (57.6%) | Brassica napus homolog identified as a candidate |
| | | | gene for plant height (99). |
| | | | Increased expression in ADC2 overexpressed |
| | | | line that showed drwarfism (100). |
| | | AT1G30040 (57.9%) | Transgenic aspen plants tissue-specifically |
| | | | expressing AtGA2ox2 (AT1G30040) exhibited a |
| | | | lower growth rate than the wild-type plants, |
| | | | resulting in reduced shoot elongation (101). |
| | | | GA2-oxidase (At1g30040) was down-regulated |
| | | | under chilling conditions, so that flux through the |
| | | | GA biosynthetic pathway to be increased (102). |
| | | Os01g55240 (63.8%) | Downregulated in rice mutant high-tillering |
| | | | dwarf 3 (htd3) plants exhibiting more tiller |
| | | | number and shorter culm length than the wild- |
| | | | type (103). |
| | | | Upregulated in cold tolerant line compared to |
| | | | cold sensitive line (104). |
| | | GRMZM2G427618 (65.9%) | Identified as a cold responsive differentially |
| | | | expressed gene. GRMZM2G427618 exhibited |
| | | | stready upregulation over 24 hours of cold |
| | | | treatment (105). |
| 31 | Similar to Putative | AT1G48100 (61%) | At1g48100 identified as functioning in the |
| | polygalacturonase PG2 | | expansion of multiple growing tissues and named |
| | | | POLYGALACTURONASE INVOLVED IN |
| | | | EXPANSION3 (PGX3) (106). |
| | | | Transcript level of poly galacturonase decreased |
| | | | in atnup160-1 mutant plants. atnup160-1 plants |
| | | | had altered cold-responsive gene expression and |
| | | | sensitivity to chilling stress and were defective in |
| | | | acquired freezing tolerance (107). |
| | | AT1G60590 (47.7%) | Polygalacturonase upregulated by chilling (102). |
| | | AT3G26610 (47.1%) | POLYGALACTURONASE INVOLVED IN |
| | | | EXPANSION1 (PGX1) is involved in cell |
| | | | elongation and is expressed in expanding tissues |
| | | | throughout development, including seedlings, |
| | | | roots, leaves, and flowers (108). |
| 32 | Similar to Acid phosphatase- | AT1G14290 (66.3%) | AT1G14290 (SBH2) encodes an LCB C-4 |
| | like | | hydroxylase gene. sbh1-1 sbh2-1 double mutants |
| | | | displayed severe alterations in growth (109). |
| 33 | Similar to ABRE-binding | AT2G46270 (35.9%) | GBF3 had cold treatment induced change in |
| | factor BZ-1 | | expression in Brassica oleracea homolog (110). |
| | | | Increased expression in plants with esk1 |
| | | | mutation, which showed improved freezing |
| | | | tolerance (83). |
| 34 | Similar to Putative cytochrome | AT5G63450 (58.4%) | C. bungeana homolog upregulated by chilling |
| | P450-dependent fatty acid | | stress (69). |
| | hydroxylase | | |
| 35 | PROPROTEIN | Os02g53860 (49.6%) | Putative subtilase 22 identified as an early cold- |
| | CONVERTASE | | regulated protein (111). |
| | SUBTILISIN/KEXIN | | |
| 36 | Nucleobase:cation symporter-1, | AT5G03555 (71.5%) | Upregulated throughout two days of cold |
| | NCS1 family | | treatment (112). |
| 37 | Similar to Putative | AT1G65690 (36.2%) | C. bungeana homolog upregulated by chilling |
| | uncharacterized protein | | stress (69). |
| | | AT2G35980 (37.8%) | Upregulated in acyl-CoA-binding proteins |
| | | | (ACBPs) overexpressing lines. ACBPs are |
| | | | involved in development and stress response |
| | | | (113). |
| | | AT3G11660 (41.3%) | Differentially expressed under freezing or post- |
| | | | freezing recovery (114). |
| 38 | Similar to TRAF-type zinc | Os04g14110 (68.8%) | Identified as having a possible role in cell |
| | finger family protein | | elongation (115). |
| 39 | Similar to Os07g0591300 | Os11g34790 (32.6%) | Increased expression with DaCBF7. CBF7 is a |
| | protein | | transcription factor, D. antarctica C-repeat |
| | | | binding factor 7, in Deschampsia antarctica, an |
| | | | Antarctic hairgrass. Overexpression of DaCBF7 |
| | | | enhanced tolerance to cold stress without growth |
| | | | defects in rice. Os11g34790 contains a putative |
| | | | CRT/DRE or low-temperature responsive |
| | | | elements in the promoter region. (116). |
| 40 | Leucine Rich Repeat (LRR_1) // | AT5G20480 (61.1%) | AT5G20480 has been established as a SERK- |
| | Protein tyrosine kinase | | interacting protein. The somatic embryogenesis |
| | (Pkinase_Tyr) // Leucine rich | | receptor kinases (SERKs) have been |
| | repeat N-terminal domain | | demonstrated to associate with multiple ligand- |
| | (LRRNT_2) // Leucine Rich | | binding receptors to regulate plant growth (117). |
| | repeats (2 copies) (LRR_4) // | | |
| | Leucine rich repeat (LRR_8) | | |
| 41 | AMP-ACTIVATED PROTEIN | AT3G52950 (74.9%) | Identified as a gene that determines the |
| | KINASE, GAMMA | | increasing amount of radial growth resulting |
| | REGULATORY SUBUNIT | | from height growth in Poplar trees (118). |
| 42 | PHD FINGER PROTEIN 20- | AT1G33420 (59.4%) | Identified as a cold regulated transciption factor |
| | RELATED | | that is down regulated in a southern accession |
| | | | association with a higher temperature climate |
| | | | (77). |
| 43 | Similar to receptor-like protein | AT4G20270 (61.9%) | C. bungeana homolog down-regulated by chilling |
| | kinase 3 | | stress (69). |
| | | AT1G75820 (59.4%) | Upregulated in stems undergoing secondary |
| | | | growth (119). |
| | | Os04g04330 (48.9%) | Os04g04330 encodes CTB4a, a gene that confers |
| | | | cold tolerance (72). |
| 44 | Similar to OSIGBa0123D13.3 | AT5G64460 (75.8%) | Identified as an important gene for plant growth |
| | protein | | and reproduction (120). |
| 45 | Pseudo-response regulator 7 | | Ma1, represses flowering in long days in |
| | (PRR7) | | sorghum and was identified as pseudoresponse |
| | | | regulator protein (14). |
| 46 | Similar to H0305E08.5 protein | Os05g30750 (69.8%) | Significantly induced by chilling stress (121). |
| 47 | Similar to Maltose excess | AT5G17520 (67.6%) | mRNA abundance increased in response to low |
| | protein 1-like, chloroplast | | temperatures in plants that overexpressed |
| | precursor | | sucrose-phosphate synthase (94). |
| 48 | Similar to B0403H10- | AT5G26670 (64%) | Pectinesterase induced by chilling (102). |
| | OSIGBa0105A11.19 protein | | |
| 49 | Similar to B0403H10- | AT3G57430 (48.8%) | Identified as a cold regulated gene (39). |
| | OSIGBa0105A11.20 protein | | |
| 50 | Plastocyanin-like domain; | Os08g04310 (54.4%) | Predicted as plastocyanin-like protein (122). |
| | Similar to Os08g0137400 | | Identified as putative plastocyanin-like domain |
| | protein | | containing protein (123). |
| | | AT2G02850 (38%) | Plantacyanin expression reduced in miR408-OX |
| | | | plants. miR408-OX plants exhibit elevated |
| | | | growth vigor, higher fresh weight, and leaves |
| | | | morphologically larger than those of wild-type |
| | | | plants (124). |
| 51 | Similar to Os08g0137400 | AT5G26330 (47.4%) | Exhibited changes in expression following |
| | protein | | induction of VND6, which is involved in xylem |
| | | | formation (125). |
| | | AT2G02850 (39.1%) | Plantacyanin expression reduced in miR408-OX |
| | | | plants. miR408-OX plants exhibit elevated |
| | | | growth vigor, higher fresh weight, and leaves |
| | | | morphologically larger than those of wild-type |
| | | | plants (124). |
| 52 | Similar to Leucine Rich Repeat | AT2G36570 (38.9%) | At2g36570 may be involved in a fundamental |
| | family protein | | cellular process such as cell expansion and |
| | | | elongation. Loss of function of At2g36570 |
| | | | resulted in irregular cell shape, defective vascular |
| | | | bundle lignification, aberrant leaf morphology, |
| | | | and delay in flowering timing (126). |
| | | AT1G48480 (37.5%) | Identified as a cold regulated receptor-like kinase |
| | | | (39). |
| | | | Association with signaling generated from the |
| | | | early cold response (127). |
| 53 | Similar to Laccase-23 | Os12g01730 (72.8%) | Putative gene targeted by miRNA expressed |
| | precursor | | differentially in anthers of two rice cultivars at |
| | | | the UNM stage under cool growth conditions |
| | | | (128). |
| | | Os01g61160 (64.7%) | Upregulated in response to cold stress (121). |
| 54 | Similar to Cytochrome P450 | AT1G13090 (52.3%) | Shows significant changes after 6 hours at 14° C. |
| | | | when compared to the 20° C. (129). |
| | | AT4G31500 (55.8%) | Identified as a cold-affected auxin related gene |
| | | | (39). |
| 55 | Similar to Respiratory burst | AT5G47910 (71.8%) | Affected in the down-regulation by chilling |
| | oxidase | | (102). |
| 56 | Similar to Putative | AT3G53270 (65%) | Up-regulated throughout 2 days of cold treatment |
| | uncharacterized protein | | in lag phase cells (112). |
| | OSJNBa0041P03.7 | | |
| 57 | Similar to Leucine Rich Repeat | AT3G54650 (61.7%) | Loss of FBL17 function reduces plant growth by |
| | family protein | | altering cell division activity in shoot and root |
| | | | apical meristems (130). |
| 58 | Similar to Auxin-responsive | AT3G04730 (63.7%) | iaa16-1 was identified as a dominant gain-of- |
| | protein IAA30 | | function mutation in At3g04730 that restricts |
| | | | growth of adult plants (131). |
| | | | Cassava homolog downregulated with low |
| | | | temperature (132). |
| | | AT3G23050 (65.1%) | Identified as a direct auxin target gene involved |
| | | | in growth and in cross-talk with other signaling |
| | | | pathways (133). |
| | | | Decreased expression in VUP1 OX 9 day old |
| | | | seedlings. Constitutive overexpression of VUP1 |
| | | | caused severe dwarfism (134). |
| | | Os05g14180 (41.3%) | Identified as a growth transcription factor up- |
| | | | regulated by cold-independent overexpression of |
| | | | OsMyb4 (135). |
| | | Os05g48590 (38.8%) | Expression induced by cold stress (104). |
| 59 | Weakly similar to Putative | AT2G01570 (45.9%) | Identified as a repressor of elongation growth |
| | uncharacterized protein | | (136). |
| | | | RGA accumulation enhanced by cold/CBF1. |
| | | | Constitutive expression of CBF1 confers freezing |
| | | | tolerance but also slows growth (137). |
| | | | The GFP:RGA level was enhanced at 12° C. in 8 |
| | | | day old roots (138). |
| | | Os07g36170 (39.2%) | Differentially expressed in OsNPR1-OX |
| | | | compared with the wild-type. OsNPR1 affects |
| | | | rice growth and development (139). |
| 60 | Similar to Chromosome chr6 | Os03g57940 (83.5%) | Interacts with OsACA6, which exhibited cold |
| | scaffold_3, whole genome | | tolerance compared to the wild type and has a |
| | shotgun sequence | | promoter inducible by cold stress (140). |
| 61 | Similar to Os01g0778300 | AT3G13520 (59.7%) | Decreased expression in VUP1 OX 9 day old |
| | protein | | seedlings. Constitutive overexpression of VUP1 |
| | | | caused severe dwarfism (134). |
| | | AT4G26320 (61.3%) | Transcript level of At4g26320 decreased in |
| | | | atnup 160-1 mutant plants. atnup160-1 plants had |
| | | | altered cold-responsive gene expression and |
| | | | sensitivity to chilling stress and were defective in |
| | | | acquired freezing tolerance (107). |
| 62 | | AT1G28280 (50.2%) | AtVQ4 and its Brassica rapa homolog, BrVQ4, |
| | | | were induced by cold and SA treatment. VQ |
| | | | motif-containing proteins in Arabidopsis thaliana |
| | | | and Oryza sativa are known to play an important |
| | | | role in plant growth, development, and stress |
| | | | responses (141). |
| | | AT5G53830 (43.5%) | Identified as a cold-responsive gene |
| | | | downregulated with a difference greater than 1.5- |
| | | | fold by ahk2 ahk3 mutations compared with that |
| | | | of the wildtype. It has been shown that |
| | | | ARABIDOPSIS HISTIDINE KINASE(AHK)2, |
| | | | and AHK3 play roles in cold signaling (142). |
| | | Os05g44270 (55.7%) | Downregualted under drought stress (143). |
| 63 | Similar to Os05g0519200 | GRMZM2G474546 (90.1%) | Differentially expressed in response to drought |
| | protein | | stress (144). |
| 64 | Similar to Putative | AT2G20670 (49.5%) | AT2G20670 is upregulated in atmyb60-1 plants. |
| | uncharacterized protein | | AtMYB60 is negatively modulated during |
| | | | drought and a null mutation in AtMYB60 results |
| | | | in the constitutive reduction of stomatal opening |
| | | | and in decreased wilting under water stress |
| | | | conditions (145). |
| | | AT2G38820 (32.9%) | P. euphratica homolog upregulated in drought- |
| | | | stressed leaves (146). |
| 65 | Similar to Putative | AT4G23630 (60.2%) | Showed significant changes in expression after |
| | uncharacterized protein | | 6 hours at 14° C. when compared to 20° C. (129). |
| 66 | Similar to Putative | Os10g36160 (44.3%) | Identified as a cold related gene (147). |
| | uncharacterized protein | | |
| 67 | PROTEIN KINASE-LIKE | Os04g29680 (35.7%) | Differentially expressed in SNAC2 |
| | PROTEIN-RELATED | | overexpressed rice plants. SNAC2 confers cold |
| | | | tolerance (87). |
| 68 | Similar to Os01g0658700 | AT5G66530 (47.8%) | Downregulated in winter cultivars of barley, but |
| | protein | | not spring cultivars (148). |
| 69 | GALACTINOL--SUCROSE | AT5G20250 (72%) | Showed significant changes after 6 h at 14° C. |
| | GALACTOSYLTRANSFERASE | | when compared to the 20° C. (129). |
| | 6-RELATED | | Leaf enzyme in winter barley homolog is |
| | | | differentially expressed after 3 days at 3° C. (63). |
| 70 | Similar to Os02g0756800 | AT1G35140 (60.4%) | Downregulated in plants overexpressing YFP- |
| | protein | | SDG714. Overexpression of YFP-SDG714 |
| | | | inhibits plant growth (149). |
| | | | Upregulated in AtCSP3-overexpressing plants. |
| | | | AtCSP3 is a cold shock domain protein (65). |
| | | AT2G17230 (49.1%) | Vitis vinifera homolog had increased expression |
| | | | in lines overexpressing CBF4 and there was |
| | | | increased expression of At1g35140 in Poplar |
| | | | overexpressing CBF4. CBF4 overexpression |
| | | | resulted in increased freezing tolerance and |
| | | | reduced growth phenotypes (150). |
| 71 | Similar to Putative | Os06g11720 (69.3%) | Differentially expressed in OsNPR1-OX |
| | glucosyltransferase-10 | | compared with the wild-type. OsNPR1 affects |
| | | | rice growth and development (139). |
| 72 | Similar to Os06g0695800 | AT2G47000 (48.1%) | TWISTED DWARF1 (TWD1) acts as a |
| | protein | | chaperone of ABCB4 (At2g47000) and ABCB4 |
| | | | is delocalized and degraded in twd1. Polar auxin |
| | | | transport is reduced in twd1, causing dwarfism |
| | | | and disoriented growth (151). |
| | | Os01g50100 (45.9%) | Multidrug resistance protein 4 is upregulated by |
| | | | cold and by overexpression of MYBS3. MYBS3 |
| | | | has been shown to be necessary for cold |
| | | | tolerance in rice (152). |

The breadth and scope of the present disclosure should not be limited by any of the above-described exemplary embodiments, but should be defined only in accordance with the following claims and their equivalents.

The present invention can be defined in any of the following numbered paragraphs:
1. A method for obtaining a sorghum plant comprising in its genome at least one cold-stress tolerance genetic locus, the method comprising the steps of:
   a. genotyping a plurality of sorghum plants with respect to at least one genetic locus comprising the plastocyanin gene Sobic.007G033300 (SEQ ID NO: 1); and
   b. selecting a sorghum plant comprising in its genome at least one genetic locus comprising a genotype associated with cold-stress tolerance, based on said genotyping.
2. The method of paragraph 1, wherein the genetic locus is a genomic region between any of 1 Mb, 500 kb, 100 kb, 50 kb, or 10 kb telomere proximal and any of 1 Mb, 500 kb, 100 kb, 50 kb, or 10 kb centromere proximal of the plastocyanin gene Sobic.007G033300.
3. The method of paragraph 1, wherein the genetic locus consists of the plastocyanin gene Sobic.007G033300.
4. The method of paragraph 1,
   wherein the genotype associated with cold-stress tolerance comprises at least one polymorphic allele associated with cold-stress tolerance of at least one marker in the gene Sobic.007G033300 (SEQ ID NO: 1) selected from the group consisting of Chr07:2934702, Chr07:2934028, Chr07:2934031, Chr07:2934040, Chr07:2934099, Chr07:2934128, Chr07:2934129, Chr07:2934187, and Chr07:2934291; or
   wherein the genotype associated with cold-stress tolerance comprises a genetically edited variant of the plastocyanin gene Sobic.007G033300.
5. The method of paragraph 4, wherein the genotype associated with cold-stress tolerance comprises at least two, at least three, at least four, at least five, at least six, at least seven, at least eight, or at least nine of said polymorphic alleles.
6. The method of paragraph 4, wherein the genotype associated with cold-stress tolerance comprises at least one polymorphic allele associated with cold-stress tolerance of at least the marker Chr07.2934702.
7. The method of paragraph 1, wherein the genotype associated with cold-stress tolerance comprises at least one single nucleotide polymorphism (SNP) associated with cold-stress tolerance of at least one marker in Sobic.007G033300 (SEQ ID NO: 1), wherein the SNP is selected from the group consisting of a G allele of SNP 7:2934702, a C allele of SNP 7:2934028, a G allele of SNP 7:2934031, a T allele of SNP 7:2934040, a C allele of SNP 7:2934099, an A allele of 7:2934128, a G allele of 7:2934129, a T allele of SNP 07:2934187, and a C allele replacing the nucleotides CCCG beginning at position 07:2934291.
8. The method of paragraph 7, wherein the genotype associated with cold-stress tolerance comprises at least two, at least three, at least four, at least five, at least six, at least seven, at least eight, or at least nine of said SNPs.
9. The method of paragraph 7, wherein the genotype associated with cold-stress tolerance comprises a G allele of SNP 7:2934702.
10. The method of any one of paragraphs 1 to 9, wherein said selected sorghum plant or a progeny thereof exhibits cold-stress tolerance.
11. The method of any one of paragraphs 1 to 10, further comprising crossing the selected sorghum plant having in its genome a cold-stress tolerance genetic locus with a cold-stress sensitive sorghum plant to produce a progeny sorghum plant comprising in its genome at least one cold-stress tolerance genetic locus.
12, The method of paragraph 11, wherein the progeny sorghum plant exhibits cold-stress tolerance.
13. The method of paragraph 11, wherein the selected sorghum plant having in its genome a cold-stress tolerance genetic locus is crossed with a cold-stress sensitive sorghum plant to produce a population of sorghum plants comprising in their genomes at least one cold-stress tolerance genetic locus.
14. The method of paragraph 13, wherein the population of sorghum plants produced comprises a plurality of sorghum plants that exhibit cold-stress tolerance.
15. The method of paragraph 14, wherein at least 50%, at least 60%, at least 70%, at least 75%, at least 80%, at least 85%, at least 90%, at least 95%, at least 96%, at least 97%, at least 98%, at least 99%, or 100% of the sorghum plants in the population of sorghum plants produced exhibit cold-stress tolerance.
16. The method of any one of paragraphs 1 to 15, wherein the plurality of sorghum plants genotyped in (a) comprises a population that is obtained by:
   i) crossing a parent plant comprising at least one cold-stress tolerance locus with a parent plant comprising at least one non-cold-stress tolerance locus; or
   ii) obtaining seed or progeny from a parental plant segregating for at least one cold-stress tolerance locus.
17. The method of any one of paragraphs 1 to 16, wherein the plurality of sorghum plants genotyped in (a) comprises a population of plants that:
   (i) are non-transgenic and resistant to a herbicide;
   (ii) contain a transgene that confers resistance to a herbicide; or
   (iii) contain a transgene that confers resistance to a herbicide and are resistant to an additional herbicide to which the plant does not contain a transgene conferring resistance thereto.
18. The method of paragraph 17, wherein the herbicide is glyphosate, dicamba, and/or imidazoline.
19. The method of any one of paragraphs 1 to 18, further comprising the step of genotyping for the presence of at least one additional marker, wherein said additional marker is either linked or unlinked to said genomic locus comprising a plastocyanin gene Sobic.007G033300 (SEQ ID NO: 1), optionally wherein said additional marker is linked.
20. The method of any one of paragraphs 1 to 19, wherein said method further comprises exposing the selected sorghum plant or a progeny thereof comprising the cold-stress tolerance genetic locus to cold conditions sufficient to cause a deleterious effect in a non-cold-stress tolerant variety and/or an amount of herbicide sufficient to cause a deleterious effect in a sorghum plant that is not resistance to the herbicide.
21. A method for producing a sorghum plant comprising in its genome at least one introgressed cold-stress tolerance genetic locus, the method comprising the steps of:
   a. crossing a first sorghum plant with a genotype associated with cold-stress tolerance in a genetic locus comprising a plastocyanin gene Sobic.007G033300 with a second sorghum plant comprising a genotype not associated with cold-stress tolerance in a genetic locus comprising a plastocyanin gene Sobic.007G033300 and at least one second polymorphic locus that is linked to the genetic locus comprising Sobic.007G033300 and that is not present in said first sorghum plant to obtain a population segregating for the cold-stress tolerance loci and said linked polymorphic locus;
   b. genotyping for the presence of at least two polymorphic nucleic acids in at least one sorghum plant from said population, wherein a first polymorphic nucleic acid is located in said genetic locus comprising Sobic.007G033300 and wherein a second polymorphic amino acid is a linked polymorphic locus not present in said first sorghum plant; and
   c. selecting a sorghum plant comprising a genotype associated with cold-stress tolerance and at least one linked marker found in said second sorghum plant comprising a non-cold-stress tolerance locus but not found in said first sorghum plant, thereby obtaining a sorghum plant comprising in its genome an introgressed cold-stress tolerance locus.
22. The method of paragraph 21, wherein the genetic locus is a genomic region between any of 1 Mb, 500 kb, 100 kb, 50 kb, or 10 kb telomere proximal and any of 1 Mb, 500 kb, 100 kb, 50 kb, or 10 kb centromere proximal of a plastocyanin gene Sobic.007G033300.
23. The method of paragraph 21, wherein the genetic locus consists of a plastocyanin gene Sobic.007G033300.
24. The method of paragraph 21,
   wherein the genotype associated with cold-stress tolerance comprises at least one polymorphic allele associated with cold-stress tolerance of at least one marker in the gene Sobic.007G033300 (SEQ ID NO: 1) selected from the group consisting of Chr07:2934702, Chr07:2934028, Chr07:2934031, Chr07:2934040, Chr07:2934099, Chr07:2934128, Chr07:2934129, Chr07:2934187, and Chr07:2934291; or
   wherein the genotype associated with cold-stress tolerance comprises a genetically edited variant of the plastocyanin gene Sobic.007G033300.
25. The method of paragraph 24, wherein the genotype associated with cold-stress tolerance comprises at least two, at least three, at least four, at least five, at least six, at least seven, at least eight, or at least nine of said polymorphic alleles.
26. The method of paragraph 24, wherein the genotype associated with cold-stress tolerance comprises at least one polymorphic allele associated with cold-stress tolerance of at least the marker Chr07.2934702 in the gene Sobic.007G033300 (SEQ ID NO: 1).
27. The method of paragraph 21, wherein the genotype associated with cold-stress tolerance comprises at least one single nucleotide polymorphism (SNP) associated with cold-stress tolerance of at least one marker in Sobic.007G033300 (SEQ ID NO: 1), wherein the SNP is selected from the group consisting of a G allele of SNP 7:2934702, a C allele of SNP 7:2934028, a G allele of SNP 7:2934031, a T allele of SNP 7:2934040, a C allele of SNP 7:2934099, an A allele of 7:2934128, a G allele of 7:2934129, a T allele of SNP 07:2934187, and a C allele replacing the nucleotides CCCG beginning at position 07:2934291.
28. The method of paragraph 27, wherein the genotype associated with cold-stress tolerance comprises at least two, at least three, at least four, at least five, at least six, at least seven, at least eight, or at least nine of said SNPs.
29. The method of paragraph 27, wherein said genotype associated with cold-stress tolerance comprises a G allele of SNP 7:2934702.
30. The method of any one of paragraphs 21 to 29, wherein said sorghum plant comprising in its genome at least one introgressed cold-stress tolerance locus exhibits cold-stress tolerance.
31. The method of any one of paragraphs 21 to 30, wherein at least one of said first or said second sorghum plants:
   (i) is non-transgenic and resistant to a herbicide;
   (ii) contains a transgene that confers resistance to a herbicide; or
   (iii) contains a transgene that confers resistance to a herbicide and is resistant to an additional herbicide to which the plant does not contain a transgene conferring resistance thereto.
32. The method of any one of paragraphs 21 to 31, wherein said method further comprises exposing the selected sorghum plant or progeny thereof comprising the cold-stress tolerance genetic locus to cold conditions sufficient to cause a deleterious effect in a non-cold-stress tolerant variety and/or an amount of herbicide sufficient to cause a deleterious effect in a sorghum plant that is not resistance to the herbicide.
33. The method of any one of paragraphs 21 to 32, wherein said second linked polymorphic locus is detected with a marker that is located within about 1 Mb, 500, 100, 40, 20, 10, or 5 kilobases (Kb) of said cold-stress tolerance locus.
34. A method of identifying a sorghum plant that comprises a genotype associated with cold-stress tolerance, comprising:
   genotyping a sorghum plant for the presence of an allele in at least one genetic locus associated with cold-stress tolerance, wherein the genetic locus comprises a plastocyanin gene Sobic.007G033300 (SEQ ID NO: 1), and denoting based on the genotyping that said sorghum plant comprises a genotype associated with cold-stress tolerance.
35. The method of paragraph 34, wherein said method further comprises the step of selecting said denoted plant from a population of plants.
36. The method of paragraph 34 or 35, further comprising the step of exposing the identified and/or selected sorghum plant or progeny thereof comprising the genotype associated with cold-stress tolerance to cold conditions sufficient to cause a deleterious effect in a non-cold-stress tolerant variety.
37. The method of any one of paragraphs 34 to 36, wherein said sorghum plant comprising in its genome at least one introgressed cold-stress tolerance genetic locus exhibits cold-stress tolerance.
38. The method of any one of paragraphs 34 to 37, wherein the genetic locus is a genomic region between any of 1 Mb, 500 kb, 100 kb, 50 kb, or 10 kb telomere proximal and any of 1 Mb, 500 kb, 100 kb, 50 kb, or 10 kb centromere proximal of a plastocyanin gene Sobic.007G033300.
39. The method of any one of paragraphs 34 to 37, wherein the genetic locus consists of a plastocyanin gene Sobic.007G033300.
40. The method of any one of paragraphs 34 to 39,
   wherein the genotype associated with cold-stress tolerance comprises at least one polymorphic allele associated with cold-stress tolerance of at least one marker in the gene Sobic.007G033300 (SEQ ID NO: 1) selected from the group consisting of Chr07:2934702, Chr07:2934028, Chr07:2934031, Chr07:2934040, Chr07:2934099, Chr07:2934128, Chr07:2934129, Chr07:2934187, and Chr07:2934291; or
   wherein the genotype associated with cold-stress tolerance comprises a genetically edited variant of the plastocyanin gene Sobic.007G033300.
41. The method of paragraph 40, wherein the genotype associated with cold-stress tolerance comprises at least two, at least three, at least four, at least five, at least six, at least seven, at least eight, or at least nine of said polymorphic alleles.
42. The method of paragraph 40, wherein the genotype associated with cold-stress tolerance comprises at least one polymorphic allele associated with cold-stress tolerance of at least the marker Chr07.2934702 in the gene Sobic.007G033300 (SEQ ID NO: 1).
43. The method of any one of paragraphs 34 to 39, wherein the genotype associated with cold-stress tolerance comprises at least one single nucleotide polymorphism (SNP) associated with cold-stress tolerance of at least one marker in Sobic.007G033300 (SEQ ID NO: 1), wherein the SNP is selected from the group consisting of a G allele of SNP 7:2934702, a C allele of SNP 7:2934028, a G allele of SNP 7:2934031, a T allele of SNP 7:2934040, a C allele of SNP 7:2934099, an A allele of 7:2934128, a G allele of 7:2934129, a T allele of SNP 07:2934187, and a C allele replacing the nucleotides CCCG beginning at position 07:2934291.
44. The method of paragraph 43, wherein the genotype associated with cold-stress tolerance comprises at least two, at least three, at least four, at least five, at least six, at least seven, at least eight, or at least nine of said SNPs.
45. The method of paragraph 43, wherein said genotype associated with cold-stress tolerance comprises a G allele of SNP 7:2934702.
46. A sorghum plant made by the method of any one of paragraphs 21 to 33, wherein said sorghum plant comprises an introgressed cold-stress tolerance locus.
47. A sorghum plant comprising an introgressed cold-stress tolerance genetic locus comprising a genotype associated with cold-stress tolerance in a genomic region comprising a plastocyanin gene Sobic.007G033300, wherein at least one marker linked to the introgressed cold-stress tolerance genetic locus found in said sorghum plant is characteristic of germplasm comprising a non-cold-stress tolerance genetic locus but is not associated with germplasm comprising the cold-stress tolerance genetic locus.
48. The sorghum plant of paragraph 47, wherein the genetic locus is a genomic region between any of 1 Mb, 500 kb, 100 kb, 50 kb, or 10 kb telomere proximal and any of 1 Mb, 500 kb, 100 kb, 50 kb, or 10 kb centromere proximal of a plastocyanin gene Sobic.007G033300.
49. The sorghum plant of paragraph 47, wherein the genetic locus consists of a plastocyanin gene Sobic.007G033300.
50. The sorghum plant of paragraph 47,
   wherein the genotype associated with cold-stress tolerance comprises at least one polymorphic allele associated with cold-stress tolerance of at least one marker in the gene Sobic.007G033300 (SEQ ID NO: 1) selected from the group consisting of Chr07:2934702, Chr07:2934028, Chr07:2934031, Chr07:2934040, Chr07:2934099, Chr07:2934128, Chr07:2934129, Chr07:2934187, and Chr07:2934291; or
   wherein the genotype associated with cold-stress tolerance comprises a genetically edited variant of the plastocyanin gene Sobic.007G033300.
51. The sorghum plant of paragraph 50, wherein the genotype associated with cold-stress tolerance comprises at least two, at least three, at least four, at least five, at least six, at least seven, at least eight, or at least nine of said polymorphic alleles.
52. The sorghum plant of paragraph 50, wherein the genotype associated with cold-stress tolerance comprises at least one polymorphic allele associated with cold-stress tolerance of at least the marker Chr07.2934702 in the gene Sobic.007G033300 (SEQ ID NO: 1).
53. The sorghum plant of paragraph 47, wherein the genotype associated with cold-stress tolerance comprises at least one single nucleotide polymorphism (SNP) associated with cold-stress tolerance of at least one marker in Sobic.007G033300 (SEQ ID NO: 1), wherein the SNP is selected from the group consisting of a G allele of SNP 7:2934702, a C allele of SNP 7:2934028, a G allele of SNP 7:2934031, a T allele of SNP 7:2934040, a C allele of SNP 7:2934099, an A allele of 7:2934128, a G allele of 7:2934129, a T allele of SNP 07:2934187, and a C allele replacing the nucleotides CCCG beginning at position 07:2934291.
54. The sorghum plant of paragraph 53, wherein the genotype associated with cold-stress tolerance comprises at least two, at least three, at least four, at least five, at least six, at least seven, at least eight, or at least nine of said SNPs.
55. The sorghum plant of paragraph 53, wherein said genotype associated with cold-stress tolerance comprises a G allele of SNP 7:2934702.
56. The sorghum plant of any one of paragraphs 46 to 55, wherein the sorghum plant or a progeny thereof exhibits cold-stress tolerance.
57. A method of producing a commercial crop seed lot of sorghum seeds comprising in their genomes at least one introgressed cold-stress tolerance genetic locus, the method comprising the steps of:
   a. producing a population of sorghum plants from the sorghum plant selected in step (c) of any one of paragraphs 21 to 33 or the sorghum plant of any one of paragraphs 46 to 56 comprising a genotype associated with cold-stress tolerance and at least one linked marker found in said second sorghum plant comprising a non-cold-stress tolerance locus but not found in said first sorghum plant; and
   b. harvesting a commercial seed lot, wherein the harvested crop seed lot comprises a plurality of seeds that comprise in their genomes at least one introgressed cold-stress tolerance locus.
58. The method of paragraph 57, wherein the genetic locus is a genomic region between any of 1 Mb, 500 kb, 100 kb, 50 kb, or 10 kb telomere proximal and any of 1 Mb, 500 kb, 100 kb, 50 kb, or 10 kb centromere proximal of a plastocyanin gene Sobic.007G033300.
59. The method of paragraph 57, wherein the genetic locus consists of a plastocyanin gene Sobic.007G033300.
60. The method of paragraph 57,
   wherein the genotype associated with cold-stress tolerance comprises at least one polymorphic allele associated with cold-stress tolerance of at least one marker in the gene Sobic.007G033300 (SEQ ID NO: 1) selected from the group consisting of Chr07:2934702, Chr07:2934028, Chr07:2934031, Chr07:2934040, Chr07:2934099, Chr07:2934128, Chr07:2934129, Chr07:2934187, and Chr07:2934291; or
   wherein the genotype associated with cold-stress tolerance comprises a genetically edited variant of the plastocyanin gene Sobic.007G033300.
61. The method of paragraph 60, wherein the genotype associated with cold-stress tolerance comprises at least two, at least three, at least four, at least five, at least six, at least seven, at least eight, or at least nine of said polymorphic alleles.
62. The method of paragraph 60, wherein the genotype associated with cold-stress tolerance comprises at least one polymorphic allele associated with cold-stress tolerance of at least the marker Chr07.2934702 in the gene Sobic.007G033300 (SEQ ID NO: 1).
63. The method of paragraph 57, wherein the genotype associated with cold-stress tolerance comprises at least one single nucleotide polymorphism (SNP) associated with cold-stress tolerance of at least one marker in Sobic.007G033300 (SEQ ID NO: 1), wherein the SNP is selected from the group consisting of a G allele of SNP 7:2934702, a C allele of SNP 7:2934028, a G allele of SNP 7:2934031, a T allele of SNP 7:2934040, a C allele of SNP 7:2934099, an A allele of 7:2934128, a G allele of 7:2934129, a T allele of SNP 07:2934187, and a C allele replacing the nucleotides CCCG beginning at position 07:2934291.
64. The method of paragraph 63, wherein the genotype associated with cold-stress tolerance comprises at least two, at least three, at least four, at least five, at least six, at least seven, at least eight, or at least nine of said SNPs.
65. The method of paragraph 63, wherein said genotype associated with cold-stress tolerance comprises a G allele of SNP 7:2934702.
66. The method of paragraph any one of paragraphs 57 to 65, wherein the seed lot comprise at least 100 seeds, at least 500 seeds, at least 1,000 seeds, at least 5,000 seeds, at least 10,000 seeds, at least 25,000 seeds, at least 50,000 seeds, or at least 100,000 seeds.
67. The method of any one of paragraphs 57 to 66, wherein the plurality of seeds that comprise in their genomes at least one introgressed cold-stress tolerance genetic locus constitute at least 85%, at least 90%, at least 91%, at least 92%, at least 93%, at least 94%, at least 95%, at least 96%, at least 97%, at least 98%, or at least 99% of the seed of the harvested seed lot.
68. The method of any one of paragraphs 57 to 67, wherein the method further comprises the step of packaging seed obtained from said seed lot into one or more bags to obtain one or more packaged seed bags.
69. The method of paragraph 68, wherein said method further comprises the step of distributing the packaged seeds to growers for use in crop production.
70. A commercial crop seed lot of sorghum seeds made by the method of any one of paragraphs 57 to 69.
71. A commercial crop seed lot of sorghum seeds comprising a plurality of seeds that comprise in their genomes at least one introgressed cold-stress tolerance genetic locus comprising a genotype associated with cold-stress tolerance.
72. The seed lot of paragraph 71, wherein the genetic locus is a genomic region between any of 1 Mb, 500 kb, 100 kb, 50 kb, or 10 kb telomere proximal and any of 1 Mb, 500 kb, 100 kb, 50 kb, or 10 kb centromere proximal of a plastocyanin gene Sobic.007G033300.
73. The seed lot of paragraph 71, wherein the genetic locus consists of a plastocyanin gene Sobic.007G033300.
74. The seed lot of paragraph 71,
   wherein the genotype associated with cold-stress tolerance comprises at least one polymorphic allele associated with cold-stress tolerance of at least one marker in the gene Sobic.007G033300 (SEQ ID NO: 1) selected from the group consisting of Chr07:2934702, Chr07:2934028, Chr07:2934031, Chr07:2934040, Chr07:2934099, Chr07:2934128, Chr07:2934129, Chr07:2934187, and Chr07:2934291; or
   wherein the genotype associated with cold-stress tolerance comprises a genetically edited variant of the plastocyanin gene Sobic.007G033300.
75. The seed lot of paragraph 74, wherein the genotype associated with cold-stress tolerance comprises at least two, at least three, at least four, at least five, at least six, at least seven, at least eight, or at least nine of said polymorphic alleles.
76. The seed lot of paragraph 74, wherein the genotype associated with cold-stress tolerance comprises at least one polymorphic allele associated with cold-stress tolerance of at least the marker Chr07.2934702 in the gene Sobic.007G033300 (SEQ ID NO: 1).
77. The seed lot of paragraph 71, wherein the genotype associated with cold-stress tolerance comprises at least one single nucleotide polymorphism (SNP) associated with cold-stress tolerance of at least one marker in Sobic.007G033300 (SEQ ID NO: 1), wherein the SNP is selected from the group consisting of a G allele of SNP 7:2934702, a C allele of SNP 7:2934028, a G allele of SNP 7:2934031, a T allele of SNP 7:2934040, a C allele of SNP 7:2934099, an A allele of 7:2934128, a G allele of 7:2934129, a T allele of SNP 07:2934187, and a C allele replacing the nucleotides CCCG beginning at position 07:2934291.
78. The seed lot of paragraph 77, wherein the genotype associated with cold-stress tolerance comprises at least two, at least three, at least four, at least five, at least six, at least seven, at least eight, or at least nine of said SNPs.
79. The seed lot of paragraph 77, wherein said genotype associated with cold-stress tolerance comprises a G allele of SNP 7:2934702.
80. The seed lot of any one of paragraphs 70 to 79, wherein the seed lot comprise at least 100 seeds, at least 500 seeds, at least 1,000 seeds, at least 5,000 seeds, at least 10,000 seeds, at least 25,000 seeds, at least 50,000 seeds, or at least 100,000 seeds.
81. The seed lot of any one of paragraphs 70 to 80, wherein the plurality of seeds that comprise in their genomes at least one introgressed cold-stress tolerance genetic locus constitute at least 85%, at least 90%, at least 91%, at least 92%, at least 93%, at least 94%, at least 95%, at least 96%, at least 97%, at least 98%, or at least 99% of the seed of the seed lot.
82. A method of growing a sorghum plant comprising in its genome at least one cold-stress tolerance genetic locus of any one of the preceding paragraphs, the method comprising growing the plant under cold conditions sufficient to cause a deleterious effect in a non-cold-stress tolerant variety.
83. The method of paragraph 82, wherein said plant is grown in an open field.
84. The method of paragraph 82 or 83, wherein said method comprises growing the selected sorghum plant, progeny thereof, or population produced therefrom in the month of February, March, or April.
85. The method of paragraph 84, wherein said method comprises growing the selected sorghum plant, progeny thereof, or population produced therefrom in the month April.
86. The method of paragraph 82 or 83, wherein the method comprises growing the selected sorghum plant, progeny thereof, or population produced therefrom in the northern portion of the continental United States, in USDA Plant Hardiness Zones 5, 4, 3, 2, or 1, and/or in Canada.
87. The method of paragraph 86, wherein said method comprises growing the selected sorghum plant, progeny thereof, or population produced therefrom in the month of April or May.
88. The method of paragraph 86, wherein said method comprises growing the selected sorghum plant, progeny thereof, or population produced therefrom in the month of April.
89. A genetically edited plastocyanin gene comprising:
   (i) a variant polynucleotide encoding a plastocyanin protein variant or fragment thereof,
   wherein the variant polynucleotide comprises at least one nucleotide insertion, deletion, and/or substitution in comparison to the corresponding unedited wild-type polynucleotide sequence, and
   wherein the variant polynucleotide does not encode a wild-type plastocyanin protein,
   optionally, wherein said variant polynucleotide is operably linked to a polynucleotide comprising a promoter;
   (ii) a variant polypeptide comprising a plastocyanin gene 3′ UTR,
   wherein the variant polynucleotide comprises at least one nucleotide insertion, deletion, and/or substitution in the 3′ UTR in comparison to the corresponding unedited wild-type polynucleotide sequence,
   optionally wherein said variant polynucleotide is operably linked to a polynucleotide comprising a promoter and/or a plastocyanin protein coding region;
   (iii) a variant polypeptide comprising a plastocyanin gene 5′ UTR,
   wherein the variant polynucleotide comprises at least one nucleotide insertion, deletion, and/or substitution in the 5′ UTR in comparison to the corresponding unedited wild-type polynucleotide sequence,
   optionally wherein said variant polynucleotide is operably linked to a polynucleotide comprising a promoter and/or a plastocyanin protein coding region;
   (iv) a variant polypeptide comprising a plastocyanin gene promoter,
   wherein the variant polynucleotide comprises at least one nucleotide insertion, deletion, and/or substitution in the promoter in comparison to the corresponding unedited wild-type polynucleotide sequence,
   optionally, wherein said variant polynucleotide is operably linked to a polynucleotide comprising a plastocyanin protein coding region;
   (v) a variant polypeptide comprising a plastocyanin gene intron,
   wherein the variant polynucleotide comprises at least one nucleotide insertion, deletion, and/or substitution in the intron in comparison to the corresponding unedited wild-type polynucleotide sequence,
   optionally, wherein said variant polynucleotide is operably linked to a polynucleotide comprising at least one plastocyanin gene exon; and/or
   (vi) a variant polypeptide encoding a plastocyanin (a) transit peptide, vacuolar targeting peptide, and/or endoplasmic reticulum targeting peptide; (b) plastid targeting peptide; and/or (c) polyadenylation or transcriptional termination signal,
   wherein the variant polynucleotide comprises at least one nucleotide insertion, deletion, and/or substitution in (a), (b), and/or (c) in comparison to the corresponding unedited wild-type polynucleotide sequence,
   optionally, wherein the polynucleotides of (a), (b), and/or (c) are operably linked to a polypeptide encoding a plastocyanin protein.
90. The edited plastocyanin gene of paragraph 89 comprising a variant polynucleotide encoding a plastocyanin protein variant or fragment thereof,
   wherein the variant polynucleotide comprises at least one nucleotide insertion, deletion, and/or substitution in comparison to the corresponding unedited wild-type polynucleotide sequence, and
   wherein the variant polynucleotide does not encode a wild-type plastocyanin protein.
91. The edited plastocyanin gene of paragraph 90, wherein nucleotide insertion, deletion, and/or substitution results in a frameshift mutation and/or a nonsense mutation.
92. The edited plastocyanin gene of paragraph 90 or 91, wherein nucleotide insertion, deletion, and/or substitution is an exon of the edited plastocyanin gene;
   optionally, wherein nucleotide insertion, deletion, and/or substitution is Exon 1 of the edited plastocyanin gene;
   optionally, wherein the variant polynucleotide encodes a plastocyanin protein variant or fragment thereof comprising the amino acid sequence of SEQ ID NO: 8 or SEQ ID NO: 10;
   optionally, wherein the variant polynucleotide comprises the nucleic acid sequence of SEQ ID NO: 7 or SEQ ID NO: 9.
93. The edited plastocyanin gene of any one of paragraphs 90 to 92, wherein the variant polynucleotide encodes a plastocyanin protein variant or fragment thereof having reduced activity in comparison to the corresponding wild-type plastocyanin protein.
94. The edited plastocyanin gene of paragraph 89 comprising a variant polypeptide comprising a plastocyanin gene 3′ UTR,
   wherein the variant polynucleotide comprises at least one nucleotide insertion, deletion, and/or substitution in the 3′ UTR in comparison to the corresponding unedited wild-type polynucleotide sequence;
   optionally, wherein the variant polypeptide comprising a plastocyanin gene 3′ UTR comprises SEQ ID NO: 11;
   optionally, wherein said variant polynucleotide is operably linked to a polynucleotide comprising a promoter and a plastocyanin protein coding region.
95. The edited plastocyanin gene of paragraph 94, wherein the insertion, deletion, and/or substitution in the 3′ UTR results in reduced plastocyanin activity in comparison to an otherwise identical plastocyanin gene having a wild-type 3′ UTR.
96. The edited plastocyanin gene of any one of paragraphs 89 to 95, wherein the variant polynucleotide is integrated into the nuclear or plastid genome of a cell.
97. A plant nuclear or plastid genome comprising the edited plastocyanin gene of any one of paragraphs 89 to 96;
   optionally, wherein the variant polynucleotide is heterologous to the nuclear or plastid genome.
98. The plant nuclear or plastid genome of paragraph 97, wherein said variant polynucleotide is operably linked to an endogenous promoter of the nuclear or plastid genome.
99. The edited gene or the nuclear or plastid genome of any one of paragraphs 89 to 98, further comprising a polynucleotide encoding (i) a transit peptide, a vacuolar targeting peptide, and/or an endoplasmic reticulum targeting peptide; (ii) a plastid targeting peptide; and/or (iii) a polyadenylation or transcriptional termination signal, wherein the polynucleotides of (i), (ii), and/or (iii) are operably linked to the polypeptide encoding the plastocyanin protein.
100. The edited gene or the nuclear or plastid genome of any one of paragraphs 89 to 99,
   wherein the nuclear or plastid genome is a monocot crop plant or a dicot crop plant nuclear or plastid genome and/or wherein the edited plastocyanin gene is from a monocot crop plant or a dicot crop plant; or
   wherein the nuclear or plastid genome is a C3 plant or a C4 plant nuclear or plastid genome and/or wherein the edited plastocyanin gene is from a C3 plant or a C4 plant.
101. The edited gene or the nuclear or plastid genome of paragraph 100, wherein the monocot crop plant nuclear or plastid genome is selected from the group consisting of a corn, barley, oat, pearl millet, rice, sorghum, sugarcane, turf grass, and wheat plant nuclear or plastid genome and/or wherein the edited plastocyanin gene is selected from the group consisting of a corn, barley, oat, pearl millet, rice, sorghum, sugarcane, turf grass, and wheat plant edited plastocyanin gene.
102. A cell comprising the edited gene or nuclear or plastid genome of any one of paragraphs 89 to 101.
103. The cell of paragraph 102, wherein the cell is a plant, yeast, mammalian, or bacterial cell.
104. The cell of paragraph 103, wherein the cell is a plant cell that is non-regenerable.
105. A plant comprising the edited gene or nuclear or plastid genome of any one of paragraphs 89 to 111; optionally, wherein the plant is a monocot crop plant or a dicot crop plant.
106. The plant of paragraph 105, wherein the edited gene or nuclear or plastid genome confers to the plant cold-stress tolerance in comparison to a control plant that lacks the edited gene or nuclear or plastid genome.
107. The plant of paragraph 105 or 106, wherein plant is a monocot crop plant is selected from the group consisting of a corn, barley, oat, pearl millet, rice, sorghum, sugarcane, turf grass, and wheat plant.
108. A plant part of the plant of any one of paragraphs 105 to 107, wherein the plant part comprises the edited gene or nuclear or plastid genome.
109. The plant part of paragraph 108, wherein the plant part is a seed, stem, leaf, root, tuber, flower, or fruit.
110. A method for obtaining a plant comprising the edited gene or plant nuclear or plastid genome of any one of paragraphs 89 to 101, wherein the plant is cold-stress tolerant, the method comprising the steps of:
   (i) introducing the edited gene, the variant polynucleotide encoding the plastocyanin protein, the polynucleotide comprising the promoter, a fragment of said polynucleotides, or a combination of said polynucleotides into a plant cell, tissue, plant part, or whole plant;
   (ii) obtaining a plant cell, tissue, part, or whole plant wherein the edited gene, the variant polynucleotide encoding the plastocyanin protein, the polynucleotide comprising the promoter, a fragment of said polynucleotides, or a combination of said polynucleotides is integrated into the plant nuclear or plastid genome; and
   (iii) selecting a plant obtained from the plant cell, tissue, part or whole plant of step (ii) for expression of a variant plastocyanin protein, thereby obtaining a plant that is cold-stress tolerant.
111. A method for obtaining a plant comprising the edited gene or plant nuclear or plastid genome of any one of paragraphs 89 to 101, wherein the plant is cold-stress tolerant, the method comprising introducing into a plant cell one or more gene editing molecules that target an endogenous plastocyanin gene to introduce at least one nucleotide insertion, deletion, and/or substitution into the endogenous plastocyanin gene.
112. The method of paragraph 111, further comprising selecting a plant comprising a gene edited plastocyanin gene that expresses a variant plastocyanin protein; optionally, further comprising selecting a plant that is cold-stress tolerant.
113. A method for obtaining a plant comprising the edited gene or nuclear or plastid genome of any one of paragraphs 89 to 101, wherein the plant is cold-stress tolerant, comprising the steps of:
   (i) providing to a plant cell, tissue, part, or whole plant an endonuclease or an endonuclease and at least one guide RNA, wherein the endonuclease or guide RNA and endonuclease can form a complex that can introduce a double strand break at a target site in a nuclear or plastid genome of the plant cell, tissue, part, or whole plant;
   (ii) obtaining a plant cell, tissue, part, or whole plant wherein at least one nucleotide insertion, deletion, and/or substitution has been introduced into the corresponding wild-type polynucleotide sequence; and
   (iii) selecting a plant obtained from the plant cell, tissue, part or whole plant of step (ii) comprising the edited gene for expression of a plant plastocyanin protein variant or fragment, thereby obtaining a plant that is cold-stress tolerant.
114. The method of paragraph 113, wherein the endonuclease is a Cas endonuclease;
   optionally, wherein the guide RNA is a clustered regularly interspaced short palindromic repeats (CRISPR)-associated (Cas)-guide RNA.
115. A seed produced by a plant of any one of paragraphs 105 to 107, wherein said seed comprises a detectable amount of the variant polynucleotide encoding a plastocyanin protein variant or fragment;
   optionally, wherein the seed comprises a transgene comprising a heterologous variant polynucleotide, or
   optionally, wherein the seed comprises an endogenous edited gene comprising the variant polynucleotide.
116. The seed of paragraph 115, wherein the seed is coated with a composition comprising an insecticide and/or a fungicide.
117. A plant propagation material comprising the coated seed of paragraph 116.
118. A method of producing/breeding a cold-stress tolerant plant, the method comprising crossing the cold-stress tolerant plant of any one of paragraphs 105 to 107 with one or more other plants to produce a population of progeny plants.
119. The method of paragraph 118, further comprising screening the population of progeny plants to identify cold-stress tolerant plants.
120. The method of paragraph 119, wherein the population of plants is screened by genotyping to detect a variant polynucleotide encoding for plastocyanin protein variant or fragment.
121. The method of any one of paragraphs 118 to 120, further comprising selecting a progeny plant based on its genotype and/or phenotype.
122. A plant of any one of paragraphs 105 to 107 or seed of paragraph 115 or 116 for use in a method of plant breeding, crop production, or for making a processed plant product.

### REFERENCES

1. Brenton, Z. W., Cooper, E. A., Myers, M. T., Boyles, R. E., Shakoor, N., Zielinski, K. J., Rauh, B. L., Bridges, W. C., Morris, G. P., Kresovich, S. (2016) A genomic resource for the development, improvement, and exploitation of sorghum for bioenergy. Genetics, 204(1): 21-33.
2. Yu, J., Tuinstra, M. R. (2001) Genetic analysis of seedling growth under cold temperature stress in grain sorghum. Crop Science, 41(5): 1438-1443.
3. Upadhyaya. H. D., Wang, Y. H., Sastry, D. V., Dwivedi, S. L., Prasad, P. V., Burrell, A. M., Morris, G. P., Klein, P. E. (2015) Association mapping of germinability and seedling vigor in sorghum under controlled low-temperature conditions. Genome, 59(2): 137-145.
4. Burow, G., Burke, J. J., Xin, Z., Franks. C. D. (2011) Genetic dissection of early-season cold tolerance in sorghum (Sorghum bicolor (L.) Moench). Molecular Breeding, 28(3): 391-402.
5. Salas Fernandez, M. G., Schoenbaum, G. R., Goggi, A. S. (2014) Novel germplasm and screening methods for early cold tolerance in Sorghum. Crop Science, 54(6): 2631-2638.
6. Chopra, R., Burow, G., Hayes, C., Emendack, Y., Xin, Z., Burke, J. (2015) Transcriptome profiling and validation of gene based single nucleotide polymorphisms (SNPs) in sorghum genotypes with contrasting responses to cold stress. BMC Genomics, 16(n): 1040.
7. Esechie, H. A., Maranville, J. W., Ross, W. M. (1977) Relationship of Stalk Morphology and Chemical Composition to Lodging Resistance in Sorghum 1. Crop Science, 17(4): 609-612.
8. Chopra, R., Burow, G., Burke, J. J., Gladman, N., Xin, Z. (2017) Genome-wide association analysis of seedling traits in diverse Sorghum germplasm under thermal stress. BMC Plant Biology, 17(1): 12.
9. Yu. J., Tuinstra, M. R., Claassen, M. M., Gordon, W. B., Witt, M. D. (2004) Analysis of cold tolerance in sorghum under controlled environment conditions. Field Crops Research, 85(1): 21-30.
10. Ortiz, D., Hu, J., Salas Fernandez, M. G. (2017) Genetic architecture of photosynthesis in Sorghum bicolor under non-stress and cold stress conditions. Journal of Experimental Botany, 68(16): 4545-4557.
11. Segura, V., Vilhjálmsson, B. J., Platt, A., Korte, A., Seren, Ü., Long, Q., Nordborg, M. (2012) An efficient multi-locus mixed-model approach for genome-wide association studies in structured populations. Nature Genetics, 44(7): 825.
12. Sigfridsson, K. (1998) Plastocyanin, an electron-transfer protein. Photosynthesis Research, 57(1): 1-28.
13. Makita, Y., Shimada, S., Kawashima, M., Kondou-Kuriyama, T., Toyoda, T., Matsui. M. (2014) MOROKOSHI: transcriptome database in Sorghum bicolor. Plant and Cell Physiology, 6(1): e6-e6.
14. Murphy, R. L., Klein, R R., Morishige, D. T., Brady, J. A., Rooney, W. L., Miller, F. R., Dugas, D. V., Klein, P. E., Mullet, J. E. (2011) Coincident light and clock regulation of pseudoresponse regulator protein 37 (PRR37) controls photoperiodic flowering in sorghum. Proceedings of the National Academy of Sciences, 108(39): 16469-16474.
15. Liu, X., Zhou. Y., Xiao, J., Bao, F. (2018) Effects of Chilling on the Structure. Function and Development of Chloroplasts. Frontiers in Plant Science, 9: 1715.
16. Zhang, J. P., Yu, Y., Feng, Y. Z., Zhou. Y. F., Zhang, F., Yang, Y. W., Lei, M. Q., Zhang, Y. C., Chen, Y. Q. (2017) miRNA MiR408 regulates grain yield and photosynthesis via a phytocyanin protein. Plant Physiology, 175: 1175-1185.
17. Fahlgren, N., Feldman, M., Gehan, M. A., Wilson, M. S., Shyu, C., Bryant, D. W., Hill, S. T., McEntee, C. J., Wamasooriya, S. N., Kumar, I., Ficor, T., Tumipseed. S., Gilbert, K. B., Brutnell, T. P., Carrington, J. C., Mockler, T. C., Baxter, 1. (2015) A versatile phenotyping system and analytics platform reveals diverse temporal responses to water availability in Setaria. Molecular Plant, 8(10): 1520-1535.
18. Hoffmann, W. A., & Poorter, H. (2002) Avoiding bias in calculations of relative growth rate. Annals of Botany, 90(1): 37-42.
19. Davies, L., Gather, U. (1993) The Identification of Multiple Outliers. Journal of the American Statistical Association, 88(423): 782-792.
20. Feldman, M. J., Paul, R. E., Banan, D., Barrett, J. F., Sebastian, J., Yee, M. C., Jiang, H., Lipka, A. E., Brutnell, T. P., Dinneny, J. R., Leakey, A. D. B., Baxter, I. (2017) Time dependent genetic analysis links field and controlled environment phenotypes in the model C4 grass Setaria. PLoS Genetics, 13(6): e1006841.
21. Bates, D., Mächler, M., Bolker, B., Walker, S. (2015) Fitting Linear Mixed-Effects Models Using lme4. Journal of Statistical Software., 67(1): 1-48.
22. Venables, W. N., Ripley, B. D. (2002) Random and mixed effects. Modern applied statistics with S Statistics and Computing, pp. 271-300.
23. Murtagh, F., Legendre, P. (2014) Ward's hierarchical agglomerative clustering method: which algorithms implement Ward's criterion? Journal of Classification, 31(3): 274-295.
24. Galili T (2010) Clustergram: visualization and diagnostics for cluster analysis. http://www.r-statistics.com/2010/06/clustergram-visualization-and-diagnostics-for-cluster-analysis-r-code/.
25. Schonlau, M. (2002) The clustergram: A graph for visualizing hierarchical and non-hierarchical cluster analyses. The Stata Journal, 3: 316-327.
26. Nakazawa, M., & Nakazawa, M. M. (2018) fmsb: Functions for Medical Statistics Book with some Demographic Data. R package version 0.6.3.
27. Kolde, R., & Kolde, M. R. (2018) pheatmap: Pretty Heatmaps. R package version 1.0.10.
28. Price, A. L., Patterson, N. J., Plenge, R. M., Weinblatt, M. E., Shadick, N. A., Reich, D. (2006) Principal components analysis corrects for stratification in genome-wide association studies. Nature Genetics, 38(8): 904.
29. Wimmer, V., Albrecht, T., Auinger, H. J., Schön, C. C. (2012) Synbreed: a framework for the analysis of genomic prediction data using R. Bioinformatics, 28(15): 2086-2087.
30. Astle, W., Balding, D. J. (2009) Population structure and cryptic relatedness in genetic association studies. Statistical Science, 24(4): 451-471.
31. Chen, J., Chen, Z. (2008) Extended Bayesian information criteria for model selection with large model spaces. Biometrika, 95(3): 759-771.
32. Ziegler, G. R., Hartsock, R. H., & Baxter, I. (2015) Zbrowse: an interactive GWAS results browser. PeerJ Computer Science, 1: e3.
33. Paterson, A. H., Bowers, J. E., Bruggmann, R., Dubchak, I., Grimwood, J., Gundlach. H., Haberer, G., Hellsten, U., Mitros, T., Poliakov, A., Schmutz, J. (2009) The Sorghum bicolor genome and the diversification of grasses. Nature, 457(7229): 551.
34. Mace, E. S., Tai, S., Gilding, E. K., Li, Y., Prentis, P. J., Bian, L., Campbell, B. C., Hu, W., Innes, D. J., Han, X., Cruickshank, A. (2013) Whole-genome sequencing reveals untapped genetic potential in Africa's indigenous cereal crop sorghum. Nature Communications, 4: 2320.
35. McCormick, R. F., Truong, S. K., Sreedasyam, A., Jenkins, J., Shu, S., Sims, D., Kennedy. M., Amirebrahimi, M., Weers, B. D., McKinley, B., Mattison, A. (2018) The Sorghum bicolor reference genome: improved assembly, gene annotations, a transcriptome atlas, and signatures of genome organization. The Plant Journal, 93(2): 338-354.
36. Danecek, P., Auton, A., Abecasis, G., Albers, C. A., Banks, E., DePristo, M. A., Handsaker, R., Lunter, G., Marth, G., Sherry, S. T., McVean, G., Durbin, R. (2011) The variant call format and VCFtools. Bioinformatics, 27(15): 2156-2158.
37. Cingolani, P., Platts, A., Wang, L. L., Coon, M., Nguyen, T., Wang, L., Land, S. J., Lu, X., Ruden, D. M. (2012) A program for annotating and predicting the effects of single nucleotide polymorphisms, SnpEff: SNPs in the genome of Drosophila melanogaster strain w1118; iso-2; iso-3. Fly, 6(2): 80-92.
38. Kissen, R., Eberl, F., Winge, P., Uleberg, E., Martinussen, I., Bones, A. M. (2016) Effect of growth temperature on glucosinolate profiles in Arabidopsis thaliana accessions. Phytochemistry, 130: 106-118.
39. Lee, B. H., Henderson, D. A., Zhu, J. K. (2005) The Arabidopsis cold-responsive transcriptome and its regulation by ICE1. The Plant Cell, 17(11): 3155-3175.
40. Liu, Z., Boachon, B., Lugan, R., Tavares, R., Erhardt, M., Mutterer, J., Demais, V., Pateyron, S., Brunaud, V., Ohnishi, T., Pencik, A., Achard, P., Gong, F., Hedden, P., Werck-Reichart, D., Renault, H. (2015) A conserved cytochrome P450 evolved in seed plants regulates flower maturation. Molecular Plant, 8(12): 1751-1765.
41. Carvallo, M. A., Pino, M. T., Jeknić, Z., Zou, C., Doherty, C. J., Shiu, S. H., Chen, T. H. H., Thomashow, M. F. (2011) A comparison of the low temperature transcriptomes and CBF regulons of three plant species that differ in freezing tolerance: Solanum commersonii, Solanum tuberosum, and Arabidopsis thaliana. Journal of Experimental Botany, 62(11): 3807-3819.
42. McCormick, A. J., Cramer, M. D., Watt, D. A. (2008) Differential expression of genes in the leaves of sugarcane in response to sugar accumulation. Topical Plant Biology, 1(2): 142-158.
43. Li, K., Wang, H., Hu, X., Liu, Z., Wu, Y., Huang, C. (2016) Genome-wide association study reveals the genetic basis of stalk cell wall components in maize. PloS One, 11(8): e0158906.
44. Vannini, C., Iriti, M., Bracale, M., Locatelli, F., Faoro, F., Croce, P., Pirona, R., Di Maro, A., Coraggio, I., Genga, A. (2006) The ectopic expression of the rice Osmyb4 gene in Arabidopsis increases tolerance to abiotic, environmental and biotic stresses. Physiological and Molecular Plant Pathology, 69(1): 26-42.
45. Hoe Kim, J., Tsukaya, H. (2015) Regulation of plant growth and development by the GROWTH-REGULATING FACTOR and GRF-INTERACTING FACTOR duo. Journal of experimental botany, 66(20): 6093-6107.
46. Horiguchi, G., Gonzalez, N., Beemster, G. T., Inzé, D., Tsukaya, H. (2009) Impact of segmental chromosomal duplications on leaf size in the grandifolia-D mutants of Arabidopsis thaliana. The Plant Journal, 60(1): 122-133.
47. Sharma, N., Cram, D., Huebert, T., Zhou, N., Parkin, I. A. (2007) Exploiting the wild crucifer Thlaspi arvense to identify conserved and novel genes expressed during a plant's response to cold stress. Plant Molecular Biology, 63(2): 171-184.
48. Ogundiwin, E. A., Marti, C., Forment, J., Pons, C., Granell, A., Gradziel, T. M., Peace, C. P., Crisosto, C. H. (2008) Development of ChillPeach genomic tools and identification of cold-responsive genes in peach fruit. Plant Molecular Biology, 68(4-5): 379.
49. Dametto, A., Sperotto, R. A., Adamski, J. M., Blasi, É. A., Cargnelutti, D., de Oliveira, L. F., Ricachenevsky, F. K., Fregonezi, J. N., Mariath, J. E. A., da Cruz, R. P., Margis, R., Fett, J. P. (2015) Cold tolerance in rice germinating seeds revealed by deep RNAseq analysis of contrasting indica genotypes. Plant Science, 238: 1-12.
50. Chan, Z., Wang, Y., Cao, M., Gong, Y., Mu, Z., Wang, H., Hu, Y., Deng, X., He, X. J., Zhu, J. K. (2016) RDM4 modulates cold stress resistance in Arabidopsis partially through the CBF-mediated pathway. New Phytologist, 209(4): 1527-1539.
51. Kim, B. H., Kim, S. Y., Nam, K. H. (2012) Genes encoding plant-specific class III peroxidases are responsible for increased cold tolerance of the brassinosteroid-insensitive 1 mutant. Molecules and Cells, 34(6): 539-548.
52. Yang, Y., Liao, W., Yu, X., Wang, B., Peng, M., Ruan, M. (2016) Overexpression of MeDREBID confers tolerance to both drought and cold stresses in transgenic Arabidopsis. Acta Physiologiae Plantarum, 38(10): 243.
53. Raggi, S., Ferrarini, A., Delledonne, M., Dunand, C., Ranocha, P., De Lorenzo, G. Cervone F., Ferrari, S. (2015) The Arabidopsis thaliana Class III Peroxidase AtPRX71 Negatively Regulates Growth under Physiological Conditions and in Response to Cell Wall Damage. Plant Physiology, pp-01464, DOI: 10.1104/pp. 15.01464.
54. Tittarelli, A., Santiago, M., Morales, A., Meisel, L. A., Silva, H. (2009) Isolation and functional characterization of cold-regulated promoters, by digitally identifying peach fruit cold-induced genes from a large EST dataset. BMC Plant Biology, 9(1): 121.
55. Hemsley, P. A., Hurst, C. H., Kaliyadasa, E., Lamb, R., Knight, M. R., De Cothi, E. A., Steele, J. F., Knight, H. (2014) The Arabidopsis mediator complex subunits MED16, MED14, and MED2 regulate mediator and RNA polymerase II recruitment to CBF-responsive cold-regulated genes. The Plant Cell, 26(1): 465-484.
56. Maruyama, K., Sakuma, Y., Kasuga, M., Ito, Y., Seki, M., Goda, H., Shimada, Y., Yoshida, S., Shinozaki, K., Yamaguchi-Shinozaki, K. (2004) Identification of cold-inducible downstream genes of the Arabidopsis DREB1A/CBF3 transcriptional factor using two microarray systems. The Plant Journal, 38(6): 982-993.
57. Oono, Y., Seki, M., Satou, M., Iida, K., Akiyama, K., Sakurai, T., Fujita, M., Yamaguchi-Shinozaki, K., Shinozaki, K. (2006) Monitoring expression profiles of Arabidopsis genes during cold acclimation and deacclimation using DNA microarrays. Functional & Integrative Genomics, 6(3): 212-234.
58. Baute, J., Polyn, S., De Block, J., Blomme, J., Van Lijsebettens, M., Inzé, D. (2017) F-Box Protein FBX92 Affects Leaf Size in Arabidopsis thaliana. Plant and Cell Physiology, 58(5): 962-975.
59. Zhang, Q., Cheetamun, R., Dhugga, K. S., Rafalski, J. A., Tingey, S. V., Shirley, N. J., Taylor, J., Hayes, K., Beatty, M., Bacic, A., Burton, R. A., Fincher, G. B. (2014) Spatial gradients in cell wall composition and transcriptional profiles along elongating maize internodes. BMC Plant Biology, 14(1): 27.
60. Teige, M., Scheikl, E., Eulgem, T., Dóczi, R., Ichimura, K., Shinozaki, K., Dangl, J. L., Hirt, H. (2004) The MKK2 pathway mediates cold and salt stress signaling in Arabidopsis. Molecular Cell, 15(1): 141-152.
61. Moon, S. J., Han, S. Y., Kim, D. Y., Kim, B. G., Yoon, I. S., Shin, D., Kwon, H. B., Byun, M. O. (2014) Ectopic expression of CaWRKYI, a pepper transcription factor, enhances drought tolerance in transgenic potato plants. Journal of Plant Biology, 57(3): 198-207.
62. Abdeen, A., Schnell, J., Miki, B. (2010) Transcriptome analysis reveals absence of unintended effects in drought-tolerant transgenic plants overexpressing the transcription factor ABF3. BMC Genomics, 11(1): 69.
63. Janská, A., Aprile, A., Zámečnik, J., Cattivelli, L., Ovesná, J. (2011) Transcriptional responses of winter barley to cold indicate nucleosome remodeling as a specific feature of crown tissues. Functional & Integrative Genomics, 11(2): 307-325.
64. Jeong, Y. J., Choy, Y. H., Joo, H. J., Hwang, J. H., Byun, Y. J., Ha-Lee, Y. M., Lee, J. S., Jang, Y. S., Lee, D. H. (2012) Identification and analysis of cold stress-inducible genes in Korean rapeseed varieties. Journal of Plant Biology, 55(6): 498-512.
65. Kim, M. H., Sato, S., Sasaki, K., Saburi, W., Matsui, H., Imai, R. (2013) COLD SHOCK DOMAIN PROTEIN 3 is involved in salt and drought stress tolerance in Arabidopsis. FEBS Open Bio, 3(1): 438-442.
66. Robinson, S. J., Parkin, I. A. (2008) Differential SAGE analysis in Arabidopsis uncovers increased transcriptome complexity in response to low temperature. BMC Genomics, 9(1): 434.
67. Delong Meng, X. Y., Ma, L., Hu, J., Liang, Y., Liu, X., Yin, H., Liu, H., He, X., Li, D. (2017) Transcriptomic response of Chinese yew (Taxus chinensis) to cold stress. Frontiers in Plant Science, 8: 468.
68. Shimizu, N., Ishida, T., Yamada, M., Shigenobu, S., Tabata, R., Kinoshita, A., Yamaguchi, K., Hasebe, M., Mitsumasu, K., Sawa, S. (2015) BAM 1 and RECEPTOR-LIKE PROTEIN KINASE 2 constitute a signaling pathway and modulate CLE peptide-triggered growth inhibition in Arabidopsis root. New Phytologist, 208(4): 1104-1113.
69. Zhao, Z., Tan, L., Dang, C., Zhang, H., Wu, Q., An, L. (2012) Deep-sequencing transcriptome analysis of chilling tolerance mechanisms of a subnival alpine plant, Chorispora bungeana. BMC Plant Biology, 12(1): 222.
70. Ou, Y., Lu, X., Zi, Q., Xun, Q., Zhang, J., Wu, Y., Shi, H., Wei, Z., Zhao, B., Zhang, X., He, K., Gou, X., Li, C., Li, J. (2016) RGF1 INSENSITIVE 1 to 5, a group of LRR receptor-like kinases, are essential for the perception of root meristem growth factor 1 in Arabidopsis thaliana. Cell Research, 26(6): 686-698.
71. Shinohara, H., Mori, A., Yasue, N., Sumida, K., Matsubayashi, Y. (2016) Identification of three LRR-RKs involved in perception of root meristem growth factor in Arabidopsis. Proceedings of the National Academy of Sciences, 113(14): 3897-3902.
72. Zhang, Z., Li, J., Pan, Y., Li, J., Shi, H., Zeng, Y., . . . & Sun, X. (2017). Natural variation in CTB4a enhances rice adaptation to cold habitats. Nature communications, 8, 14788.
73. Zhang, Z. F., Li, Y. Y., Xiao, B. Z. (2016) Comparative transcriptome analysis highlights the crucial roles of photosynthetic system in drought stress adaptation in upland rice. Scientfic Reports, 6: 19349.
74. Kim, M. H., Sonoda, Y., Sasaki, K., Kaminaka, H., Imai, R. (2013) Interactome analysis reveals versatile functions of Arabidopsis COLD SHOCK DOMAIN PROTEIN 3 in RNA processing within the nucleus and cytoplasm. Cell Stress and Chaperones, 18(4): 517-525.
75. Zhang, F., Zhou, Y., Zhang, M., Luo, X., Xie, J. (2017) Effects of drought stress on global gene expression profile in leaf and root samples of Dongxiang wild rice (Oryza rufipogon). Bioscience Reports, 37(3): BSR20160509.
76. Ray, S., Dansana, P. K., Giri, J., Deveshwar, P., Arora, R., Agarwal, P., Khurana, J. P., Kapoor, S., Tyagi, A. K. (2011) Modulation of transcription factor and metabolic pathway genes in response to water-deficit stress in rice. Functional & Integrative Genomics, 11(1): 157-178.
77. Barah, P., Jayavelu, N. D., Rasmussen, S., Nielsen, H. B., Mundy, J., Bones, A. M. (2013) Genome-scale cold stress response regulatory networks in ten Arabidopsis thaliana ecotypes. BMC Genomics, 14(1): 722.
78. Zheng, J., Xi, M., La, Y., Lu, Y., Shi, J. (2013) Transcriptional analysis provides new insights into cold- and dehydration-tolerance signaling pathways and on regulation of stem cell activity in the vascular cambium of poplar. Plant Molecular Biology Reporter, 31(1) 75-86.
79. Chinnusamy, V., Zhu, J., Zhu, J. K. (2006) Gene regulation during cold acclimation in plants. Physiologia Plantarum, 126(1): 52-61.
80. Catalá, R., López-Cobollo, R., Castellano, M. M., Angosto, T., Alonso, J. M., Ecker, J. R., Salinas, J. (2014) The Arabidopsis 14-3-3 protein RARE COLD INDUCIBLE 1A links low-temperature response and ethylene biosynthesis to regulate freezing tolerance and cold acclimation. The Plant Cell, 26(8): 3326-3342.
81. Bai, B., Wu, J., Sheng, W. T., Zhou, B., Zhou, L. J., Zhuang, W., Yao, D. P., Deng, Q. Y. (2015) Comparative analysis of anther transcriptome profiles of two different rice male sterile lines genotypes under cold stress. International Journal of Molecular Sciences, 16(5): 11398-11416.
82. Shan, X., Li, Y., Jiang, Y., Jiang, Z., Hao, W., Yuan, Y. (2013) Transcriptome profile analysis of maize seedlings in response to high-salinity, drought and cold stresses by deep sequencing. Plant Molecular Biology Reporter, 31(6): 1485-1491.
83. Xin, Z., Mandaokar, A., Chen, J., Last, R. L., Browse, J. (2007) Arabidopsis ESK1 encodes a novel regulator of freezing tolerance. The Plant Journal, 49(5): 786-799.
84. Lee, J. Y., Joo, H., Choy, Y. H., Ha-Lee, Y. M., Lee, D. H. (2010) Using specialized cDNA microarrays to analyze Arabidopsis gene expression under cold stress. Journal of Plant Biology, 53(3): 240-250.
85. Bouchabke-Coussa, O., Quashie, M. L., Seoane-Redondo, J., Fortabat, M. N., Gery, C., Yu, A., Linderme, D., Trouverie, J., Granier, F., Teoule, E., Durand-Tardif, M. (2008) ESKIMO1 is a key gene involved in water economy as well as cold acclimation and salt tolerance. BMC Plant Biology, 8(1): 125.
86. Chen, H., Chen, X., Chen, D., Li, J., Zhang, Y., Wang, A. (2015) A comparison of the low temperature transcriptomes of two tomato genotypes that differ in freezing tolerance: Solanum lycopersicum and Solanum habrochaites. BMC Plant Biology, 15(1): 132.
87. Hu, H., You, J., Fang, Y., Zhu, X., Qi, Z., Xiong, L. (2008) Characterization of transcription factor gene SNAC2 conferring cold and salt tolerance in rice. Plant Molecular Biology, 67(1-2): 169-181.
88. Bosch, M., Mayer, C. D., Cookson, A., Donnison, I. S. (2011) Identification of genes involved in cell wall biogenesis in grasses by differential gene expression profiling of elongating and non-elongating maize internodes. Journal of Experimental Botany, 62(10): 3545-3561.
89. Shi, Y., Tian, S., Hou, L., Huang, X., Zhang, X., Guo, H., Yang, S. (2012) Ethylene signaling negatively regulates freezing tolerance by repressing expression of CBF and type-A ARR genes in Arabidopsis. The Plant Cell, 24(6): 2578-2595.
90. Kim, J., Wilson, R. L., Case, J. B., Binder, B. M. (2012) A comparative study of ethylene growth response kinetics in eudicots and monocots reveals a role for gibberellin in growth inhibition and recovery. Plant Physiology, 160(3): 1567-1580.
91. Potuschak, T., Lechner, E., Parmentier, Y., Yanagisawa, S., Grava, S., Koncz, C., Genschik, P. (2003) EIN3-dependent regulation of plant ethylene hormone signaling by two Arabidopsis F box proteins: EBF1 and EBF2. Cell, 115(6): 679-689.
92. Gagne, J. M., Smalle, J., Gingerich, D. J., Walker, J. M., Yoo, S. D., Yanagisawa, S., Vierstra, R. D. (2004) Arabidopsis EIN3-binding F-box 1 and 2 form ubiquitin-protein ligases that repress ethylene action and promote growth by directing EIN3 degradation. Proceedings of the National Academy of Sciences of the United States of America, 101(17): 6803-6808.
93. Schwager, K. M., Calderon-Villalobos, L. I. A., Dohmann, E. M., Willige, B. C., Knierer, S., Nill, C., Schwechheimer, C. (2007) Characterization of the VIER F-BOX PROTEINE genes from Arabidopsis reveals their importance for plant growth and development. The Plant Cell, 19(4): 1163-1178.
94. Lundmark, M., Cavaco, A. M., Trevanion, S., Hurry, V. (2006) Carbon partitioning and export in transgenic Arabidopsis thaliana with altered capacity for sucrose synthesis grown at low temperature: a role for metabolite transporters. Plant, Cell & Environment, 29(9): 1703-1714.
95. Lv, Y., Yang, M., Hu, D., Yang, Z., Ma, S., Li, X., Xiong, L. (2017) The OsMYB30 transcription factor suppresses cold tolerance by interacting with a JAZ protein and suppressing β-amylase expression. Plant Physiology, 173(2): 1475-1491.
96. Gille, S., Sharma, V., Baidoo, E. E., Keasling, J. D., Scheller, H. V., Pauly, M. (2013) Arabinosylation of a Yariv-precipitable cell wall polymer impacts plant growth as exemplified by the Arabidopsis glycosyltransferase mutant ray1. Molecular Plant, 6(4): 1369-1372.
97. Bonnecarrere, V., Quero, G., Monteverde, E., Rosas, J., De Vida, F. P., Cruz, M., Corredor, E., Garaycochea, S., Monza, J., Borsani, O. (2015) Candidate gene markers associated with cold tolerance in vegetative stage of rice (Oryzasativa L.). Euphytica, 203(2): 385-398.
98. Muraya, M. M., Schmutzer, T., Ulpinnis, C., Scholz, U., Altmann, T. (2015) Targeted sequencing reveals large-scale sequence polymorphism in maize candidate genes for biomass production and composition. PLoS One, 10(7): e0132120.
99. Wang, X., Wang, H., Long, Y., Liu, L., Zhao, Y., Tian, J. Zhao, W., Li, B., Li, C., Chao, H., Li, M. (2015) Dynamic and comparative QTL analysis for plant height in different developmental stages of Brassica napus L. Theoretical and Applied Genetics, 128(6): 1175-1192.
100. Alcázar, R., García-Martínez, J. L., Cuevas, J. C., Tiburcio, A. F., Altabella, T. (2005) Overexpression of ADC2 in Arabidopsis induces dwarfism and late-flowering through GA deficiency. The Plant Journal, 43(3): 425-436.
101. Kotoda, N., Matsuo, S., Honda, I., Yano, K., Shimizu, T. (2017) Gibberellin 2-Oxidase Genes from Satsuma Mandarin (Citrus unshiu Marc.) Caused Late Flowering and Dwarfism in Transgenic Arabidopsis. The Horticulture Journal, 86(2): 183-193.
102. Provart, N. J., Gil, P., Chen, W., Han, B., Chang, H. S., Wang, X., Zhu, T. (2003) Gene expression phenotypes of Arabidopsis associated with sensitivity to low temperatures. Plant Physiology, 132(2): 893-906.
103. Zhang, B., Sun, C., Xie, D., Tan, L. (2014) Global gene expression analysis of a rice high-tillering dwarf mutant. Genes & Genomics, 36(4): 485-496.
104. Yang, Y. W., Chen, H. C., Jen, W. F., Liu, L. Y., Chang, M. C. (2015) Comparative transcriptome analysis of shoots and roots of TNG67 and TCN1 rice seedlings under cold stress and following subsequent recovery: Insights into metabolic pathways, phytohormones, and transcription Factors. PloS One, 10(7): e0131391.
105. Mao, J., Yu, Y., Yang, J., Li, G., Li, C., Qi, X., Wen, T., Hu, J. (2017) Comparative transcriptome analysis of sweet corn seedlings under low-temperature stress. The Crop Journal, 5(5): 396-406.
106. Rui, Y., Xiao, C., Yi, H., Kandemir, B., Wang, J. Z., Puri, V. M., Anderson, C. T. (2017) POLYGALACTURONASE INVOLVED IN EXPANSION3 Functions in Seedling Development, Rosette Growth, and Stomatal Dynamics in Arabidopsis thaliana. The Plant Cell Online, tpc-00568.
107. Dong, C. H., Hu, X., Tang, W., Zheng, X., Kim, Y. S., Lee, B. H., Zhu, J. K. (2006) A putative Arabidopsis nucleoporin, AtNUP160, is critical for RNA export and required for plant tolerance to cold stress. Molecular and Cellular Biology, 26(24): 9533-9543.
108. Xiao, C., Somerville, C., Anderson, C. T. (2014) POLYGALACTURONASE INVOLVED IN EXPANSION1 functions in cell elongation and flower development in Arabidopsis. The Plant Cell, 26(3): 1018-1035.
109. Chen, M., Markham, J. E., Dietrich, C. R., Jaworski, J. G., Cahoon, E. B. (2008) Sphingolipid long-chain base hydroxylation is important for growth and regulation of sphingolipid content and composition in Arabidopsis. The Plant Cell, 20(7): 1862-1878.
110. Hwang, I., Manoharan, R. K., Kang, J. G., Chung, M. Y., Kim, Y. W., Nou, I. S. (2016) Genome-Wide Identification and Characterization of bZIP Transcription Factors in Brassica oleracea under Cold Stress. BioMed Research International, 2016:4376598.
111. Huo, C., Zhang, B., Wang, H., Wang, F., Liu, M., Gao, Y., Zhang, W., Deng, Z., Sun, D., Tang, W. (2016) Comparative study of early cold-regulated proteins by two-dimensional difference gel electrophoresis reveals a key role for phospholipase Dal in mediating cold acclimation signaling pathway in rice. Molecular & Cellular Proteomics, 15(4): 1397-1411.
112. Sasaki, Y., Takahashi, K., Oono, Y., Seki, M., Yoshida, R., Shinozaki, K., Uemura, M. (2008) Characterization of growth-phase-specific responses to cold in Arabidopsis thaliana suspension-cultured cells. Plant, Cell & Environment, 31(3): 354-365.
113. Xiao, S., Chye, M. L. (2011) Overexpression of Arabidopsis ACBP3 enhances NPR1-dependent plant resistance to Pseudomonas syringe pv tomato DC3000. Plant Physiology, 156(4): 2069-2081.
114. Nair, P., Kandasamy, S., Zhang, J., Ji, X., Kirby, C., Benkel, B., Hodges, M. D., Critchley, A. T., Hiltz, D., Prithiviraj, B. (2012) Transcriptional and metabolomic analysis of Ascophyllum nodosum mediated freezing tolerance in Arabidopsis thaliana. BMC Genomics, 13(1): 643.
115. Gasperini, D., Greenland, A., Hedden, P., Dreos, R., Harwood, W., Griffiths, S. (2012) Genetic and physiological analysis of Rht8 in bread wheat: an alternative source of semi-dwarfism with a reduced sensitivity to brassinosteroids. Journal of Experimental Botany, 63(12): 4419-4436.
116. Byun, M. Y., Lee, J., Cui, L. H., Kang, Y., Oh, T. K., Park, H., Lee, H., Kim, W. T. (2015) Constitutive expression of DaCBF7, an Antarctic vascular plant Deschampsia antarctica CBF homolog, resulted in improved cold tolerance in transgenic rice plants. Plant Science, 236: 61-74.
117. Fan, M., Wang, M., Bai, M. Y. (2016) Diverse roles of SERK family genes in plant growth, development and defense response. Science China Life Sciences, 59(9): 889-896.
118. Jiang, L., Ye, M., Zhu, S., Zhai, Y., Xu, M., Huang, M., Wu, R. (2016) Computational identification of genes modulating stem height-diameter allometry. Plant Biotechnology Journal, 14(12): 2254-2264.
119. Ko, J. H., Han, K. H. (2004). Arabidopsis whole-transcriptome profiling defines the features of coordinated regulations that occur during secondary growth. Plant Molecular Biology, 55(3): 433-453.
120. Zhao, Z., Assmann, S. M. (2011) The glycolytic enzyme, phosphoglycerate mutase, has critical roles in stomatal movement, vegetative growth, and pollen production in Arabidopsis thaliana. Journal of Experimental Botany, 62(14): 5179-5189.
121. Cho, H. Y., Hwang, S. G., Kim, D. S., Jang, C. S. (2012) Genome-wide transcriptome analysis of rice genes responsive to chilling stress. Canadian Journal of Plant Science, 92(3): 447-460.
122. Ye, W., Shen, C. H., Lin, Y., Chen, P. J., Xu, X., Oelmüller, R., Yeh, K., Lai, Z. (2014) Growth promotion-related miRNAs in Oncidium orchid roots colonized by the endophytic fungus Piriformospora indica. PLoS One, 9(1): e84920.
123. Liu, Q., Hu, H., Zhu, L., Li, R., Feng, Y., Zhang, L., Yang., Y., Liu, X., Zhang, H. (2015) Involvement of miR528 in the regulation of arsenite tolerance in rice (Oryza sativa L.). Journal of Agricultural and Food Chemistry, 63(40): 8849-8861.
124. Zhang, H., Li, L. (2013) SQUAMOSA promoter binding protein-like7 regulated microRNA408 is required for vegetative development in Arabidopsis. The Plant Journal, 74(1): 98-109.
125. Ohashi-Ito, K, Oda, Y., Fukuda, H. (2010) Arabidopsis VASCULAR-RELATED NAC-DOMAIN6 directly regulates the genes that govern programmed cell death and secondary wall formation during xylem differentiation. The Plant Cell, 22(10): 3461-3473.
126. Kim, S., Kim, S. J., Shin, Y. J., Kang, J. H., Kim, M. R., Nam, K. H., Lee, M. S., Lee, S. H., Kim, Y. H., Hong, S. K., Verma, D. P. S., Chun, J. Y., Cheon, C. I. (2009) An atypical soybean leucine-rich repeat receptor-like kinase, GmLRK1, may be involved in the regulation of cell elongation. Planta, 229(4): 811-821.
127. Li, J., Liu, H., Xia, W., Mu, J., Feng, Y., Liu, R., Yan, P., Wang, A., Lin, Z., Guo, Y., Zhu, J., Zhu, J., Chen, X. (2017) De Novo Transcriptome Sequencing and the Hypothetical Cold Response Mode of Saussurea involucrata in Extreme Cold Environments. International Journal of Molecular Sciences, 18(6): 1155.
128. Maeda, S., Sakazono, S., Masuko-Suzuki, H., Taguchi, M., Yamamura, K, Nagano, K, Endo, T., Saeki, K., Osaka, M., Nabemoto, M., Ito, K, Kudo, T., Kobayashi, M., Kawagishi, M., Fujita, K, Nanjo, H., Shindo, T., Yano, K, Suzuki, G., Suwabe, K, Watanabe, W. (2016) Comparative analysis of microRNA profiles of rice anthers between cool-sensitive and cool-tolerant cultivars under cool-temperature stress. Genes & Genetic Systems, 91(2): 97-109.
129. Usadel, B., Blaesing, O. E., Gibon, Y., Poree, F., Hoehne, M., Guenter, M., Trethewey, R., Kamlafe, B., Poorter, H., Stitt, M. (2008) Multilevel genomic analysis of the response of transcripts, enzyme activities and metabolites in Arabidopsis rosettes to a progressive decrease of temperature in the non-freezing range. Plant, Cell & Environment, 31(4): 518-547.
130. Noir, S., Marrocco, K, Masoud, K, Thomann, A., Gusti, A., Bitrian, M., Schnittger, A., Genschik, P. (2015) The control of Arabidopsis thaliana growth by cell proliferation and endoreplication requires the F-box protein FBL17. The Plant Cell, 27(5): 1461-1476.
131. Rinaldi, M. A., Liu, J., Enders, T. A., Bartel, B., Strader, L. C. (2012) A gain-of-function mutation in IAA16 confers reduced responses to auxin and abscisic acid and impedes plant growth and fertility. Plant Molecular Biology, 79(4-5): 359-373.
132. An, D., Yang, J., Zhang, P. (2012) Transcriptome profiling of low temperature-treated cassava apical shoots showed dynamic responses of tropical plant to cold stress. BMC Genomics, 13(1): 64.
133. Chapman, E. J., Greenham, K, Castillejo, C., Sartor, R., Bialy, A., Sun, T. P., Estelle, M. (2012) Hypocotyl transcriptome reveals auxin regulation of growth-promoting genes through GA-dependent and -independent pathways. PLoS One, 7(5): e36210.
134. Grienenberger, E., Douglas, C. J. (2014). Arabidopsis VASCULAR-RELATED UNKNOWN PROTEIN1 Regulates Xylem Development and Growth by a Conserved Mechanism That Modulates Hormone Signaling. Plant Physiology, 164(4): 1991-2010.
135. PARK, M. R., YUN, K. Y., Mohanty, B., Herath, V., Xu, F., Wijaya, E., Bajic, V. B., Yun, S. J., De Los Reyes, B. G. (2010) Supra-optimal expression of the cold-regulated OsMyb4 transcription factor in transgenic rice changes the complexity of transcriptional network with major effects on stress tolerance and panicle development. Plant, Cell & Environment, 33(12): 2209-2230.
136. Willige, B. C., Ghosh, S., Nill, C., Zourelidou, M., Dohmann, E. M., Maier, A., Schwechheimer, C. (2007) The DELLA domain of GA INSENSITIVE mediates the interaction with the GA INSENSITIVE DWARF1A gibberellin receptor of Arabidopsis. The Plant Cell, 19(4): 1209-1220.
137. Achard, P., Gong, F., Cheminant, S., Alioua, M., Hedden, P., Genschik, P. (2008) The cold-inducible CBF1 factor-dependent signaling pathway modulates the accumulation of the growth-repressing DELLA proteins via its effect on gibberellin metabolism. The Plant Cell, 20(8): 2117-2129.
138. Zhou, M., Chen, H., Wei, D., Ma, H., Lin, J. (2017) Arabidopsis CBF3 and DELLAs positively regulate each other in response to low temperature. Scientific Reports, 7(2017): 39819.
139. Li, X., Yang, D., Sun, L., Li, Q., Mao, B., He, Z. (2016) The systemic acquired resistance regulator OsNPR1 Attenuates Growth by Repressing Auxin Signaling and promoting IAA-amido synthase expression. Plant Physiology, (2016): pp-00129.
140. Huda, K. M. K., Banu, M. S. A., Yadav, S., Sahoo, R. K., Tuteja, R., Tuteja, N. (2014) Salinity and drought tolerant OsACA6 enhances cold tolerance in transgenic tobacco by interacting with stress-inducible proteins. Plant Physiology and Biochemistry, 82: 229-238.
141. Zhang, G., Wang, F., Li, J., Ding, Q., Zhang, Y., Li, H., Zhang, J., Gao, J. (2015) Genome-wide identification and analysis of the VQ motif-containing protein family in Chinese cabbage (Brassica rapa L. ssp. Pekinensis). International Journal of Molecular Sciences, 16(12): 28683-28704.
142. Jeon, J., Kim, J. (2012). Arabidopsis response regulator 1 (ARR1) and Arabidopsis histidine phosphotransfer protein 2 (AHP2), AHP3, and AHP5 function in cold signaling. Plant Physiology, (2012): pp-112.
143. Shaik, R., Ramakrishna, W. (2013) Genes and co-expression modules common to drought and bacterial stress responses in Arabidopsis and rice. PloS One, 8(10): e77261.
144. Liu, Y., Zhou, M., Gao, Z., Ren, W., Yang, F., He, H., Zhao, J. (2015) RNA-seq analysis reveals MAPKKK family members related to drought tolerance in Maize. PloS One, 10(11): e0143128.
145. Cominelli, E., Galbiati, M., Vavasseur, A., Conti, L., Sala, T., Vuylsteke, M., Leonhardt, N., Dellaporta, S., Tonelli, C. (2005) A guard-cell-specific MYB transcription factor regulates stomatal movements and plant drought tolerance. Current Biology, 15(13): 1196-1200.
146. Tang, S., Liang, H., Yan, D., Zhao, Y., Han, X., Carlson, J. E., Xia, X., Yin, W. (2013) Populus euphratica the transcriptomic response to drought stress. Plant Molecular Biology, 83(6): 539-557.
147. Liu, F., Xu, W., Song, Q., Tan, L., Liu, J., Zhu, Z., Fu, Y., Sun, C. (2013) Microarray-assisted fine-mapping of quantitative trait loci for cold tolerance in rice. Molecular Plant, 6(3): 757-767.
148. Janská, A., Aprile, A., Cattivelli, L., Zámečnik, J., de Bellis, L., Ovesná, J. (2014) The up-regulation of elongation factors in the barley leaf and the down-regulation of nucleosome assembly genes in the crown are both associated with the expression of frost tolerance. Functional & Integrative Genomics, 14(3): 493-506.
149. Ding, B., Zhu, Y., Bu, Z. Y., Shen, W. H., Yu, Y., Dong, A. W. (2010) SDG714 regulates specific gene expression and consequently affects plant growth via H3K9 dimethylation. Journal of Integrative Plant Biology, 52(4): 420-430.
150. Tillett, R. L., Wheatley, M. D., Tattersall, E. A., Schlauch, K. A., Cramer, G. R., Cushman, J. C. (2012) The Vitis vinifera C-repeat binding protein 4 (VvCBF4) transcriptional factor enhances freezing tolerance in wine grape. Plant Biotechnology Journal, 10(1): 105-124.
151. Zhu, J., Zwiewka, M., Sovero, V., di Donato, M., Ge, P., Oehri, J., Aryal, B., Hao, P., Linnert, M., Burgardt, N., Lücke, C. (2016) TWISTED DWARF1 mediates the action of auxin transport inhibitors on actin cytoskeleton dynamics. The Plant Cell Online, tpc-00726.
152. Su, C. F., Wang, Y. C., Hsieh, T. H., Lu, C. A., Tseng, T. H., Yu, S. M. (2010) A novel MYBS3-dependent pathway confers cold tolerance in rice. Plant Physiology, 153(1): 145-158.

1-88. (canceled)

89. An edited plastocyanin gene comprising:
(i) a variant polynucleotide encoding a plastocyanin protein variant or fragment thereof,
wherein the variant polynucleotide comprises at least one nucleotide insertion, deletion, and/or substitution in comparison to the corresponding unedited wild-type polynucleotide sequence, and
wherein the variant polynucleotide does not encode a wild-type plastocyanin protein,
optionally, wherein said variant polynucleotide is operably linked to a polynucleotide comprising a promoter;
(ii) a variant polynucleotide comprising a plastocyanin gene 3′ UTR,
wherein the variant polynucleotide comprises at least one nucleotide insertion, deletion, and/or substitution in the 3′ UTR in comparison to the corresponding unedited wild-type polynucleotide sequence,
optionally wherein said variant polynucleotide is operably linked to a polynucleotide comprising a promoter and/or a plastocyanin protein coding region;
(iii) a variant polynucleotide comprising a plastocyanin gene 5′ UTR,
wherein the variant polynucleotide comprises at least one nucleotide insertion, deletion, and/or substitution in the 5′ UTR in comparison to the corresponding unedited wild-type polynucleotide sequence,
optionally wherein said variant polynucleotide is operably linked to a polynucleotide comprising a promoter and/or a plastocyanin protein coding region;
(iv) a variant polynucleotide comprising a plastocyanin gene promoter,
wherein the variant polynucleotide comprises at least one nucleotide insertion, deletion, and/or substitution in the promoter in comparison to the corresponding unedited wild-type polynucleotide sequence,
optionally, wherein said variant polynucleotide is operably linked to a polynucleotide comprising a plastocyanin protein coding region;
(v) a variant polynucleotide comprising a plastocyanin gene intron,
wherein the variant polynucleotide comprises at least one nucleotide insertion, deletion, and/or substitution in the intron in comparison to the corresponding unedited wild-type polynucleotide sequence,
optionally, wherein said variant polynucleotide is operably linked to a polynucleotide comprising at least one plastocyanin gene exon; and/or
(vi) a variant polynucleotide encoding a plastocyanin (a) transit peptide, vacuolar targeting peptide, and/or endoplasmic reticulum targeting peptide; (b) plastid targeting peptide; and/or (c) polyadenylation or transcriptional termination signal,
wherein the variant polynucleotide comprises at least one nucleotide insertion, deletion, and/or substitution in (a), (b), and/or (c) in comparison to the corresponding unedited wild-type polynucleotide sequence,
optionally, wherein the polynucleotides of (a), (b), and/or (c) are operably linked to a polynucleotide encoding a plastocyanin protein.

90. The edited plastocyanin gene of claim 89 comprising (i) variant polynucleotide encoding a plastocyanin protein variant or fragment thereof,
wherein the variant polynucleotide comprises at least one nucleotide insertion, deletion, and/or substitution in comparison to the corresponding unedited wild-type polynucleotide sequence,
wherein the variant polynucleotide does not encode a wild-type plastocyanin protein, and
wherein the variant polynucleotide encodes a plastocyanin protein variant or fragment thereof having reduced activity in comparison to the corresponding wild-type plastocyanin protein.

91. (canceled)

92. The edited plastocyanin gene of claim 90, wherein the nucleotide insertion, deletion, and/or substitution is in an exon of the edited plastocyanin gene;
optionally, wherein the nucleotide insertion, deletion, and/or substitution is in Exon 1 of the edited plastocyanin gene;
optionally, wherein the variant polynucleotide encodes a plastocyanin protein variant or fragment thereof comprising the amino acid sequence of SEQ ID NO: 8 or SEQ ID NO: 10;
optionally, wherein the variant polynucleotide comprises the nucleic acid sequence of SEQ ID NO: 7 or SEQ ID NO: 9.

93. (canceled)

94. The edited plastocyanin gene of claim 89 comprising (ii) a variant polynucleotide comprising a plastocyanin gene 3′ UTR,
wherein the variant polynucleotide comprises at least one nucleotide insertion, deletion, and/or substitution in the 3′ UTR in comparison to the corresponding unedited wild-type polynucleotide sequence; and
wherein the insertion, deletion, and/or substitution in the 3′ UTR results in reduced plastocyanin activity in comparison to an otherwise identical plastocyanin gene having a wild-type 3′ UTR;
optionally, wherein the variant polynucleotide comprising a plastocyanin gene 3′ UTR comprises SEQ ID NO: 11.

95. (canceled)

96. The edited plastocyanin gene of claim 89, wherein the variant polynucleotide is integrated into the nuclear or plastid genome of a cell.

97. A plant nuclear or plastid genome comprising the edited plastocyanin gene of claim 89.

98-101. (canceled)

102. A cell comprising the edited plastocyanin gene of claim 89.

103-104. (canceled)

105. A plant comprising the edited plastocyanin gene of claim 89.

106. The plant of claim 105, wherein the edited plastocyanin gene confers to the plant cold-stress tolerance in comparison to a control plant that lacks the edited gene.

107. The plant of claim 105, wherein plant is a monocot crop plant is selected from the group consisting of a corn, barley, oat, pearl millet, rice, sorghum, sugarcane, turf grass, and wheat plant.

108-114. (canceled)

115. A seed produced by a plant of claim 105, wherein said seed comprises a detectable amount of the variant polynucleotide.

116-117. (canceled)

118. A method of producing/breeding a cold-stress tolerant plant, the method comprising crossing the cold-stress tolerant plant of claim 105 with one or more other plants to produce a population of progeny plants.

119. The method of claim 118, further comprising screening the population of progeny plants to identify cold-stress tolerant plants.

120. The method of claim 119, wherein the population of plants is screened by genotyping to detect a variant polynucleotide encoding for plastocyanin protein variant or fragment.

121. The method of claim 118, further comprising selecting a progeny plant based on its genotype and/or phenotype.

122. (canceled)

123. A sorghum plant comprising the edited plastocyanin gene of claim 89.

124. The sorghum plant of claim 123, wherein the edited plastocyanin gene confers to the plant cold-stress tolerance in comparison to a control plant that lacks the edited gene.

125. A sorghum seed produced by a plant of claim 123, wherein said seed comprises a detectable amount of the variant polynucleotide.

126. A method of producing/breeding a cold-stress tolerant sorghum plant, the method comprising crossing the cold-stress tolerant sorghum plant of claim 123 with one or more other sorghum plants to produce a population of progeny sorghum plants.

127. The method of claim 126, further comprising screening the population of progeny sorghum plants to identify cold-stress tolerant plants.


SEQUENCE LISTING (22 sequences)

SEQ ID NO: 1 - DNA, 1704 bp, Sorghum bicolor
  5′UTR (1)..(157)
  Exon1 (158)..(344)
  Intron (345)..(895)
  Exon2 (896)..(1224)
  3′UTR (1225)..(1704)
acacgtatatatgtgaacgagttttctggctgcgcagcagctgggtatatacgtgcaggcatatatatagatgatcccactactccagtatacttcgaccctctctagctgacccagcctctcattaatttgcctcagtagtcttacgtgtatatatatggttgttcgagttcctatggctatggcggcggcggtactgctcgtggtggccgccgtcgtggtggtggtggaggtggccgcggcggcgacgtacacggtgggcgcgcccgccgggctgtgggacatgcagaccgactacgccgactgggtgaagagcaagacctttcaccccggcgacagcatcagttagtgatcacctctcacctctgtctctgtgtgagcacgcatgaaagttttttttattctaaagagcatatgctcccacttctttcatcactgaatttatattaaaaaatattccctccgtccctaaatactactatttctaggagaaaattttgtccacaaatactgctatttctactagcatactcagtccaccagcccatttaattctcctcggaacttccgtggtccaccagcctatttaatttcttctagggagtagtactttggcgcatgataattgtttgagtctgtttagttggcattaaatgcagctcattggaaccagagaaccatggcttaacgcgtatgattaaatggtggaacctaaattaattgagggtagaatggtcttttgtttgccatactaatctgtctgaaatttgctagaaatagcagtatttggggacggagggagtatataaacacatatttcaatatagatctcaattaaaaagtatgcaaatatatatctcaatacagattatacaatggtcctgtgtcctggcagcgttcacgtactcgccggagctgcacgacgtggtggaggtgatcaaggcgggctacgacgcctgctccagcgccaacaacatctccgccttccgctccggcaacgacgtcgtgacgctcaccgccgtcggcacgcgctacttcctctgcggcctcacgggacactgcggcaacggcatgaagatccgcgtcgacgtcgtcgccgccaaagcctcgtccgcgccggcgtcgagctcggccagcagggctgccgccgctggtttcgccatcatgacaaccgtcgccgcctcgctgctgctgctgcaacccgccgccgtcgtcgtcagctagcttcaccacgcgtgcgtggttgtgcgtttgctccatgtacactttgttattttcgcgcatgctagtgttttttttaatctcctctcagagcaagtattatagtaggctataagccagctaaatctgaggtggaggagagagaagaggagagagagtaaaagcggcctgtaagcttacagcggctcagacacaaaaaccaaaaaaatctatgagacagacaagtgggccatgtattaatggtgacgagctaaccattatatgagtaggctgcaagaaggctgtaaaaaaatcttacagccaacagctgactgtattataaaacttgctctcaacaagcaaaggacagcagcgcgtcaatgcattatgaatggccagatgggccgggtccaatgagcagcatggggcagggtgcttaggtacaacagcttaatgagctgttatgagttgttggttttttttctaaagactcttttacaaaac

SEQ ID NO: 2 - DNA, 1072 bp, Setaria viridis
  5′UTR (1)..(269)
  Exon1 (270)..(453)
  Intron (454)..(552)
  Exon2 (553)..(899)
  3′UTR (900)..(1072)
tgctcggtccagagcgctctctctccctctccttctcttaattaatactgcaattacccatctgtcgacgacgcggatcagcgataaaatccggcattaaacccacgccaaacgatcattgatccatgtgtccgtaggccgatttgatccatccactgtcattagggagtgtgtgaaccatggactaacaagtcctagactaactttatatatacatgcagatgtagagccagtgaaccagttcattgactgtcctccatcatttttatatggggacgagggctcttgttcctgcggcggcgatggcggcgcttgtggcggtgtccgccgtgatggaggcggcggcggcggcgacgacgtacacggtgggcgcgccggacgggctgtgggacatgcagaccgactacgcccagtgggtcaagaccaggaccttccaccccggcgacaagatcagtgagcttctcttcttttttttttccttctttttgatcctggtgtacgtggatcttctgtatgtactgtctgatcatatcctctgggacgacgttgcagtgttcacgtactcgccggagctgcacgacgtggtggaggtgaccaaggcggggtacgacgcctgctccagcgccaacaacatctccgccttccgctccggcaacgacgtcgtcgccctcgccgacgtcggcacccgctacttcctctgcggcctcacgggccactgcgacagcggcatgaagatccggatcgacgtcgtcgccgccgccgcgcctggcccagccgccgccgccgccgggccgtcctcctcctccgcgtccagggccgcggcggcctcttccgccgggatcgccgccgcctgcttgctcctgcagcacgccgtcgtcatcgcgcggctgctttggtagccatacccatagcttgtttgttagacgcgtcttaatattgtgcttagacgtttggtgttgctatacattacgtacttaattgtgtacgtagttttgttttagcctgtacacttttgttatttcaataccttggggtacttataataatactgttattcgtttattcaacgtgc

SEQ ID NO: 3 - DNA, 184 bp, Setaria viridis
atggggacgagggctcttgttcctgcggcggcgatggcggcgcttgtggcggtgtccgccgtgatggaggcggcggcggcggcgacgacgtacacggtgggcgcgccggacgggctgtgggacatgcagaccgactacgcccagtgggtcaagaccaggaccttccaccccggcgacaagatca

SEQ ID NO: 4 - DNA, 173 bp, Setaria viridis
ccatacccatagcttgtttgttagacgcgtcttaatattgtgcttagacgtttggtgttgctatacattacgtacttaattgtgtacgtagttttgttttagcctgtacacttttgttatttcaataccttggggtacttataataatactgttattcgtttattcaacgtgc

SEQ ID NO: 5 - DNA, 344 bp, Sorghum bicolor
acacgtatatatgtgaacgagttttctggctgcgcagcagctgggtatatacgtgcaggcatatatatagatgatcccactactccagtatacttcgaccctctctagctgacccagcctctcattaatttgcctcagtagtcttacgtgtatatatatggttgttcgagttcctatggctatggcggcggcggtactgctcgtggtggccgccgtcgtggtggtggtggaggtggccgcggcggcgacgtacacggtgggcgcgcccgccgggctgtgggacatgcagaccgactacgccgactgggtgaagagcaagacctttcaccccggcgacagcatca

SEQ ID NO: 6 - DNA, 480 bp, Sorghum bicolor
cttcaccacgcgtgcgtggttgtgcgtttgctccatgtacactttgttattttcgcgcatgctagtgttttttttaatctcctctcagagcaagtattatagtaggctataagccagctaaatctgaggtggaggagagagaagaggagagagagtaaaagcggcctgtaagcttacagcggctcagacacaaaaaccaaaaaaatctatgagacagacaagtgggccatgtattaatggtgacgagctaaccattatatgagtaggctgcaagaaggctgtaaaaaaatcttacagccaacagctgactgtattataaaacttgctctcaacaagcaaaggacagcagcgcgtcaatgcattatgaatggccagatgggccgggtccaatgagcagcatggggcagggtgcttaggtacaacagcttaatgagctgttatgagttgttggttttttttctaaagactcttttacaaaac

SEQ ID NO: 7 - protein, 158 aa, Setaria viridis
MGTRALVPAAAMAALVAVSAVMEAAAAATTYTVGAPDPRRQDHVHVLAGAARRGGGDQGGVRRLLQRQQHLRLPLRQRRRRPRRRRHPLLPLRPHGPLRQRHEDPDRRRRRRRAWPSRRRRRAVLLLRVQGRGGLFRRDRRRLLAPAARRRHRAAALV

SEQ ID NO: 8 - DNA, 128 bp, Setaria viridis
atggggacgagggctcttgttcctgcggcggcgatggcggcgcttgtggcggtgtccgccgtgatggaggcggcggcggcggcgacgacgtacacggtgggcgcgccggacccccggcgacaagatca

SEQ ID NO: 9 - protein, 74 aa, Setaria viridis
MGTRALVPAAAMAALVAVSAVMEAAAAATTYTVGAPDGLWDMQTDYAQWVKTRTFPPATRSCSRTRRSCTTWWR

SEQ ID NO: 10 - DNA, 183 bp, Setaria viridis
atggggacgagggctcttgttcctgcggcggcgatggcggcgcttgtggcggtgtccgccgtgatggaggcggcggcggcggcgacgacgtacacggtgggcgcgccggacgggctgtgggacatgcagaccgactacgcccagtgggtcaagaccaggaccttccccccggcgacaagatca

SEQ ID NO: 11 - DNA, 174 bp, Setaria viridis
ccatacccatatgcttgtttgttagacgcgtcttaatattgtgcttagacgtttggtgttgctatacattacgtacttaattgtgtacgtagttttgttttagcctgtacacttttgttatttcaataccttggggtacttataataatactgttattcgtttattcaacgtgc

SEQ ID NO: 12 - protein, 171 aa, Sorghum bicolor
MVVRVPMAMAAAVLLVVAAVVVVVEVAAAATYTVGAPAGLWDMQTDYADWVKSKTFHPGDSITFTYSPELHDVVEVIKAGYDACSSANNISAFRSGNDVVTLTAVGTRYFLCGLTGHCGNGMKIRVDVVAAKASSAPASSSASRAAAAGFAIMTTVAASLLLLQPAAVVVS

SEQ ID NO: 13 - DNA, 23 bp, Artificial Sequence
  Synthetic
ccggacgggctgtgggacatgca

SEQ ID NO: 14 - DNA, 23 bp, Artificial Sequence
  Synthetic
aagaccaggaccttccaccccgg

SEQ ID NO: 15 - DNA, 23 bp, Artificial Sequence
  Synthetic
cccatagcttgtttgttagacgc

SEQ ID NO: 16 - DNA, 23 bp, Artificial Sequence
  Synthetic
cttttgttatttcaataccttgg

SEQ ID NO: 17 - DNA, 23 bp, Artificial Sequence
  Synthetic
aggcaaattaatgagaggctggg

SEQ ID NO: 18 - DNA, 23 bp, Artificial Sequence
  Synthetic
tacacggtgggcgcgcccgccgg

SEQ ID NO: 19 - DNA, 23 bp, Artificial Sequence
  Synthetic
ctgagccgctgtaagcttacagg

SEQ ID NO: 20 - DNA, 23 bp, Artificial Sequence
  Synthetic
atgcattatgaatggccagatgg

SEQ ID NO: 21 - DNA, 157 bp, Sorghum bicolor
acacgtatatatgtgaacgagttttctggctgcgcagcagctgggtatatacgtgcaggcatatatatagatgatcccactactccagtatacttcgaccctctctagctgacccagcctctcattaatttgcctcagtagtcttacgtgtatatat

SEQ ID NO: 22 - DNA, 187 bp, Sorghum bicolor
atggttgttcgagttcctatggctatggcggcggcggtactgctcgtggtggccgccgtcgtggtggtggtggaggtggccgcggcggcgacgtacacggtgggcgcgcccgccgggctgtgggacatgcagaccgactacgccgactgggtgaagagcaagacctttcaccccggcgacagcatca